SEQUENCE LISTING (1 sequence)

SEQ ID NO: 1 - protein, 124 aa, synthetic construct
EVQLVEFGGGLVQAGGSLRLSCAASGRAFSTNPMGWFRQSPGKEREFVGAVRWSDGSTYSADSVKGRFTVFRDNAKNTVYLQMNSLKSEDTAVYYCAAGRPWSAYHSPKEYVYWGQGTLVTVSS

(19) United States
(12) Patent Application Publication
(10) Pub. No.: US 2023/0322996 A1
(43) Pub. Date: Oct. 12, 2023

(54) POLYMER-INTERACTION MOLECULE CONJUGATES AND METHODS OF USE

(71) Applicant: LIFE TECHNOLOGIES AS, Oslo (NO)

(72) Inventors: Huey Wen OOI, Oslo (NO); Pontus LUNDBERG, Strømmen (NO); Maxi-Lu BOESCHEN, Oslo (NO); Hilde ALMAASBAK, Oslo (NO); Tuva Holt HERENG, Oslo (NO); Tor Espen STAV-NORAAS, Bekkestua (NO); Ole LANDSVERK, Oslo (NO); Geir FONNUM, Fjellhamar (NO)

(21) Appl. No.: 18/053,066

(22) Filed: Nov. 7, 2022

Related U.S. Application Data
(60) Provisional application No. 63/382,416, filed on Nov. 4, 2022.
(60) Provisional application No. 63/276,739, filed on Nov. 8, 2021.

(51) Int. Cl. C08F 283/04

(52) U.S. Cl. CPC C08F 283/04

(57) ABSTRACT

The invention relates to polymer-interaction molecule (e.g., antibodies) conjugates and uses of such conjugates for delivery of interaction molecules to cells. The invention also relates uses polymer-interaction molecule (e.g., antibodies) conjugates for eliciting cellular responses (e.g., induction of cell proliferation).

### CROSS REFERENCE TO RELATED APPLICATIONS

This application claims the benefit of priority to U.S. Provisional Patent Application No. 63/276,739 filed Nov. 8, 2021 and to U.S. Provisional Patent Application No. 63/382,416, filed Nov. 4, 2022, which disclosures are hereby incorporated by reference in their entirety.

### SEQUENCE LISTING

This application contains a Sequence Listing XML file which has been submitted electronically and which material in the file is hereby incorporated by reference in its entirety. The Sequence Listing XML file was created on Apr. 26, 2023, is named TP109253USUTL1_SL26 and is 2,133 bytes in size.

### FIELD OF THE INVENTION

The subject matter set out herein relates to polymer-interaction molecule (e.g., antibodies) conjugates and uses of such conjugates for delivery of interaction molecules to cells. The subject matter set out herein also relates uses polymer-interaction molecule (e.g., antibodies) conjugates for eliciting cellular responses (e.g., induction of cell proliferation).

### BACKGROUND

Adoptive cell therapy, such as adoptive immunotherapy, holds great potential as a therapeutic modality for the treatment of a variety of diseases including cancer and chronic viral infections. One therapeutic approach involves ex vivo activation of T cells, followed by patient infusion.

Using T cells as an example, agonist antibodies to CD3 and CD28 for the activation and expansion of T cells have historically been attached to rigid materials such as polystyrene plastic and glass. A number of materials and methods have been designed to attempt to more closely replicate natural conditions under which T cells are activated. One of these involves the use of “soft beads” designed to more closely mimic cellular interactions than rigid beads (see PCT Publication WO 2013/036585). Polymers are another type of material used to which anti-CD3 antibodies and anti-CD28 antibodies have been conjugated (see PCT Publication WO 2014/048920).

However, there is a need to develop polymer-interaction molecule conjugates that allow for efficient cell signaling interactions. One advantage of such polymer-interaction molecule conjugates is enhanced commercial implementation of adoptive cell therapies. Also, polymers, especially synthetic polymers, can be selected and/or produced so that they are animal origin free and stable. Further, polymer stability can potentially be tuned or tailored through structural and chemical characteristics of the polymers.

### SUMMARY

Provided herein, in part, are compositions, and methods for preparing such compositions, in which interaction molecules (e.g., proteins, such as antibodies) are conjugated to polymers to generate polymer-interaction molecule conjugates. Also provided herein are methods involving contacting cells with polymer-interaction molecule conjugates, as well as compositions generated by such methods (e.g., mixtures composed of polymer-interaction molecule conjugate and cells; activated cells (e.g., T cell and NK cells) and cell populations; etc.).

Further provided herein are polymer-interaction molecule conjugates comprising a dendritic polymer (e.g., a G1, a G2, a G3, a G4, a G5, a G6, a G7, a G8, a G9, a G10, a G11, or a G12 dendrimer (e.g., a polyester dendrimer)). In many instances, interaction molecule component of the polymer-interaction molecule conjugates may comprise a protein that is capable of binding to the surface of a mammalian cell (e.g., a T cell, a natural killer cells, a dendritic cell, an antigen presenting cell, etc.).

Further, polymer-interaction molecule conjugates (such as the above) may comprise one or more interaction molecule selected from the group consisting of: (a) a variable heavy-heavy (VHH) chain antibody, (b) an antiCD3 antibody, (c) an antiCD4 antibody, (d) an antiCD5 antibody, (e) an antiCD56 antibody, (f) an antiCD8 antibody, (g) an antiCD25 antibody, (h) an antiCD27 antibody, (i) an antiCD28 antibody, (j) an antiCD137 antibody, (k) an anti-CD278 antibody, (1) an anti-CD134 antibody, (m) an anti-PD1 antibody, (n) an anti-CTLA-A4 antibody, (o) an anti-TIM-3 antibody, (p) and an anti-LAG-3 antibody. In particular, the interaction molecule of the polymer-interaction molecule conjugate may comprise an anti-CD3 VHH antibody and/or an anti-CD28 VHH antibody. Additionally, the interaction molecule of the polymer-interaction molecule conjugate may comprise one or more cytokine selected from the group consisting of: IL-1 beta, IL-2, IL-4, IL-5, IL-6, IL-7, IL-10, IL-12, IL-13, IL-15, IL-18, IL-21, IL-23, IL-27, IFN-gamma, and TGF-beta.

In some instances, the interaction molecule may be attached to the polymer through a covalent bond with the linker moiety, such as a linker moiety is selected from the group consisting of maleimide, haloacetamide, norbornene, succinimidyl succinate, and succinimidyl carbonate. In specific instances, one half of the linker moiety (e.g., a pharmaceutically inert linker moiety) may be attached to the polymer and the other half of the linker moiety may be attached to the interaction molecule.

Antibodies present in compositions and used in methods set out herein (e.g., anti-CD3 antibodies, anti-CD28 antibodies, anti-CD5 antibodies, anti-CD6 antibodies, anti-CD27 antibodies, anti-CD137 antibodies, etc.) may be any type of antibodies, including monoclonal antibodies, VHH antibodies, and single domain antibodies. Further, when two or more antibodies are present or used, these antibodies may be of the same type of antibody or different type of antibody (e.g., a monoclonal antibody and a VHH antibody). Additionally, when two or more antibodies are present or used, these antibodies may be conjugated to the same polymer or different polymers, where the polymers are of the same or different (e.g., dendrimers of different generations, polyoxazoline of different molecule weights, polyoxazoline and polyrotaxane, etc.). Also, when two or more antibodies are present or used, these antibodies may be present or used in the same amount or different amounts. By way of example, an anti-CD3 antibody and an anti-CD28 antibody, as well as other antibodies, may be present or used in a 1:1 ratio or ratio that varies from about 15:1 to about 1:15 (e.g., from about 3:1 to about 1:15, from about 2:1 to about 1:15, from about 3:1 to about 1:12, from about 2:1 to about 1:12, from about 1:1 to about 1:12, from about 1:1 to about 1:10, from about 1:2 to about 1:15, from about 1:3 to about 1:15, from about 1:5 to about 1:15, from about 1:4 to about 1:12, from about 1:4 to about 1:10, from about 1:5 to about 1:12, from about 1:6 to about 1:15, from about 1:6 to about 1:10, from about 1:6 to about 1:12, from about 1:8 to about 1:12, from about 1:4 to about 1:120, from about 1:4 to about 1:110, from about 1:4 to about 1:100, from about 1:10 to about 1:120, from about 1:10 to about 1:110, from about 1:10 to about 1:100, from about 1:30 to about 1:120, from about 1:30 to about 1:100, from about 1:60 to about 1:120, from about 1:60 to about 1:110, from about 1:60 to about 1:100, from about 1:80 to about 1:120, etc.).

In addition to one or more antibody (e.g., an anti-CD3 antibody, an anti-CD28 antibodies, an anti-CD5 antibody, an anti-CD6 antibody, an anti-CD27 antibody, an anti-CD137 antibody, and/or an anti-CD278 (ICOS), etc.), cells (e.g., T cells) may be also be contacted with one or more non-antibody protein (e.g., one or more cytokine, such as IL-2, IL-4, IL-6, IL-7, IL12, IL-15, IL-21, IL-23, and/or TGFβ). In many instances, one of the one or more of these antibody and non-antibody protein may be conjugated to one or more polymers.

Also provided herein are methods for activating T cells (e.g., human T cells, specific T cell subsets, etc.) in populations of T cells. These methods include those comprising contacting populations of T cells with one or more polymer-interaction molecule conjugate comprising an anti-CD3 antibody under conditions that allow for the activation of CD3 receptors on T cells in the populations. In many instances, the polymer may be a dendrimer (e.g., a G3 polyester dendrimer and/or a G5 poly(amidoamine) (PAMAM) dendrimer), a polyrotaxane, a polyoxazoline, a polystreptavidin, or a derivative of one of these polymers and may also be a copolymer or a homopolymer. Such methods may comprise contacting a population of T cells with one or more polymer-interaction molecule conjugate comprising an anti-CD28 antibody under conditions that allow for the activation of CD28 receptors on T cells in the population. Further, the anti-CD3 antibody and the anti-CD28 antibody may be conjugated to the same or different polymer molecules. Additionally, at least one of the anti-CD3 antibody or the anti-CD28 antibody may be an antibody of the type selected from the group consisting of: (a) a monoclonal antibody, (b) a single chain antibody, (c) a single domain antibody, and (d) a variable heavy-heavy domain (VHH) antibody. Additionally, T cells in the population of T cells may also be contacted with one or more cytokine. Further, at least one of these one or more cytokine (e.g., interleukin-2) may be conjugated to a polymer to form a polymer-interaction molecule conjugate.

Further, polymers present in compositions and used in methods set out herein may have a molecular weight between 0.5 kilodaltons and 150 kilodaltons (e.g., from about 0.5 kilodaltons to about 150 kilodaltons, from about 1 kilodalton to about 150 kilodaltons, from about 2 kilodaltons to about 150 kilodaltons, from about 4 kilodaltons to about 150 kilodaltons, from about 8 kilodaltons to about 150 kilodaltons, from about 15 kilodaltons to about 150 kilodaltons, from about 1 kilodalton to about 100 kilodaltons, from about 20 kilodaltons to about 80 kilodaltons, etc.).

T cells used in methods set out here may be isolated from whole blood and may further comprise T cell subset (e.g., naïve T cells, memory T cells, Th1 T cells, regulatory T cells (Tregs), CD4+ T cells, CD8+ T cells, etc.) that has been separated from other T cells prior to contacting with the one or more polymer-interaction molecule conjugate.

Further, T cells in a population of T cells may expand at least five or tenfold (e.g., from about five to about fifty, from about five to about forty, from about five to about thirty, from about five to about twenty, from about ten to about fifty, from about ten to about one hundred, from about ten to about thirty, from about fifteen to about eighty, etc. fold) after being contacted with the one or more polymer-interaction molecule conjugate. Typically, fold expansion will be measured at six, eight or ten days after the T cells contacted with the one or more polymer-interaction molecule conjugate. Thus, T cells may be assessed for fold expansion at, for example, six days after activation.

Of course, T cells may be expanded for any number of time periods, such as from about 3 days to about 30 day (e.g., from about 3 days to about 30 day, from about 6 days to about 30 day, from about 9 days to about 30 day, from about 3 days to about 21 day, from about 6 days to about 21 day, from about 10 days to about 21 day, from about 10 days to about 25 day, etc.) and fold expansion can be measured during or at the end of the expansion period.

Further, T cells generated as set out herein may be infused into a patient (e.g., a patient with cancer such as a leukemia).

Also provided herein are methods in which T cells are separated from one or more polymer-interaction molecule conjugate. In some instances, the T cells are separated from more than 50% of the one or more polymer-interaction molecule conjugate originally brought into contact with the T cells.

Further, provided herein are compositions and methods in which the ratio of the anti-CD3 antibody to the anti-CD28 antibody is from about 15:1 to about 1:15 (e.g., from about 1:1 to about 1:15, from about 1:2 to about 1:15, from about 1:3 to about 1:15, from about 1:3 to about 1:10, from about 5:1 to about 1:1, from about 15:1 to about 2:1, from about 10:1 to about 3:1, from about 1:1 to about 1:120, from about 1:30 to about 1:120, from about 1:50 to about 1:100, from about 1:70 to about 1:110, from about 1:80 to about 1:110, from about 1:90 to about 1:110, etc.).

Additionally, provided herein are methods in which T cells are also contacted with one or more protein selected from the group consisting of: an anti-CD5 antibody, an anti-CD6 antibody, an anti-CD27 antibody, an anti-CD137 antibody, an anti-CD278 (ICOS), IL-2, IL-4, IL-6, IL-7, IL12, IL-15, IL-21, IL-23, and TGFβ. Further, at least one of the one or more of these protein may be conjugated to a polymer.

Also provided herein are polymer-interaction molecule conjugates comprising one or more antibody (e.g., an anti-CD3 antibody, an anti-CD28 antibody, or both an anti-CD3 antibody and an anti-CD28 antibody, anti-CD5 antibody, an anti-CD6 antibody, an anti-CD27 antibody, an anti-CD137 antibody, etc.). In many instances, the polymer may be a polyrotaxane, a polyoxazoline, a polystreptavidin, a dendrimer (e.g., a polyester dendrimer), a polyethylene glycol, or derivatives of any of these polymers. Further, in some instances, at least one of the antibodies may be a variable heavy-heavy domain (VHH) antibody.

Polymers used in compositions set out herein may be copolymers (e.g., a random copolymer, an alternating copolymer, a gradient copolymer, a block copolymer, a graft copolymer, etc.). Further, polymers may have a disordered, linear, unbranched, branched, slightly cross-linked, highly cross-linked, star-shaped, or a molecular brush morphology. In particular instances, the polymer may be a polyoxazoline-based polymer, copolymer, or derivative thereof and may be polyoxazoline based polymers comprising at least one monomeric unit selected from any one of the monomers set out in Table 1. Polymers used in compositions set out here may also be biocompatible polymers.

Further, interaction molecules (e.g., antibodies, cytokines, etc.) may be bound to polymers through linker moieties (e.g., a small-molecule attached to the polymer and/or the interaction molecule). Interaction molecules (e.g., antibodies, cytokines, etc.) may be bound to polymers by linker moieties by covalent bonds or ionic bonds or interactions. Linker moieties that may be used include maleimide, haloacetamide, norbornene, succinimidyl succinate, and succinimidyl carbonate.

Polymer-interaction molecule conjugate provided herein may comprising a dendritic polymer, such as a dendrimer (e.g., a polyester dendrimer), a polyrotaxane or a polyoxazoline and one or more VHH antibody and, in some instances, one or more cytokine. Further, the one or more VHH antibody may be one or more of the following antibodies: an anti-CD3 antibody, an anti-CD4 antibody, an anti-CD5 antibody, an anti-CD5 antibody, an anti-CD8 antibody, an anti-CD25 antibody, an anti-CD27 antibody, an anti-CD28 antibody, an anti-CD137 antibody, or an anti-CD278 antibody. Additionally, the one or more cytokine may be one or more of the following cytokines: IL-1 beta, IL-2, IL-4, IL-5, IL-6, IL-7, IL-10, IL-12, IL-13, IL-15, IL-18, IL-21, IL-23, IL-27, IFN-gamma, or TGF-beta.

Also provided herein are methods for inducing the activation or proliferation of mammalian cells (e.g., T cells, natural killer cell, dendritic cells, antigen presenting cells, monocytes, etc.). In some instances, such methods comprise contacting the mammalian cell with a first interaction molecule capable of inducing activation or proliferation of the mammalian cell alone or in combination with a second interaction molecule. In many instances, the first interaction molecule may be a variable heavy-heavy chain (VHH) antibody capable of stimulating a cell surface receptor. In some instances, at least one of the first interaction molecule or the second interaction molecule may be conjugated to a polymer to form a polymer-interaction molecule.

Further, the first interaction molecule may be a VHH antibody with binding affinity for a CD2 or CD335 receptor. Also, the second interaction molecule may be a cytokine (e.g., IL-2, IL-12, IL-18, or IL-21).

### BRIEF DESCRIPTION OF THE DRAWINGS

FIG. 1 shows the amino acid sequence of an exemplary VHH antibody interaction molecule with binding affinity for human CD3 receptors (SEQ ID NO:1). The complementarity determining regions (CDRs) and framework regions (FRs) are shown by a combination of underlined regions and bold lettered regions.

FIG. 2 shows a reaction series in which a maleimide group is conjugated to a carboxylic acid group of a polymer (R), followed by the conjugate of a molecule containing a sulfhydryl group to the maleimide group. The hashed lined ovals represent the molecule containing the sulfhydryl group. PBS refers to phosphate buffered saline. TCEP refers to tris(2-carboxyethyl)phosphine. DMTMM refers to 4-(4,6-dimethoxy[1.3.5]triazin-2-yl)-4-methylmorpholinium.

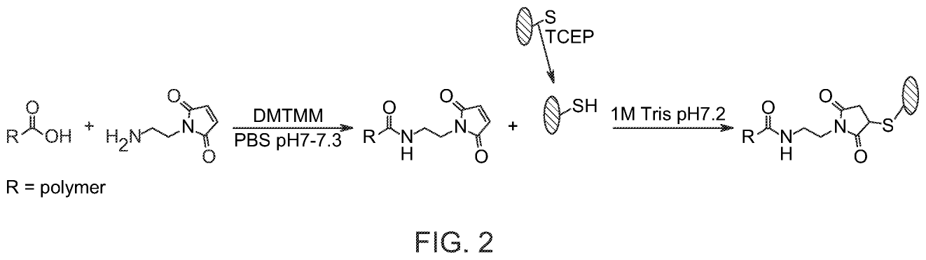

FIG. 3 shows a reaction series in which a norbornene group is conjugated to a carboxylic acid group of a polymer (R), followed by the conjugate of a molecule containing a sulfhydryl group to the norbornene group. The hashed lined ovals represent the molecule containing the sulfhydryl group. EDC refers to 1-ethyl-3-(3-dimethylaminopropyl)carbodiimide. NHS refers to N-hydroxysuccinimide. DMF refers to dimethylformamide. LAP refers to the photoinitiator lithium arylphosphinate.

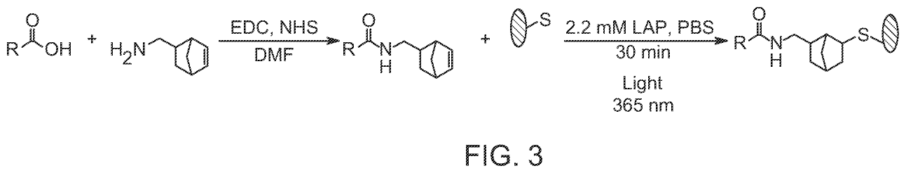

FIG. 4 shows an exemplary structure of a polymer-interaction molecule conjugate where the polymer is a polyrotaxane. The open linear line in the center of this figure labeled “Polymer” represents a polymer which has End Caps at each terminus and Chemical Rings positioned around the polymer. Also showed are a Linkers and Interaction Molecules attached to the Chemical Rings.

FIG. 5 is a schematic representation of α-cyclodextrin, which is one chemical that may be used to form Chemical Rings of polyrotaxanes.

FIG. 6 shows an exemplary schematic representation of a cell bound to a monospecific VHH antibody that is attached to a polymer (a polymer-interaction molecule conjugate). In this schematic, the VHH antibody is bound to a surface receptor of the cell (labeled “R”) by the antigen binding site of the VHH antibody. Further, the VHH antibody is attached to a linker (labeled “L”), which connects the antibody to the polymer. “Cleavage Site” refers to a location in the antibody which may be used to cleave the antibody into two parts, thereby separating the VHH antibody binding site from the region of the antibody associated with the linker.

FIG. 7 is a schematic of an exemplary cell processing workflow that contains a series of steps, starting with blood collection, which often will not be part of an automated workflow (hence the dotted line box) and ending with formulation of cells generated during the workflow. Step 3 and Step 4 may be combined into a single step in some workflows.

FIG. 8 shows T cell activation by an agonistic anti-CD3 VHH antibody in either soluble form, labeled “CD3”, or conjugated to different polymers, with activation being measured by the percentage of cells expressing CD25 receptors. POx20k-CD3 and POx100k-CD3 refer to anti-CD3 VHH antibody conjugated to, respectively, 20 kDa and 100 kDa polyoxazoline. “POx20k-CD3 (photo)” refers to a polymer-interaction molecule conjugate formed by photo ligation. Data used to generate this figure can be found in Table 5. Data is shown using, from left to right, 16.7, 8.3, 4.2, 2.1, 1.0, 0.5 μg/ml of polymer-CD3 reagents.

FIG. 9 is similar to that of FIG. 8 but with different polymers. “PRX” refers to polyrotaxane; “PEG8arm” refers to polyethylene glycol with eight “arms” connected to a hexaglycerol core where the termini of each arm are designed to contain a carboxylic acid group; “G3” and “G5” refer, respectively, to third and fifth generation dendrimers; “Polystrep” refers to a polymer of the protein streptavidin; “Strep” refers to monomeric streptavidin; and the designations “10 k”, “20 k”, “35 k” and “40 k” refer the sizes of polymers in kilodaltons. Data used to generate this figure can be found in Table 5. Data is shown using, from left to right, 16.7, 8.3, 4.2, 2.1, 1.0, 0.5 μg/ml of polymer-CD3 reagents.

FIG. 10 shows data from an experiment that was run similarly to that which yielded the data in FIGS. 8 and 9 but with lower ranges of anti-CD3-polymer conjugates. Data used to generate this figure can be found in Table 6. Data is shown using, from left to right, 16.7, 8.3, 4.2, 2.1, 1.0, 0.5, 0.3, 0.1, 0.07 μg/ml of polymer-CD3 reagents.

FIG. 11 is similar to that of FIG. 10 but with different polymers. Data is shown using, from left to right, 16.7, 8.3, 4.2, 2.1, 1.0, 0.5, 0.3, 0.1, 0.07 μg/ml of polymer-CD3 reagents.

FIG. 12 shows the percent viability of T cells eight days after being contacted with anti-CD3 and anti-CD28 VHH antibodies, where these antibodies were in soluble form, conjugated to a polymer, or a combination of thereof. The numbers at the bottom of the graph represent the following cell exposure conditions: (1) Soluble anti-CD3 and anti-CD28 VHH antibodies, (2) soluble anti-CD28 VHH antibody, (3) soluble anti-CD3 VHH antibody, (4) anti-CD3 and anti-CD28 VHH antibodies conjugated to different 10 kDa polyrotaxane polymers, (5) anti-CD3 and anti-CD28 VHH antibodies conjugated to different 20 kDa polyoxazoline polymers, (6) anti-CD3 and anti-CD28 VHH antibodies conjugated to different 100 kDa polyoxazoline polymers, (7) anti-CD3 and anti-CD28 VHH antibodies conjugated to different 20 kDa 8 arm polyethylene glycol polymers, (8) anti-CD3 and anti-CD28 VHH antibodies co-conjugated to the same 20 kDa polyoxazoline polymers, (9) anti-CD3 and anti-CD28 VHH antibodies conjugated to different fifth generation (G5) dendrimer polymers, and (10) anti-CD3 VHH antibody conjugated to 20 kDa polyoxazoline polymer and soluble anti-CD28 VHH antibody. Data used to generate this figure is set out in Table 7.

FIG. 13 shows fold expansion data generated in the same experiment used to generate the data set out in FIG. 12. Also, the numbers at the bottom of the graph represent the following cell exposure conditions as those set out in FIG. 12. Data used to generate this figure is also set out in Table 7.

FIG. 14 shows an exemplary fifth generation (G5) dendrimer with terminal carboxylic acid groups that have been partially derivatized with maleimide groups.

FIG. 15 shows CD69 receptor expression of T cells 1 day after contact with two different lots of anti-CD3 and anti-CD28 VHH antibodies conjugated to G5 dendrimers, anti-CD3 and anti-CD28 VHH antibodies to the same G5 dendrimers, and commercially available CTS DYNABEADS™ CD3/CD28. Data used to generate this figure is also set out in Table 8.

FIG. 16 is similar to FIG. 15 except that CD25 receptor expression 3 days after contact with anti-CD3 and anti-CD28 VHH antibodies is shown. Data used to generate this figure is also set out in Table 9.

FIG. 17 shows the fold expansion of T cells CD69 after 6 days after of contact with two different lots of anti-CD3 and anti-CD28 VHH antibodies conjugated to G5 dendrimers, anti-CD3 and anti-CD28 VHH antibodies to the same G5 dendrimers, and commercially available CTS DYNABEADS™ CD3/CD28. Data used to generate this figure is set out in Table 10.

FIG. 18 shows CD69 receptor expression of T cells at time periods of 4 hours, 1 day, 2 days, 3 days, and 6 days after contact with different concentrations of G5 dendrimers conjugated to receptor stimulatory anti-CD3 and anti-CD28 VHHs. The numbers at the bottom of this figure amount of dendrimer represents the nanograms/milliliter of dendrimer conjugate used. These data represented as mean fluorescent intensity (MFI). Data used to generate this figure is also set out in Table 11.

FIG. 19 is similar to FIG. 18 except that CD25 receptor expression is shown. Data used to generate this figure is also set out in Table 11.

FIG. 20 is similar to FIG. 18 except that the fold expansion of the T cells is shown. Data used to generate this figure is also set out in Table 13.

### DETAILED DESCRIPTION

### Definitions

As used herein, the term “interaction molecule” refers to a chemical entity which is to be conjugated to a polymer. Such chemical entities include proteins (e.g., antibodies, growth factors, cytokines, etc.) and non-protein pharmaceuticals. Interaction molecules may be peptides, differentiation factors, and lipids (e.g., bacterial lipids, fungal lipids, etc.).

In many instances, interaction molecules will be molecules that bind to or have an effect on a cell surface by, for example, interacting with a cell surface receptor (e.g., a receptor agonist or antagonist). Examples of cell surface receptors that interaction molecules may affect are CD3, CD5, CD278 (ICOS), CD6, CD28 and CD137 receptors. Additional and more specific examples of cell surface receptors that interaction molecules may affect are CD1 (e.g., CD1a, CD1b, CD1c, CD1d, and CD1e), CD2, CD3 (e.g., CD3d, CD3e, and CD3g), CD4, CD7, CD8 (e.g., CD8a and CD8b), CD14, CD16, CD19, CD21 (Complement Receptor 2), CD23, CD24, CD27, CD29 (integrin beta 1), CD30, CD33, CD34, CD42 (e.g., CD42a, CD42b, CD42c, and CD42d), CD44, CD45, CD51, CD63, CD79 (e.g., CD79a and CD79b), CD80, CD86, CD94 (KLRD1), CD95, CD97, CD114 (G-CSF receptor), CD115 (CSF1 receptor), CD116, CD117, CD118, CD119, CD120 (e.g., CD120a and CD120b), CD121 (e.g., CD121a and CD121b), CD122, CD123, CD124, CD125, CD126, CD127, CD128, CD130, CD131, CD132, CD134, CD135, CD138, CD140 (e.g., CD140a and CD140b), CD150, CD152, CD153, CD154, CD157, CD158 (e.g., CD158a, CD158b1, CD158b2, CD158b, CD158c, CD158d, CD158e1, CD158e2, CD158f1, CD158f2, CD158g, CD158h, CD158i, CD158j, and CD158k), CD160, CD161, CD167 (e.g., CD167a and CD167b), CD172 (e.g., CD172a, CD172b, and CD172g), CD179 (e.g., CD179a, CD179b, CD179c, and CD179d), CD181, CD182, CD183, CD191, CD194, CD200, CD202b, CD212, CD215, CD217, CD218 (e.g., CD218a and 218b), CD220, CD221, CD222, CD223, CD226, CD227, CD244, CD247 (CD3-Zeta), CD252, CD253, CD254, CD256 (APRIL), CD257 (BAFF), CD258, CD261, CD262, CD263, CD264, CD265, CD266, CD267, CD268, CD269, CD263, CD264, CD265, CD266, CD267, CD272, CD273, CD274, CD275, CD276, CD278, CD279, CD304, CD305, CD314, CD331, CD332, CD333, CD335, CD336, CD337, CD357, CD358, CD360, and CD366.

Antibodies for use in methods provided herein may be of any species, class or subtype providing that such antibodies can react with the target of interest, e.g., CD3 or CD28 receptors, as appropriate. Thus “antibodies” for use in the compositions and methods provided herein include:
(a) any of the various classes or sub-classes of immunoglobulin (e.g., IgG, IgA, IgM, IgD or IgE derived from any animal e.g., any of the animals conventionally used, e.g., sheep, rabbits, goats, mice, camelids, or egg yolk),
(b) monoclonal or polyclonal antibodies,
(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, VHHs, or other single domain antibodies), the so called “half molecule” fragments obtained by reductive cleavage of the disulfide bonds connecting the heavy chain components in the intact antibody. Fv may be defined as a fragment containing the variable region of the light chain and the variable region of the heavy chain expressed as two chains, and
(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies, e.g., single chain antibodies, CDR-grafted antibodies, etc. A single chain antibody (SCA) may be defined as a genetically engineered molecule containing the variable region of the light chain, the variable region of the heavy chain, linked by a suitable polypeptide linker as a fused single chain molecule. Also included are variable heavy-heavy domain antibodies (VHHs) that may be monovalent or bivalent.

As used herein, the term “VHH antibody” refers to antibodies that consists only of two heavy chains and, thus, lack light chains and include single domain antibodies (sdAbs), variable new antigen receptor (VNAR) single domain antibody, and antibody fragments consisting of single monomeric variable antibody domain. Antibodies of this type can be produced by cartilaginous fish and camelids (e.g., alpacas, dromedaries, camels, llamas).

VHH antibodies many be engineered to such that both heavy domains are in the same protein molecule (a single chain antibody) and contain no constant regions. Engineered VHH antibodies may be relatively small in size (e.g., 12 to 15 kDa, about 120 amino acids) in comparison to monoclonal antibodies (see, e.g., Harmsen and De Haard, “Properties, production, and applications of camelid single-domain antibody fragments,” Applied Microbiol. Biotech., 77:13-22 (2007), U.S. Pat. No. 9,040,666). Such antibodies are also referred to herein as VHH antibodies. VHH antibodies may have one or two antigen binding sites and that may be monovalent or bivalent. Bivalents refer to having binding affinity to two different epitopes.

A number of VHH antibodies are commercially available, including VHH antibodies to adeno-associated virus capsid proteins (e.g., the VHH antibodies in CAPTURESELECT™ AAVX Ligand Leakage ELISA product, Thermo Fisher Scientific, cat. no. 810352210).

As used herein the term “single domain antibody” or “sdAb” as used herein refers to a single monomeric variable antibody domain and which is capable of antigen binding (e.g., single domain antibodies that bind to a CD3 T cell surface receptor)). Single domain antibodies include some VHHs. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca, and guanaco, as described herein. Other species besides Camelidae may produce heavy chain antibodies naturally devoid of light chain. VHHs derived from other species (such as shark species) are included within the scope of this terms. Single domain antibodies may be part of a larger binding molecule (e.g., a multispecific antibody or a chimeric antigen receptor). Single domain antibodies present in compositions and used in methods set out herein include humanized single domain antibodies. General strategies to humanize single domain antibodies from Camelidae species have been described (see, e.g., Vincke et al., J. Biol. Chem., 284:3273-3284 (2009)).

In some embodiments, the single domain antibody (e.g., VHH) provided herein has a structure of FR1-CDR1-FR2-CDR2-FR3-CDR3-FR4.

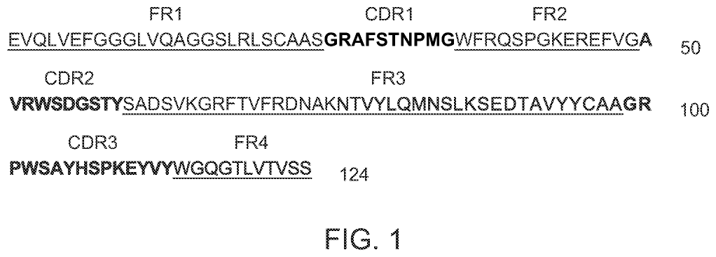

An exemplary VHH antibody interaction molecule has the amino acid sequence set out in FIG. 1.

Antibodies, as other proteins, conjugated to polymers may be designed to contain a conjugation site. In many instances, added conjugation sites will be located at the amino or carboxy terminus of the proteins. Further, conjugation sites may contain one or more cysteine residues.

The term “activation,” as used herein, refers to the state of a cell following sufficient signal induction (e.g., of a cell surface groups such as a receptor) to result in a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up- or down-regulation of expression of cell surface molecules such as receptors (e.g., CD69, CD25, CD134, CD137, HLA-DR receptors) or adhesion molecules, or up- or down-regulation of secretion of certain molecules, and performance of regulatory or cytolytic effector functions. Activation markers of NK cell include increased expression of CD69 and killer cell lectin-like receptor G1 (KLRG1). Within the context of these cells and other cells, this term infers either up- or down-regulation of a particular physico-chemical process.

The term “stimulation,” as used herein, refers to a primary response induced by signal induction. For example, in the context of receptors, such stimulation may entail the binding of a receptor with an interaction molecule and a subsequent signal transduction event. Simulation may result in cell proliferation and/or differentiation. With respect to stimulation of a T cell, such stimulation refers to the ligation of a T cell surface group that in one embodiment subsequently induces a signal transduction event. Further, the stimulation event may activate a cell and up- or down-regulate expression of cell surface molecules such as receptors or adhesion molecules, or up- or down-regulate secretion of a molecule, such as down regulation of Tumor Growth Factor beta (TGF-β). Thus, ligation of cell surface groups, even in the absence of a direct signal transduction event, may result in the reorganization of cytoskeletal structures, or in the coalescing of cell surface groups, each of which could serve to enhance, modify, or alter subsequent cell responses.

The terms “selective expansion” and “selectively expanding” as used herein in reference to T cells, refer to the ability of certain T cells to expand under condition where other T cells either will not expand or will expand at a lower rate. As a generic example, assume T cell of two different subtypes (e.g., subtype 1 and subtype 2) are present in a mixed population in respective percentages of 5% and 10% of the total T cells present. If certain conditions result in T cell subtype 1 expanding to represent 30% of the total T cells present and T cell subtype 2 representing 12% of the total T cells present, then T cell subtype 1 has been selectively expanded over T cell subtype 2, even though T cell subtype 2 is now a larger portion of the total T cell population. T cell subtype 2 is selectively expanded over general members of the mixed population of T cells in the sense that, as a total percentage of T cell, T cell subtype 2 became present with an increased “frequency”. Thus, selective expansion relates to the expansion of a particular T cell subtype over the general population of T cells and will often result in other T cell subtypes also expanding. The above example can be referred to as conditions for the selective expansion of T cell subtype 1, even though T cell subtype 2 also expands. The terms “selective expansion” and “selectively expanding” may also be used in reference to the expansion of one cell type over another cell type (e.g., NK cells over T cells).

The term “exposing” as used herein, refers to bringing into the state or condition of immediate proximity or direct contact.

The term “proliferation” as used herein, means to grow or multiply by producing new cells. The terms “proliferation” and “expansion” may be used herein interchangeably.

The term “biocompatible” as used herein refers to the properties of the material (e.g., a polymer) that it is non-toxic or has low toxicity to cells or mammals and does not induce strong alterations of the cell function, except when conjugated to one or more interaction molecules. Biocompatible materials can be derived from natural or synthetic materials that degrade in biological fluids, e.g. cell culture media and blood. In addition, biocompatible materials may be biodegradable, e.g., degraded by enzymatic activity or cleared by phagocytic cells. Degradation may occur using enzymatic means or may occur without enzymatic means. Biodegradable materials may degrade within days, weeks or few months, which may depend on the environmental conditions it is exposed to. Further, biocompatible materials may be cleared from circulation by organs such as the liver or kidneys.

The term “non-toxic,” as used herein, with respect to mammals refers to an LD₅₀ greater than or equal to 2 g/kg with a single intravenous introduction into Sprague-Dawley rats. By way of example, toxicity studies performed using a 10 kDa polyoxazoline in 0.9% (w/v) sodium chloride administered intravenously to Sprague Dawley rats, in single injection doses of 2 mg/kg produced no detectable toxic effects (Viegas, et al., Bioconjugate Journal, 22:976-986 (2011). Thus, this polyoxazoline is considered to be non-toxic.

A “subject,” as used herein, can be a vertebrate, a mammal, or a human. Mammals include, but are not limited to, farm animals, sport animals, pets, primates, mice, and rats. In one aspect, a subject is a human. A “subject” can be a “patient” (e.g., under the care of a physician) but in some cases, a subject is not a patient.

A “co-stimulatory signal,” as used herein, refers to a signal, which in combination with a primary signal, such as TCR/CD3 ligation, leads to T cell proliferation and/or activation and/or polarization. By way of example, if T cells are activated by CD3 receptor stimulation, then CD28 receptor stimulation of the same cells would be a co-stimulatory signal.

“Separation,” as used herein, includes any means of substantially purifying one component from another (e.g., by filtration, affinity, buoyant density, or magnetic attraction).

### Polymers

Polymer-interaction molecule conjugates having one or more (e.g., from about one to about five, from about two to about five, from about three to about five, from about two to about four, etc.) different interaction molecules conjugated thereto are provided herein. Interaction molecules (e.g., antibodies) may be conjugated to the polymer directly or via a linker moiety.

Polymers present in compositions and used in methods provided herein may be biocompatible polymers and can be natural or synthetic polymers that are non-toxic to a subject (e.g., a human or animal) or a biological component (e.g., cell lines, plasmid constructs, etc.). Polymer can be non-immunogenic and non-thrombotic (e.g., does not interfere with platelets and clotting factors). Thus, the polymer may be pharmaceutically inert.

Polymers can be water (aqueous solution)-soluble. In some instances, the water solubility greater than 0.1 mg/ml and in most instances solubility of polymer used in the methods set out herein will be of at least 50 mg/ml or 100 mg/ml.

The solubility of a polymer will vary with a number of factors, such as whether interaction molecules are conjugated to the polymer, the temperature, the pH, and the presence of other solutes. Further, in some instances use of polymer-interaction molecule conjugates will require solubility in solutions that target cells are present in (e.g., phosphate buffered saline, cell culture media, plasma, etc.).

Cells present in compositions and used in methods set out herein may be obtained from any number of sources, including whole blood, cord bloods, and cell culture. Further, such cells may be generated from progenitor cell lines (e.g., induced pluripotent stem cells (iPSCs)).

Polymers and polymer-interaction molecule conjugates may also have low water solubility (e.g., solubility equal to or below 0.1 mg/ml). When low water solubility polymers are employed in the conjugate, the conjugate may require the use of pharmaceutical excipients, such as oils, surfactants and/or emulsifiers.

Using phosphate buffered saline (137 mM NaCl, 2.7 mM KCl, 8 mM Na₂HPO₄, and 2 mM KH₂PO₄, pH7.4, at 1 atmosphere pressure and 25° C.) as a solute benchmark, polymers and polymer-interaction molecule conjugates provided herein may have a solubility of from about 0.05 mg/ml to about 100 mg/ml (e.g., from about 0.1 mg/ml to about 100 mg/ml, from about 0.2 mg/ml to about 100 mg/ml, from about 1 mg/ml to about 100 mg/ml, from about 5 mg/ml to about 100 mg/ml, from about 10 mg/ml to about 100 mg/ml, from about 20 mg/ml to about 100 mg/ml, from about 30 mg/ml to about 100 mg/ml, from about 40 mg/ml to about 100 mg/ml, from about 1 mg/ml to about 80 mg/ml, from about 1 mg/ml to about 60 mg/ml, from about 1 mg/ml to about 50 mg/ml, from about 1 mg/ml to about 40 mg/ml, from about 1 mg/ml to about 30 mg/ml, from about 1 mg/ml to about 20 mg/ml, from about 5 mg/ml to about 90 mg/ml, from about 5 mg/ml to about 75 mg/ml, from about 5 mg/ml to about 60 mg/ml, from about 5 mg/ml to about 40 mg/ml, from about 5 mg/ml to about 30 mg/ml, from about 10 mg/ml to about 90 mg/ml, from about 10 mg/ml to about 75 mg/ml, from about 10 mg/ml to about 60 mg/ml, from about 10 mg/ml to about 45 mg/ml, from about 15 mg/ml to about 90 mg/ml, from about 15 mg/ml to about 75 mg/ml, from about 15 mg/ml to about 55 mg/ml, from about 25 mg/ml to about 90 mg/ml, from about 25 mg/ml to about 75 mg/ml, etc.).

Polymers can, whether synthetic polymers or natural based polymers, be biodegradable or non-biodegradable. In some embodiments, polymers are biodegradable synthetic polymers (e.g., can be enzymatically degraded). In some embodiments, polymers are non-biodegradable synthetic polymers (e.g., non-enzymatically degraded).

In general, polymers are compounds prepared by the connection or polymerization of monomers, whether of the same or a different type, that in polymerized form provide the multiple and/or repeating “units” or “mer units” that make up a polymer. The terms “monomer,” “unit,” and “residue” refer to the repeating unit of the polymer. The generic term polymer thus includes the term homopolymer, usually employed to refer to polymers prepared from only one type of monomer, and the term copolymer, usually employed to refer to polymers prepared from at least two types of monomers.

It also includes all forms of copolymer, e.g., random, block, alternating, etc. It is noted that although a polymer is often referred to as being “made of” one or more specified monomers, “based on” a specified monomer or monomer type, “containing” a specified monomer content, or the like, in this context, monomer is understood to be referring to the polymerized remnant of the specified monomer and not to the un-polymerized species. In general, polymers are based on “units” that are the polymerized form of a corresponding monomer.

In some instances, polymers can include proteins such as streptavidin and polystreptavidin. Polystreptavidin is, in most instances, composed of polymerized streptavidin and normally has a high biotin binding capacity. Polystreptavidin is commercially available (Eagle Biosciences, cat. no. 10 120). When streptavidin, polystreptavidin, or similar molecules are used as polymers, the interaction molecules will typically be biotinylated with biotin or biotin derivative (e.g., N-ethyl biotin, desthiobiotin, biotin sulfone, bisnorbiotin, caproylamidobiotin, 2-iminobiotin, biocytin, N-hydroxysuccinimide-iminobiotin, etc.).

Polymers that may be present in compositions and used in methods set out herein can be homopolymers or copolymers and can be generally represented by the formula [M]ₙ, where “M” is the monomer and “n” is the degree of polymerization, i.e., the number of monomers in the polymer. When “n” is used in the context of degree of polymerization, “n” can be calculated as the ratio between the number average molecular weight (Me) and the molecular weight of the monomer. The present polymers may have a molecular weight of 5 kDa to 200,000 kDa prior to conjugation to interaction molecules. These polymers may have an average molecular mass of from about 200 daltons to about 150,000 daltons (e.g., from about 500 daltons to about 150,000 daltons, from about 1,000 daltons to about 150,000 daltons, from about 2,000 daltons to about 150,000 daltons, from about 5,000 daltons to about 150,000 daltons, from about 9,000 daltons to about 150,000 daltons, from about 10,000 daltons to about 150,000 daltons, from about 15,000 daltons to about 150,000 daltons, from about 20,000 daltons to about 150,000 daltons, from about 30,000 daltons to about 150,000 daltons, from about 2,000 daltons to about 120,000 daltons, from about 5,000 daltons to about 120,000 daltons, from about 10,000 daltons to about 120,000 daltons, from about 15,000 daltons to about 120,000 daltons, from about 20,000 daltons to about 120,000 daltons, from about 30,000 daltons to about 120,000 daltons, from about 40,000 daltons to about 120,000 daltons, from about 3,000 daltons to about 100,000 daltons, from about 8,000 daltons to about 100,000 daltons, from about 10,000 daltons to about 100,000 daltons, from about 15,000 daltons to about 100,000 daltons, from about 20,000 daltons to about 100,000 daltons, from about 25,000 daltons to about 100,000 daltons, from about 45,000 daltons to about 100,000 daltons, from about 5,000 daltons to about 80,000 daltons, from about 10,000 daltons to about 80,000 daltons, from about 15,000 daltons to about 80,000 daltons, from about 25,000 daltons to about 80,000 daltons, from about 35,000 daltons to about 80,000 daltons, from about 800 daltons to about 10,000 daltons, from about 800 daltons to about 7,000 daltons, from about 1,000 daltons to about 15,000 daltons, from about 2,000 daltons to about 15,000 daltons, etc.). For example, the polymer may have an average molecular mass of about 500, 1000, 2000, 3000, 5000, 10,000, 15,000, 20,000, 30,000, 40,000, or 50,000, or 75,000, or 100,000 daltons. The average molecular mass may be a weight average molecular mass (Mw) or a number average molecular mass (Mn). The polymer may have a dispersity or molar mass dispersity (Mw/Mn) of less than 2, less than 1.8, less than 1.6, less than 1.5, less than 1.4, less than 1.3, less than 1.2, or less than 1.1. In certain embodiments, the polymer may be monodisperse, for example, having a PDI of less than 1.2.

Copolymers can be represented by the following formula: (AB)ₙ; where n is at least 1, may be an integer greater than 1, such as 2, 3, 4, 5, 10, 15, 20, 30, 40, 50, 60, 70, 80, 90, 100, or higher, “A” represents one unit or monomer, and “B” represents a different unit or monomer. Other copolymers can be represented by the following formula: (A)ₙ(B)ₘ; where each of n and m is at least 1, and may be an integer greater than 1, such as 2, 3, 4, 5, 10, 15, 20, 30, 40, 50, 60, 70, 80, 90, 100, or higher, “A” represents one unit or monomer, and “B” represents a different unit or monomer. Still other copolymers can be represented by the following formula: [(A)ₙ(B)ₘ,]ₗ; where each of n, m and l is at least 1, and may be an integer greater than 1, such as 2, 3, 4, 5, 10, 15, 20, 30, 40, 50, 60, 70, 80, 90, 100, or higher, “A” represents one unit or monomer, and “B” represents a different unit or monomer.

Some copolymers can have As and Bs linked, or covalently bonded, in a substantially linear fashion, or in a linear manner. Some polymers can have As and Bs linked, or covalently bonded, in a substantially branched or substantially star-shaped fashion. Some polymers can have A blocks and B blocks that are randomly distributed along the polymer chain.

Each monomer, for example, A or B, independently can be an olefin, an acrylate, a lactide, a vinylpyrrolidone, an alkylene oxide, a styrene, an oxazoline, an acrylamide, a hydroxyl alkyl carboxylic acid, an amino alkyl carboxylic acid, a vinyl ether, a vinyl ester, or one or more derivative of each of these monomers.

Copolymers can be statistical copolymers, random copolymers, alternating copolymers, gradient copolymers, block copolymers, or graft copolymers.

Statistical copolymers have monomer residues arranged according to a statistical rule. A statistical copolymer in which the probability of finding a particular type of monomer residue at a particular point in the chain is independent of the types of surrounding monomer residue may be referred to as a random copolymer. Alternating copolymers possess two regularly alternating monomer residues and can be represented by a formula of [AB]ₙ, where n is 1-100 or higher. Gradient copolymers have more than two species of monomer units in a regular sequence and may be represented by a formula such as [AAAA-B-AA-B-A-BB-A-BB-AA-BBBB]ₙ, where n is 1-100 or higher. Block copolymers have long sequences of different monomer units. Polymers with two or three blocks of two distinct chemical species (e.g., A and B) can be called diblock copolymers and triblock copolymers, respectively. Polymers with three blocks, each of a different chemical species (e.g., A, B, and C) are termed triblock terpolymers. Block copolymers may be represented by the formula [AAA-BBB]ₙ, where n is 1-100 or higher. Graft copolymers contain side chains or branches whose repeat units have a different composition or configuration than the main chain. The branches are added on to a preformed main chain macromolecule.

Polymer components of polymer-interaction molecule conjugates present in compositions provided herein and used in methods provided herein can have various morphologies. For example, the polymers may have a disordered, linear, unbranched, branched, slightly cross-linked (e.g., an elastomer), highly cross-linked, star-shaped, or molecular brush morphology. In some instances, the polymers are linear polymers.

### Synthetic Polymers

In some embodiments, the polymer can be a polyvinyl pyrrolidone (PVP)-based polymer or a derivative thereof. PVP is a water-soluble polymer. PVP can be synthesized by polymerization of vinylpyrrolidone in water or isopropanol. An exemplary repeating unit for PVP-based polymers can be represented by the formula:

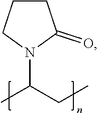

where n is degree of polymerization.

In some embodiments, the polymer can be a polyvinyl alcohol (PVA)-based polymer or a derivative thereof. PVA can be synthesized by the polymerization of vinyl acetate to polyvinyl acetate (PVAc) which is then hydrolyzed to get PVA. The extent of hydrolysis and content of acetate groups in PVA affect the crystallizability and solubility of PVA. PVA is soluble in highly polar and hydrophilic solvents, such as water, dimethyl sulfoxide (DMSO), ethylene glycol (EG), and N-methyl pyrrolidone (NMP).

The solubility of PVA in water depends on the degree of polymerization (DP), hydrolysis, and solution temperature. Any change in these three factors affects the degree and character of hydrogen bonding in the aqueous solutions, and hence the solubility of PVA. It has been reported that PVA grades with high degrees of hydrolysis have low solubility in water. The solubility, viscosity, and surface tension of PVA depend on temperature, concentration, percent hydrolysis and molecular weight of the material. An exemplary repeating unit for PVA-based

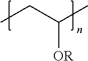

polymers can be represented by the formula: R═H or COCH₃, where n is degree of polymerization.

In some embodiments, the polymer can be a polyacrylic acid (PAA)-based polymer or a derivative thereof. PAA copolymers modified with block-copolymers of poly(ethylene oxide) (PEO) and poly(propylene oxide) (PPO) can also be employed as the components are pharmaceutically safe. Hydrophobically modified poly(acrylic acid) (HMPAA) can also be employed in the conjugates described herein. HMPAA can be prepared by modification of PAA in its acidic form by alkylamines in an aprotic solvent in the presence of N,N′-dicyclohexylcarbodiimide (DCCD). An exemplary repeating unit for PAA-based polymers can be represented by the formula:

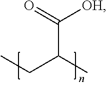

where n is degree of polymerization.

In some embodiments, the polymer can be a polyacrylamide-based polymer or a derivative thereof. Polyacrylamide, is a synthetic polymer derived from acrylamide monomer. Polyacrylamide gels result from polymerization of acrylamide with a suitable bifunctional crosslinking agent, most commonly, N,N′-methylenebisacrylamide (bisacrylamide). Gel polymerization is carried out using ammonium persulfate and the reaction rate is catalyzed by addition of N,N,N′,N′-tetramethylethylenediamine (TEMED). Polyacrylamide is stable over wide pH intervals (pH 3-11). An exemplary repeating unit for polyacrylamide-based polymers, or derivatives thereof, can be represented by the formula:

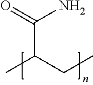

where n is degree of polymerization.

Polyacrylamide is used in wide range of cosmetic products (moisturizers, lotions, creams, self-tanning products, etc.). The Food and Drug Administration (FDA) allows polyacrylamide (with less than 0.2% acrylamide monomer) to be used as a film former in the imprinting of soft-shell gelatin capsules. The Cosmetics Ingredient Review (CIR) Expert Panel allows the use of 5 ppm acrylamide residues in cosmetic products. Recently, in addition to electrophoresis, polyacrylamides have also been used as carriers for delivery of drugs and bioactive molecules.

In some embodiments, the polymer can be an N-(2-hydroxypropyl) methacrylamide (HPMA)-based copolymer or a derivative thereof. HPMA copolymers are highly hydrophilic, non-immunogenic and non-toxic, and reside in the circulation well. HPMA copolymers contain multiple reactive groups that can be used to manipulate the properties of the polymer. Reactive functional groups commonly used for conjugation are amines, esters, imides, and phenol residues.

In some embodiments, the polymer can be divinyl ether-maleic anhydride (DIVEMA)-based polymer or a derivative thereof. DIVEMA-based polymers are water soluble and are generally 1:2 divinyl ether-maleic anhydride copolymers.

In some embodiments, the polymer can be a polyphosphate (PPE)-based polymer or a derivative thereof (e.g., polyphosphoesters or polyphosphonates). Polyphosphates have a backbone consisting of phosphorous atoms attached to either carbon or oxygen. The chemical reactivity of the phosphorous backbone enables attachment of side chains to alter the biodegradation rates and molecular weight of the polymer. PPE-based polymers are water-soluble positively charged polymers. An exemplary repeating unit for PPE-based polymers can be represented by the formula:

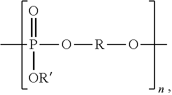

where R and R′ are each divalent organic groups, and n is degree of polymerization.

In some embodiments, the polymer can be a polyphosphazene-based polymer or a derivative thereof. Polyphosphazene-based polymers are a class of polymers with an inorganic moiety as the main chain and two active chloride groups on each repeat unit. Substitution of these chloride groups gives multifunctional polyphosphazenes with tunable physicochemical and biological properties. Some derivatives of polyphosphazenes can include water-soluble polymers such as, poly[di(carboxylatophenoxy)phosphazene] (PCPP), poly[di(methoxyethoxyethoxy) phosphazene] (MEEP), methoxypoly(ethylene glycol) and ethyl-p-aminobenzoate (mPEG/EAB-PPPs) polyphosphazenes.

In some embodiments, the polymer can be a polyglycerol-based polymer or a derivative thereof. Polyglycerol is a hyperbranched polymer that is characterized by the combination of a stable, biocompatible polyether having high end group functionality and a compact, well-defined dendrimer-like structure.

In some embodiments, the polymer can be a polyglycolic acid/or polyglycolide (PGA)-based polymer or derivatives thereof. PGA polymers are biodegradable and biocompatible aliphatic polyesters. PGA can be prepared starting from glycolic acid by ring-opening polymerization (Ikada, Y. and Tsuji, H. (2000), Macromol Rapid Commun, 21:117-132; Middleton, J C and Tipton, A J. (2000), Biomaterials. 21:2335-2346.). In some embodiments, the PGA-based polymer can be a polyglycolic acid-hyaluronan (PGA-HA) polymer, as synthesized by Patrascu et al. (2013), J Biomed Mater Res B Appl Biomater. 101:1310-1320. An exemplary repeating unit for PGA-based polymers can be represented by the formula:

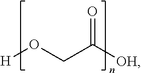

where n is degree of polymerization.

In some embodiments, the polymer can be a polylactic acid or polylactide (PLA)-based polymer or a derivative thereof. PLA-based polymers are biodegradable, bioabsorbable, thermoplastic aliphatic polyesters. Lactic acid has two optical isomers, L- and D-lactic acid. PLA can be prepared from lactide by ring-opening polymerization (Middleton and Tipton 2000). PLA-based semipermeable microcapsules are biodegradable and produce non-toxic metabolites in the body after destroyed (Chang T. (1976). J Bioeng. 1:25-32.). An exemplary repeating unit for PLA-based polymers can be represented by the formula:

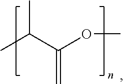

where n is degree of polymerization.

In some embodiments, the polymer can be a polycaprolactone (PCL)-based polymer or a derivative thereof. PCL-based polymers are biocompatible, bioabsorbable, and biodegradable polyesters. PCL-based polymers can be synthesized by ring-opening polymerization of ε-caprolactone using a catalyst (e.g., SnO₂) and heat (Middleton and Tipton 2000). PCL-based polymers have been used as medical implants, dental splints, targeted drug delivery, and in tissue engineering. An exemplary repeating unit for PCL-based polymers can be represented by the formula:

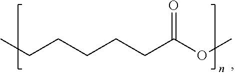

where n is degree of polymerization.

In some embodiments, the polymer can be a poly(lactic-co-glycolic acid) (PLGA)-based polymer or a derivative thereof. PLGA-based polymers are biodegradable and biocompatible copolymers. PLGA-based polymers are synthesized by ring-opening copolymerization of two different monomers of glycolic acid and lactic acid (Middleton and Tipton 2000). An exemplary repeating unit for PLGA-based polymers can be represented by the formula:

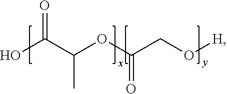

where x and y are each degree of polymerization.

In some embodiments, the polymer can be a poly(N-isopropylacrylamide) (PNIPAAm)-based polymer or a derivative thereof. PNIPAAm-based polymers are thermosensitive polymers and can be synthesized by free-radical polymerization from N-isopropylacrylamide monomers in the presence of initiators (Schild H G. (1992), Prog Polym Sci. 17:163-249). Due to unique physical and chemical properties, PNIPAAm-based polymers have been used in many applications, such as biosensors, tissue engineering, and drug delivery. An exemplary repeating unit for PNIPAAm-based polymers can be represented by the formula:

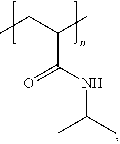

where n is degree of polymerization.

In some embodiments, the polymer is a PCL-PLA copolymer, which is biodegradable, biocompatible, and bioabsorbable. PCL-PLA copolymers can be synthesized by ring-opening polymerization.

In some embodiments, the polymer can be a polyrotaxane-based polymer or a derivative thereof.

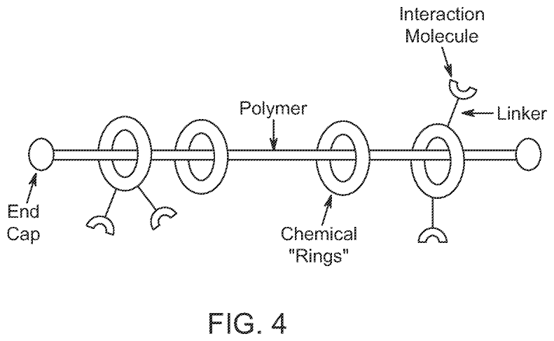

A polyrotaxane is composed of a polymer thread with chemical “rings” around the polymer. An exemplary general polyrotaxane structure is shown in FIG. 4. As can be seen from FIG. 4, the chemical rings are positioned around a polymer like beads on a string. Further, terminal groups of the polymer prevent the rings from sliding off ends of the polymer.

Any number of polymers may be used to form polyrotaxanes, including polyvinyl alcohol, polyvinylpyrrolidone, poly(meth)acrylic acid, cellulose-based resins (e.g., carboxymethylcellulose, hydroxyethylcellulose, hydroxypropylcellulose, etc.), polyacrylamide, polyethylene oxide, polyethylene glycol, polypropylene glycol, polyvinyl acetal-based resins, polyvinyl methyl ether, polyamine, polyethyleneimine, polyolefin-based resins (e.g., polyethylene, polypropylene, and copolymer resins with other olefinic monomers, polyester resins, polyvinyl chloride resins, etc.), polystyrene-based resins (e.g., polystyrene, acrylonitrile-styrene copolymer resin, etc.), acrylic (e.g., polymethyl methacrylate, copolymer of (meth)acrylate, acrylonitrile-methyl acrylate copolymer resin, etc.), polycarbonate resins, polyurethane resins, vinyl chloride-vinyl acetate copolymer resin, polyvinylbutyral resin, polyisobutylene, polytetrahydrofuran, polyaniline, acrylonitrile-butadiene-styrene copolymer (ABS resin), polyamides (e.g., nylon, etc.), polyimides, polydienes (e.g., polyisoprene, polybutadiene, etc.), polysiloxanes (e.g., polydimethylsiloxane, etc.), polysulfones, polyimines, polyacetic anhydrides, polyureas, polysulfides, polyphosphazenes, polyketones, polyphenylenes, polyhaloolefins, and derivatives thereof. In many instances, the polymer will be polyethylene glycol.

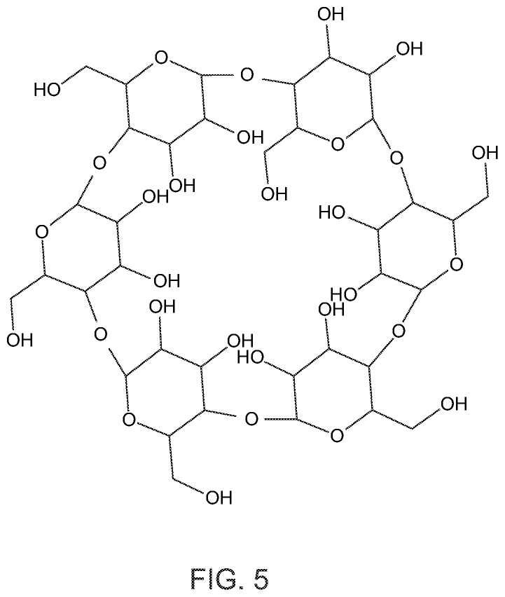

In some instances, chemical ring components of polyrotaxane may be composed of one or more of the following: cyclodextrin (e.g., α-cyclodextrin, β-cyclodextrin, γ-cyclodextrin, etc.), crown ethers, cyclophanes, calixarenes, cucurbiturils, and cyclic amides. An exemplary cyclodextrin that may be present in compositions and used in methods set out herein is shown in FIG. 5.

Further, —OH groups of chemical ring components of polyrotaxanes (e.g., of cyclodextrins) may be substituted with other groups such as —SH, —NH₂, —COOH, —SO₃H, —PO₄H. Additionally, chemical ring components may be functionalized with one or more reactive moieties (e.g., maleimide, norbornene, succinimidyl carbonate, benzotriazole carbonate, nitrophenyl carbonate, trichlorophenyl carbonate, carbonylimidazole, succinimidyl succinate, vinylsulfone, haloacetamide, and disulfide, etc.) to allow for conjugation of one or more interaction molecule.

In some instances, end caps of polyrotaxanes may be composed, of, as examples, one or more of the following: cyclodextrins, adamantane groups, trityl groups, fluorescein, pyrenes, substituted benzenes (examples of the substituents include alkyl group, alkyloxy group, hydroxy group, halogen atom, cyano group, sulfonyl group, carboxyl group, amino group, and phenyl group. One or more of the substituents may be included). In many instances, end caps of polyrotaxanes may be composed of adamantane groups and/or trityl groups.

In some embodiments, the polymer can be a poly(2-alkyl/aryl-2-oxazoline) (PAOx, POx, or POZ, also referred to as polyoxazolines) based polymer or a derivative thereof. POx may be synthesized via cationic ring-opening polymerization (CROP) of 2-oxazolines, resulting in polymers with a backbone composed of tertiary amide. An exemplary repeating unit for POx-based polymers can be represented by the formula:

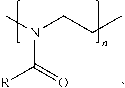

where R can be unsubstituted or substituted alkyl, unsubstituted or substituted cycloalkyl, unsubstituted or substituted heterocycloalkyl, unsubstituted or substituted aryl, or unsubstituted or substituted heteroaryl, and n is degree of polymerization.

POx functionalities can be introduced at both ends of the polymer chain by selection of the electrophilic initiator and nucleophilic terminating agent. Control of the polymer chained functionality allows incorporation of targeting units, while also enabling surface or modification. Moreover, the side chains are tunable by modification of the substituent of the 2-oxazoline monomer, granting control over the hydrophilic-hydrophobic balance and the lower critical solution temperature (LCST) of the polymer. This side-chain tunability enables the introduction of multiple functional groups along the polymer chain.

A large number of aromatic and aliphatic 2-oxazoline monomers may be used for CROP. These monomers allow one to tailor solution and aggregation properties of POx. By way of example, POx with short aliphatic side chains (C2-C4) exhibit lower critical solution temperature (LCST) in aqueous solutions. Further, longer non-polar side chains result in essentially water-insoluble polymers. In POx, the amide group connects pendant moieties to the main chain and, as a result, increasingly non-polar substituents result into an amphiphilic motive for each monomer unit. As a result, a “hydrophobic” POx can function as a non-ionic polysoap comprising a polymerized polar head group and hydrophobic tails. PMeOx and PEtOx are hydrophilic polymers and are miscible with water at all ratios and exhibit water solubility similar to PEG. Further, as the hydrophobic nature of the 2-substitution increases, the LCST decreases until water insolubility is reached. Water solubility from PMeOx to the first water insoluble PBuOx can be set out as: PMeOx>PEtOx≈PEG>PiPrOx>poly(2-cyclopropyl-2-oxazoline (PcPrOx)>PnPrOx>PBuOx (Luxenhofer et al., Macromol Rapid Commun., 33:1613-1631 (2012)). Variation of the pendant group, as well as copolymerization of hydrophilic and hydrophobic-substituted 2-oxazolines with iso- or n-propyl substituents (PiPOx, PnPOx), allows a broad adjustment of the cloud point (T_{cp}) over the entire temperature range (0 to 100° C.) as well as fine-tuning the soluble-to-insoluble transition temperature around human body temperature. Block copolymerization of hydrophilic and hydrophobic 2-oxazolines thus yields polymers of an amphiphilic contrast in the monomer unit as well as in the polymer main chain.

Amphiphilic POx can be readily obtained by the sequential block copolymerization of MeOx or EtOx with 2-oxazolines having non-polar 2-substituents such as longer 2-n-alkyl- or 2-phenyl groups, yielding defined block copolymers of low dispersity. The temperature dependent solubility of POx can be modulated over a wide range by copolymerization using EtOx, iPrOx and nPrOx with either hydrophilic or hydrophobic 2-oxazoline comonomers. Since, in POx only hydrogen-bonding acceptors but no donors are present, the cloud points of thermosensitive POx are well-defined, the soluble-insoluble transition typically occurs within <1° K and hysteresis is minimal. Amphiphilic polymers self-assemble into micelles or polymersomes in which the morphology can be selected by tuning the polymer length and composition. POx allow for highly defined polymer structure and composition enabling fine tuning of the hydrophilic-hydrophobic balance of the polymer by copolymerization and, thus, the control on micelle size and drug release properties. Most reported POx-based micellar systems feature a hybrid POx-polyester (POx-PE) diblock structure, or an ABA triblock structure synthesized by sequential addition of hydrophilic and hydrophobic 2-oxazoline monomers.

Alternatively, hydrophilic POx can be combined with hydrophobic moieties such as long alkyl chains or lipids by the initiation or termination method to yield defined non-ionic surfactants. This has been used frequently for the design of lipopolymers of defined hydrophilic-lipophilic balance for model membrane constructs.

The relatively simple monomer synthesis of POx, via a variety of routes has facilitated the preparation of a large variety of monomers that can be utilized for subsequent modifications, typically with moieties which are incompatible with CROP. For chemoselective and/or highly efficient modification, monomers bearing alkyne, alkene, thiols or aldehyde moieties may be employed. Also, to introduce pending charges along the backbone, monomers with protected amines or carboxylic acids may be employed.

An important aspect of a polymer system is the possibility to specifically tailor the polymer architecture. As numerous studies with other polymers have shown, the polymer architecture critically influences the pharmacokinetics of a polymer and thus, potentially a polymer conjugate. In POx, the living polymerization of 2-oxazolines offers a powerful and yet easy method to vary the resulting polymer architecture by various methods. A direct approach is to use initiator multiplicity to control the polymer architecture, which also allows addition of terminal functionalities, such as drug-targeting moieties at the chain ends by the termination method. Mono- and difunctional initiators yield linear symmetric or asymmetric telechelic polymers, while higher plurifunctional initiators give tri-, tetra- etc. arm star polymers, bow-tie multi-arm stars. Macroinitiators result in comb copolymers or (at high grafting densities) in molecular brushes.

The structural similarity of POx with natural polypeptides accounts for their stealth behavior and excellent biocompatibility. Along these lines, in vivo toxicity studies shown little to no adverse effects upon repeated intravenous injections (in rats) of 10 and 20 kDa PEtOx in a broad range of concentrations (500 to 2,000 mg/kg).

POx may be designed to exhibit rapid blood clearance and low uptake in organs of the reticuloendotheliary system. Further, plasma half-life may be adjusted by the use of POxs of different lengths. Along these lines, it has been shown that higher molecular weight POxs (e.g. 60 kilodaltons (kDa)) exhibit a longer plasma half-life than lower molecular weight POxs (e.g. 10 kilodaltons (kDa)) (Harris et al., European Polymer Journal 120:109241 (2019)).

POxs, as well as other polymers, that may be present in or used in methods set out herein may have an average molecular weight of from 5 kDa to about 100 kDa (e.g., from 10 kDa to about 100 kDa, from 15 kDa to about 100 kDa, from 20 kDa to about 100 kDa, from 25 kDa to about 100 kDa, from 40 kDa to about 100 kDa, from 40 kDa to about 100 kDa, from 5 kDa to about 80 kDa, from 5 kDa to about 65 kDa, from 5 kDa to about 60 kDa, from 5 kDa to about 50 kDa, from 5 kDa to about 40 kDa, from 5 kDa to about 30 kDa, from 5 kDa to about 20 kDa, from 5 kDa to about 15 kDa, from 20 kDa to about 80 kDa, from 25 kDa to about 80 kDa, from 25 kDa to about 60 kDa, from 35 kDa to about 60 kDa, etc.).

POxs, as well as other polymers, that may be present in or used in methods set out herein may have an average plasma half-life of from about 1 hour to about 30 days (from about 2 hours to about 30 days, from about 12 hours to about 30 days, from about 18 hours to about 30 days, from about 24 hours to about 30 days, from about 12 hours to about 20 days, from about 12 hours to about 15 days, from about 12 hours to about 10 days, from about 12 hours to about 7 days, from about 24 hours to about 30 days, from about 24 hours to about 20 days, from about 24 hours to about 15 days, from about 2 days to about 30 days, from about 2 days to about 20 days, from about 2 days to about 15 days, from about 5 days to about 30 days, from about 5 days to about 25 days, from about 5 days to about 20 days, from about 10 days to about 30 days, from about 15 days to about 30 days, from about 20 days to about 30 days, etc.).

Of course, the plasma half-life of a POx molecules or other polymer will vary with a number of factors. Using POxs as an example, one of these factors is the molecular weight of the polymer. Another factor is the molecule or molecules conjugated to the polymer. For example, when a protein (e.g., an antibody) is conjugated to polymer, the protein-polymer complex will have a higher molecular weight than the polymer alone. Also, the molecule or molecules may alter such characteristics as the charge (e.g., total charge, charge distribution, etc.) and hydrophobic/hydrophilic character of the polymer. Thus, when the plasma half-life of a polymer is referred to herein, it applies to the polymer alone and polymer complexes (e.g., protein-polymer complexes).

The biocompatibility of a particular material is highly complex and may vary with the interaction of the materials with a variety of biological entities such as proteins and barrier membranes. Such interactions can be hydrophobic, electrostatic or hydrogen bonding or any combination thereof. Accordingly, the ability to tailor the physicochemical characteristics of a biomaterial is highly desirable.

POx is synthesized by living polymerization, allowing for high structural and compositional definition and end-group functionalization. However, in contrast to polyethers, the water solubility of POx polymers can be specifically fine-tuned and also spans a broader range as discussed above. Combination of hydrophilic POx with (biocompatible) hydrophobic polymers yields polymer amphiphiles. Combination of POx with other hydrophobic polymers result in polymer amphiphiles that can combine advantageous properties of POx in terms of the stealth effect with already established polymer systems. Non-limiting examples of POx monomers are shown in Table 1.

TABLE 1
Exemplary POx monomers.

| POx | Structure |
|---|---|
| PMeOx | 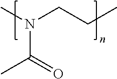 |
| PEtOx | 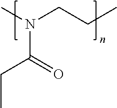 |
| PiPOx | 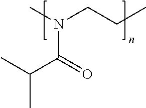 |
| P(dimethyl)Ox | 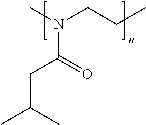 |
| P(alkyl)Ox | 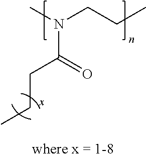 |
| P(allyl)Ox | 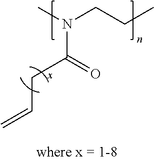 |
| P(alkyne)Ox | 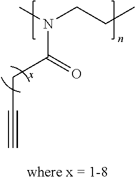 |
| PcPROx | 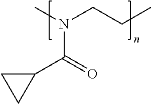 |
| PPhOx | 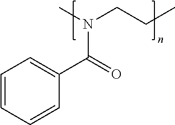 |
| PBzOx | 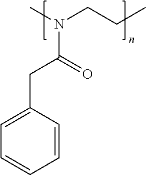 |
| P(fluoro)Ox | 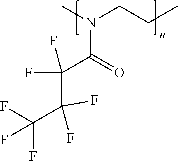 |
| P(C₂COOH)Ox | 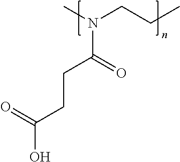 |
| P(C₃COOH)Ox | 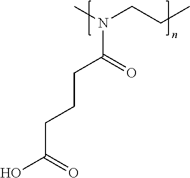 |
| P(C₂C(O)Me)Ox | 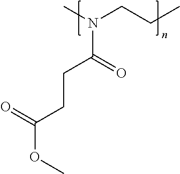 |
| P(C₃C(O)Me)Ox | 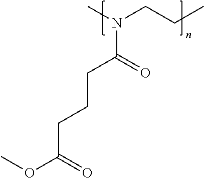 |
| P-amidatedOx | 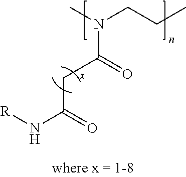 |
| P(allyl-amide)Ox | 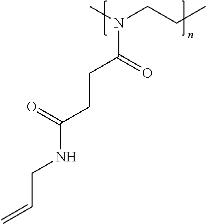 |
| P(aldehyde)Ox | 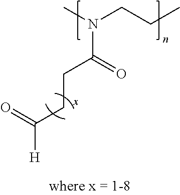 |
| P(amine)Ox | 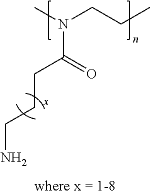 |
| P(thiol)Ox | 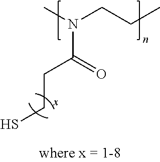 |
| P(OH)Ox | 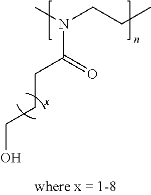 |
| P(thio-carboxyl)Ox or P(thio-acetate)Ox | 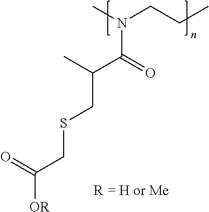 |
| P(azide)Ox | 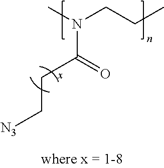 |

″n″ is degree of polymerization.

POx monomers such as those shown in Table 1 can also have protecting groups on reactive moieties (e.g., thiol, hydroxyl, amine, carboxy, allyl, etc.).

Copolymers of POx can also be used in the compositions and methods described herein. POx copolymers can have any combination of the monomers such as those shown in Table 1. POx copolymers can also have a combination of any one POx monomer such as those shown in Table 1 with a non-POx monomer unit. POx copolymers may be statistical, gradient, block, or random copolymers. When a copolymer is represented as, for example, PMeOx-PEtOx, said representation is merely an indication of a POx copolymer composition and is not reflective of a specific copolymer type. As used herein, when representing copolymers with, for example, PMeOx-PEtOx, the copolymer can be a statistical, gradient, block or random copolymer. Non-limiting examples of POx copolymers are shown in Table 2.

TABLE 2
POx Copolymers

| POx Copolymer | Monomer A | Monomer B |
|---|---|---|
| PMeOx-PEtOx | 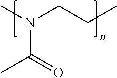 | 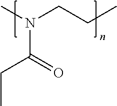 |
| PEtOx-PMeOx | 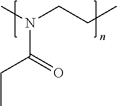 | 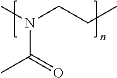 |
| PEtOx- P(C₂COOH)Ox | 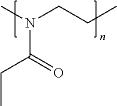 | 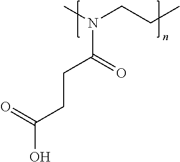 |
| PEtOx- P(C₃COOH)Ox | 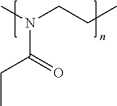 | 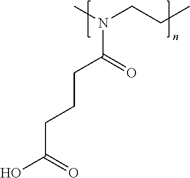 |
| PEtOx- P(C₂C(O)Me)Ox | 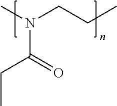 | 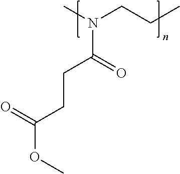 |
| PEtOx- P(C₃COOH)Ox | 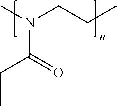 | 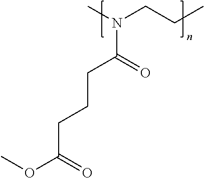 |
| PEtOx-P(allyl- amide)Ox | 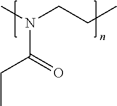 | 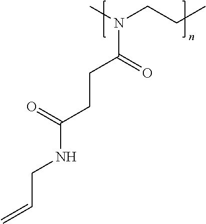 |
| PPhOx-PBzOx | 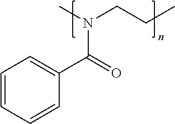 | 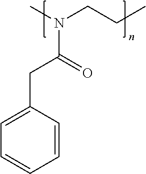 |
| PMeOx-PPhOx | 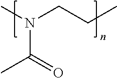 | 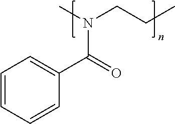 |
| PMeOx- P(fluoro)Ox | 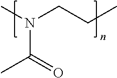 | 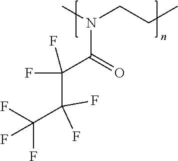 |
| P(alkyl)Ox- PPhOx | 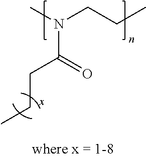 | 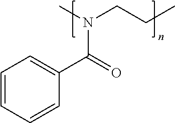 |
| P(dimethyl)Ox- PPhOx | 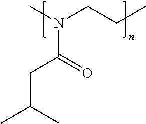 | 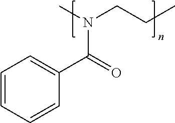 |
| PEtOx- P(dimethyl)Ox | 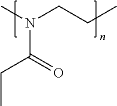 | 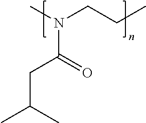 |

″n″ is degree of polymerization.

As used herein, the term “dendritic polymers” refers to highly branched polymers which can be divided into a number of sub-groups defined by their (1) structure (e.g., dendrimers, dendrons, hyperbranched polymers), (2) dispersity (e.g., monodisperse or polydisperse) or (3) internal linkages (e.g., polyethers, polyesters, polyamides) which are determined by the monomers from which they are generated and the chemistry used to generate the specific framework. Exemplary dendritic polymers include all of the polymers referred to above and dendrigrafts, linear dendritic polymers, and dendrimized polymers. M. Malkoch and S. García-Gallego, CHAPTER 1: Introduction to Dendrimers and Other Dendritic Polymers, in Dendrimer Chemistry: Synthetic Approaches Towards Complex Architectures, 2020, pp. 1-20 DOI: 10.1039/9781788012904-00001 provides a review of dendritic polymers.

Dendrimers are a category of dendritic polymers that may also be present in compositions and used in methods set out here.

Dendrimers are branched, highly ordered polymeric molecules that are typically symmetrical around and radiating out from a core. These molecules are generally characterized by having some degree of structural perfection. Along these lines, dendrimers are typically monodisperse and usually highly symmetric, spherical compounds with three dimensional structure. Thus, the term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation.

Dendrimers are normally classified by generation, which refers to the number of repeated branching addition cycles performed during its synthesis. By way of example, if a dendrimer is made three cycles of addition starting with a core, then the resulting dendrimer is considered a third generation (G3) dendrimer. Each successive generation often results in a dendrimer roughly twice the molecular weight of the previous generation. Higher generation dendrimers typically have more exposed functional groups on their surfaces for derivatization.

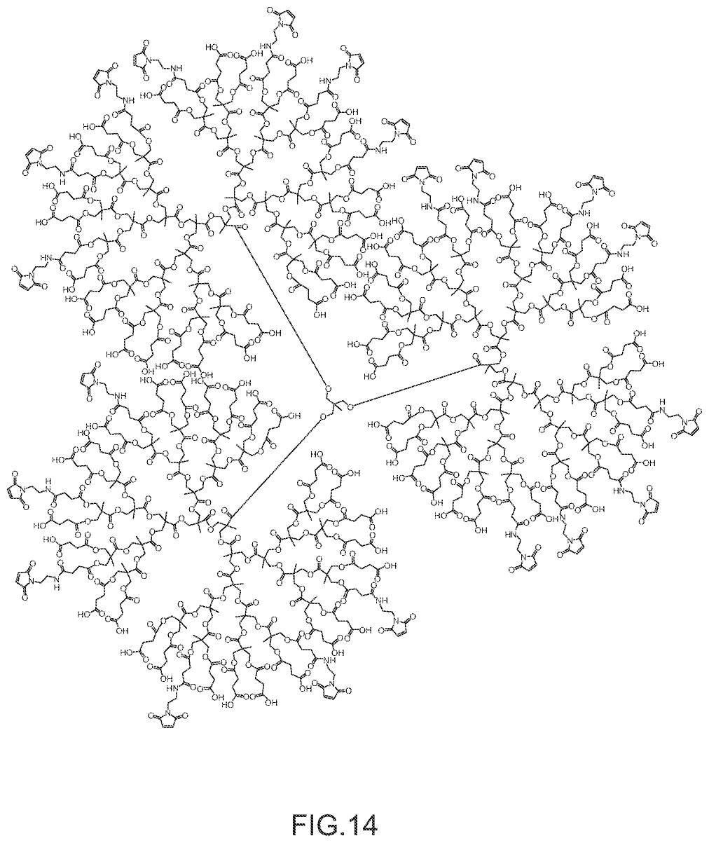

Dendrimers are normally composed a combination of dendrons. A dendron is a branched structure emanating from a single linkage to the core. Dendrimers are normally composed a combination of dendrons. A dendron is a branched structure emanating from the first generational modification of a core. Thus, FIG. 14 shows a maleimide modified, polyester based dendrimer composed of three dendrons.

Dendrimers that may be present in composition and used in methods set out herein include poly(amidoamine) (PAMAM) dendrimers, poly(propylene imine) (PPI) dendrimers, triazine dendrimers, citric acid dendrimers, polyester dendrimers, polyether dendrimers, phosphorous dendrimers, carbosilane dendrimers, and carbosiloxane dendrimers.

A number of types of dendrimers, structures of various dendrimers, and uses of dendrimers are set out in Vogtle (editor), “Dendrimers II: Architecture, Nanostructure and Supramolecular Chemistry”, 210 TOPICS IN CURRENT CHEMISTRY, Springer-Verlag 2000.

Dendritic polymers and dendrimers that may be present in composition and used in methods set out herein include G1, G2, G3, G4, G5, G6, G8, G9, G10, G11 and G12 dendrimers (e.g., polyester dendrimers) and combinations thereof (e.g., a combination of G3 and G5, G3 and G4, G5 and G7, G5 and G6, etc.). By way of example, T cells may be activated using a G3 dendrimer-anti-CD3 VHH antibody polymer-interaction molecule and a G5 dendrimer-anti-CD28 VHH antibody polymer-interaction.

A number of processes are known for producing dendrimers (see, e.g., U.S. Pat. No. 8,734,870) and dendritic polymers. Further, dendrimers of different types and derivatized with different functional groups are commercially available. Further, a number of methods are also known for the derivatization and conjugation of biological molecules to dendrimers (see, e.g., US Patent Publication 2022/0288216A1).

Exemplary cores that may be used in polymer production include disulfide and trimethylol propane cores. Dendrimer cores may be formed by reacting a diamine (e.g., ethylenediamine) with methyl acrylate. Exemplary reagents that may be used for generational addition cycles include 2,2-bis(hydroxymethyl)propionic acid. Exemplary terminal groups that may be used for derivatization include NH₂/NH₃, carboxylic acid, azide (suitable for click chemistry reaction), and hydroxyl groups. Exemplary terminal groups that may be used for conjugation include the same groups set out above for derivatization but also include maleimide, haloacetamide, norbornene, succinimidyl succinate, and succinimidyl carbonate groups.

Dendrimers present in compositions and used in methods set out herein may be polyester based.

Commercial suppliers of dendrimers include Polymer Factory Sweden AB, Stockholm, Sweden; Alfa Chemistry, Ronkonkoma, NY; and Glenn Research, Sterling, VA. A number of polyester dendrimers based on 2,2-bis(methylol)propionic acid (bis-MPA) dendrimers are available, for example, from Polymer Factory Sweden AB.

The properties of dendrimers are partially determined by their functional surface groups. Also, unlike some polymers, the water-solubility of dendrimers can be increased by functionalizing their outer shell with charged and/or hydrophilic groups.

Any number of different types of interaction molecules can be conjugated to dendrimers. These molecules include conjugating of detectable agents (e.g., dye molecules), affinity ligands (e.g., antibodies, such as variable-heavy-heavy antibodies), targeting molecules, radioligands, imaging agents, and pharmaceutically active compounds.

An exemplary G5 dendrimer molecule is shown in FIG. 14. This dendrimer molecule is a polyester dendrimer that contains terminal carboxylic acid groups derivatized with maleimide groups. As shown in FIG. 14, the degree of derivatization of dendrimers set out herein will generally not be 100% but will generally be between 20% and 80% (from about 20% to about 75%, from about 20% to about 65%, from about 20% to about 60%, from about 20% to about 50%, from about 30% to about 80%, from about 30% to about 70%, from about 30% to about 60%, from about 35% to about 75%, from about 35% to about 65%, etc.).

The degree of conjugation of dendrimers (as well as other polymers, such as dendritic polymers) set out herein will also generally not be 100%. The degree of conjugation will generally be between 20% and 90% (from about 20% to about 85%, from about 20% to about 75%, from about 20% to about 70%, from about 20% to about 60%, from about 30% to about 90%, from about 30% to about 80%, from about 30% to about 80%, from about 35% to about 85%, from about 40% to about 75%, etc.). Degree of conjugation is determined by the number of interaction molecule conjugatable groups present on the polymer. For example, if a G5 dendrimer has a degree of derivatization of 50% with maleimide groups and 50% of those groups are conjugated to, for example, an anti-CD3 VHH antibody, then the degree of conjugation would be 50%.

Other exemplary synthetic polymers that may be present in compositions and used in methods provided herein include, but are not limited to, polygalacturonic acid-based polymers; hydroxalkyl(meth)acrylate and copolymers thereof, such as poly(N-phenylpyrrolidone), poly(L-glutamic acid), poly(hydroxyethyl-L-glutamine), poly(α-malic acid), poly-L-lysine, polyethyleneimine and polyalkyl(meth)acrylate; diamido-diarnine polymer, SMANCS (styrene-co-maleic acid/anhydride polymer) or derivatives thereof.

### Natural-Based Polymers

In some embodiments, polymers contained in compositions and used in methods provided herein can be derived from natural polymers, for example, polysaccharides such as chitin, chitosan, and alginate, and proteins such as collagen and gelatin.

In some embodiments, the polymers can be derived from chitin. Chitin exists in animal skeletal systems, the lens of the eye, tendons; the outer layer of arthropods and insects and arachnids and crustaceans body (crab, shrimp, and lobster); and the internal parts of body in some animals, such as mollusks and plants, as well as in the cell wall of fungus (Malafaya et al. (2007), Adv Drug Deliv Rev. 59:207-233; Ravi Kumar M N V (2000), React Funct Polym. 46:1-27.). Chitin is a linear polymer composed of repeating β-(1,4)-N-acetylglucosamine units.

In some embodiments, the polymers can be derived from chitosan. Chitosan is a linear polysaccharide composed of randomly distributed β-(1→4)-linked D-glucosamine (deacetylated unit) and N-acetyl-D-glucosamine (acetylated unit). Chitosan is a biocompatible polymer, non-toxic, and biodegradable.

Chitin and chitosan are difficult to dissolve in water and at neutral pH. Water soluble derivatives of chitin and chitosan have been synthesized by various researchers by chemical modification (see, for example, Masatoshi et al. Carbohydr. Polym., 36:49-59 (1998); and TienAn et al. Carbohydr. Polym., 75:489-497 2009)). These chemical modifications result in the formation of hydrophilic chitin or chitosan which have more affinity to water or organic solvents or example, carboxymethylation of chitosan results in formation of N-carboxymethylchitosan (N-CMC) which is soluble in a wide range of pH.

In some embodiments, the polymers can be derived from alginate, which is a linear and homogeneous polysaccharide. Alginate can be prepared by dark and brown algae (George et al., J Control Release. 114:1-14 (2006); Shanmugam et al., Natl. Prod. Radiance. 4:478-481 (2005)).

In some embodiments, the polymers can be derived from collagen, a protein found in the extracellular matrix of animals. Collagen is composed of three polypeptide chains and can be extracted from skin, tendons, cartilage, and bone of animals. Collagen is biodegradable, biocompatible, and can easily be destroyed by enzymes.

In some embodiments, the polymers can be derived from gelatin, a solid substance that is translucent and colorless obtained from the hydrolysis of collagen (Malafaya et al. (2007), Shanmugam et al. (2005)). Gelatin forms colloids and gel in water.

In some embodiments, the polymers can be derived from xanthan. The primary structure of xanthan has repeating pentasaccharide units of two D-glucopyranosyl units, two D-mannopyranosyl units and one D-glucopyranosyluronic unit. Xanthan is a free-flowing powder soluble in both hot and cold water that gives viscous solutions at low concentrations.

In some embodiments, the polymers can be derived from pectin. Pectin is a mixture of polysaccharides. Pectins are mainly obtained from citrus peel or apple pomades, both of which are by-products of juice manufacturing process. Pectin is mainly composed of D-galacturonic acid (GalA) units joined in chains by means of α-(1-4) glycosidic linkage. These uronic acids have carboxyl groups, some of which are naturally present as methyl esters and others are commercially treated with ammonia to produce carboxamide groups. Pectins are soluble in pure water. Monovalent cation (alkali metal) salts of pectinic and pectic acids are soluble in water; di- and tri-valent cations salts are weakly soluble or insoluble.

In some embodiments, the polymers can be derived from dextran. Dextran can be produced by fermentation of media containing sucrose by Leuconostoc mesenteroides. B512F. Dextran is an α-D-1,6-glucose-linked glucan with side chains 1-3 linked to the backbone units of the dextran biopolymer. Fractions of dextran are readily soluble in water to form clear, stable solutions. The solubility of dextran is not affected by pH. They are also soluble in other solvents like methyl sulfide, formamide, ethylene glycol, and glycerol. Dextran fractions are insoluble in alcohols like methanol, ethanol and isopropanol, and also most ketones, such as acetone and 2-propanone. Dextran derivatives include dextran crosslinked with methacrylate (MA) and hydroxyethylmethacrylate (HEMA).

In some embodiments, the polymers can be derived from carrageenan. The main sources for carrageenan are the Chondrus crispus, Eucheuma cottonii and Eucheuma spinosum species. Carrageenan has repeating galactose units and 3,6-anhydrogalactose (3,6-AG), sulfated and non-sulfated, joined by alternating α-(1-)- and β-(1-4)-glycosidic linkages. There are three main types of carrageenan, called iota, kappa, and lambda carrageenan.

In some embodiments, the polymers can be derived from guar gum. Guar gum is derived from endosperm of the guar plant (Cyamopsis tetragonoloba). Guar gum is a polysaccharide composed of the sugars, galactose and mannose. Guar gum's backbone is a linear chain of β-1,4-linked mannose residues to which galactose residues are 1,6-linked at every second mannose, forming short side-branches.

In some embodiments, the polymers can be derived from cellulose ethers. Cellulose ethers are water soluble. Exemplary cellulose ether include, but are not limited to, hydroxypropylmethyl cellulose (HPMC), hydroxypropyl cellulose (HPC), hydroxyethyl cellulose (HEC), and sodium carboxy methyl cellulose (Na-CMC).

In some embodiments, the polymers can be derived from hyaluronic acid (HA), a natural polyanionic polysaccharide distributed widely in the extracellular matrix and the joint liquid of mammalians. It is a non-toxic, biocompatible mucoadhesive polysaccharide having negative charge and is biodegradable. HA is composed of two sugar units—glucuronic acid and N-acetylglucosamine which is polymerized into large macromolecules of over 30,000 repeating units.

In some embodiments, the polymers can be derived from albumin. Albumin is acidic, stable (e.g., in pH range of 4-9), thermostable (even when heated at 60° C. for up to 10 hours), biodegradable, and lacks toxicity and immunogenicity.

In some embodiments, the polymers can be derived from starch. Starch is mainly composed of two homopolymers of D-glucose: amylose, a mostly linear D-(1,4′)-glucan, and branched amylopectin, having the same backbone structure as amylose but with many α-1,6′-linked branch points. Starch has many hydroxyl functional groups in its structure and so it is hydrophilic in nature. Starch-derived polymers include starch copolymers with PCL and PLA, and starch-g-PVA. Starch-based biodegradable polymers (SBBP) have been previously synthesized (see, for example, Marques et al., Biomaterials, 23:1471-1478 (2002); Mendes et al., Biomaterials, 22, 2057-2064 (2001); Azevedo et al. Biomacromolecules, 4:1703-1712 (2003); Defaye and Wong, Carbohydr. Res., 150:221-231 (1986); Reddy et al., Drugs Today, 35:537-580 (1999)) and exhibit biocompatibility, its degradation products are non-toxic and have good mechanical properties.

Other exemplary natural-based polymers include, but are not limited to, polysaccharides, such as dextrin, dextran, chitosan derivatives, such as N-succinyl chitosan, carboxymethyl chitin, carboxymethyl pullulan, bioalgins which are polysaccharides consisting of a partially acetylated variable block copolymer of D-mannuronic and L-guluronic acid residues; Poly(amino acid(s)), such as poly(N-(2-hydroxyethyl)-L-glutamine) (PHEG), β-poly(2-hydroxyethyl aspartamide) (PHEA), poly(α-L-glutamic acid) (PGA), poly(aspartic acid), polylysine (poly(L-lysine)); or polyesters, such as α- or β-malic acid.

### Linkers

In some embodiments, interaction molecules (e.g., antibodies) are covalently linked to the polymer via a linker moiety.

The linker may be any group which links the polymer and the interaction molecule(s) and which does not adversely affect desired properties of the polymer-interaction molecule conjugate. Such linkers may include linear or branched, saturated or unsaturated, C1-15 alkyl, optionally substituted by carbonyl, amide, hydroxyl or halogen. Linkers may also be a peptide, such as a peptide of 1 to 10 amino acids in length in which the amino acids may be further substituted with amino, thio, carboxyl, carboxamide or imidazole groups. Some peptide linkers may be degraded by lysosomal enzymes.

Linkers may be attached to the polymer and the interaction molecule by conventional synthetic methods well known to the skilled person. The following bonds are example of those that may provide a suitable means for attaching the interaction molecule to the polymer: an amide bond, an ester bond, a hydrazide bond, a urethane (carbamate) bond, a carbonate bond, an imine (Schiff base) bond, a thioether bond, an azo bond or a carbon-carbon bond. In some embodiments, the interaction molecule may be attached directly to the polymer itself (e.g., the linker is a covalent bond).

In some embodiments, chemoselective ligation is employed to link the interaction molecule and polymer. In some embodiments, biorthogonal chemistry is employed to link the interaction molecule and polymer.

When employing chemoselective ligation, the polymer is functionalized with the linker so as to provide a reactive group whereby the interaction molecule can attach to. In some embodiments, the interaction molecule reacts with the linker moiety via an amino acid residue, for example a cysteine, a tyrosine, a tryptophan, or an arginine residue. In some embodiments, the interaction molecule reacts with the linker moiety via an amino acid derivative for example a disulfide bond or an N-terminus of an amino acid residue.

In some embodiments, the polymer is functionalized with a maleimide moiety. In some embodiments, the polymer is functionalized with a norbornene moiety. In some embodiments, the maleimide-functionalized polymer binds to the interaction molecule via a cysteine residue. In some embodiments, the maleimide-functionalized polymer binds to a cysteine residue that derives from a reduced disulfide bond. In some embodiments, the norbornene-functionalized polymer binds to the interaction molecule via a cysteine residue. In some embodiments, the norbornene-functionalized polymer binds to a cysteine residue that derives from a reduced disulfide bond. Non-limiting exemplary linkers/functionalized polymers are shown in the schemes below.

In some embodiments, the linker is a moiety that can react with cysteine groups in the interaction molecule, for example, with a maleimide, allyl, norbornene, etc. moieties (see Lowe, A. B., Polym. Chem., 2014, 5, 4820-4870 and Hoyle, C. and Bowman, C. (2010), Thiol-Ene Click Chemistry. Angew. Chem. Int. Ed., 49:1540-1573, each of the disclosures incorporated herein by reference). The scheme below exemplifies some of these reactive moieties and the resulting linkage between the polymer and the interaction molecule (Scheme 1).

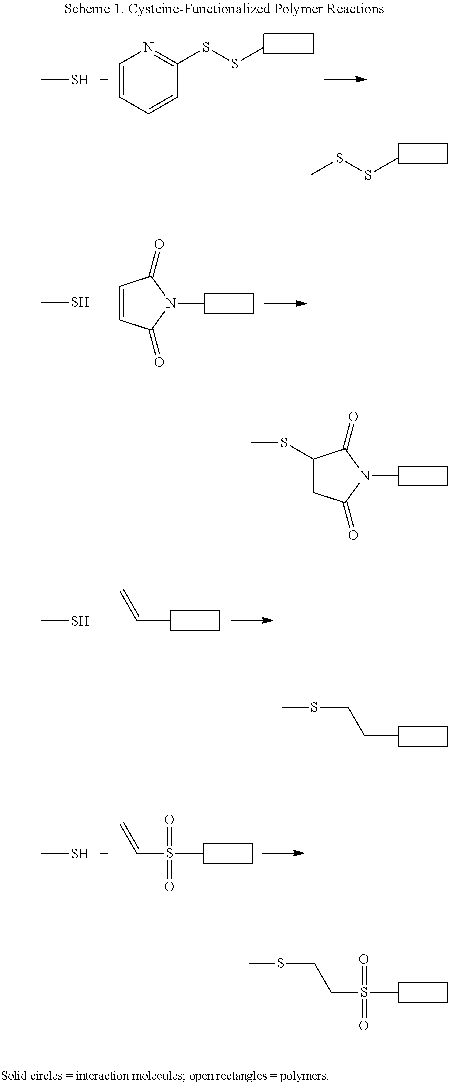

In some embodiments, a POx polymer is functionalized with linker moieties that react with cysteine groups of the interaction molecule. In some embodiments, a POx polymer is functionalized with a maleimide linker moiety:

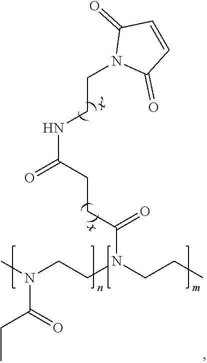

where m and n are each degree of polymerization, and x and x′ are each 1-8.

It is also envisioned that in some embodiments, not all of the functionalizable units of the polymer are functionalized with a linker moiety. For example, in some embodiments, a POx polymer is not 100% functionalized with a maleimide linker moiety:

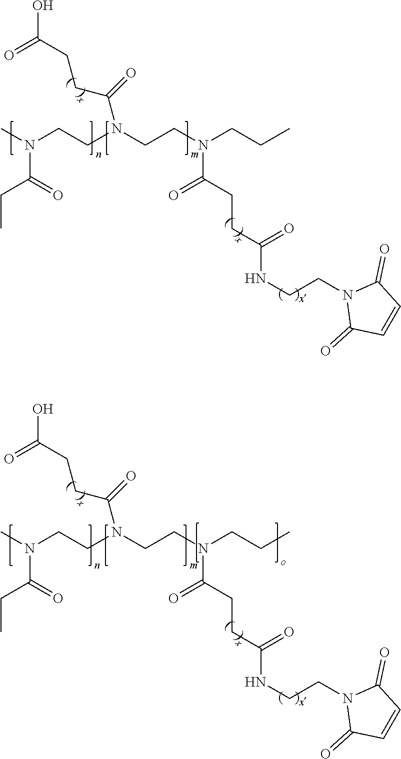

where m, n, and o are each degree of polymerization, and x and x′ are each independently 1-8.

In some embodiments, the linker is a moiety that can react with disulfide bonds in the interaction molecule. The scheme below exemplifies some of these reactive moieties and the resulting linkage between the polymer and the interaction molecule (Scheme 2).

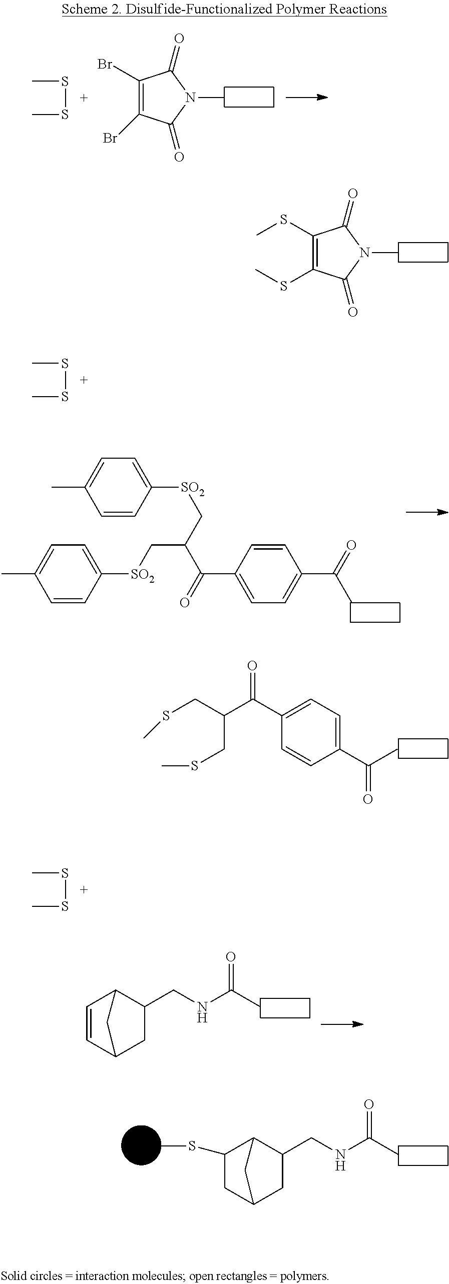

In some embodiments, a POx polymer is functionalized with a norbornene linker moiety:

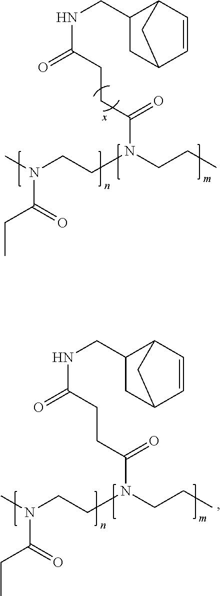

where m and n are each degree of polymerization and x is 1-8.

In some embodiments, the linker is a moiety that can react with a tyrosine residue in the interaction molecule. The scheme below exemplifies some of these reactive moieties and the resulting linkage between the polymer and the interaction molecule (Scheme 3).

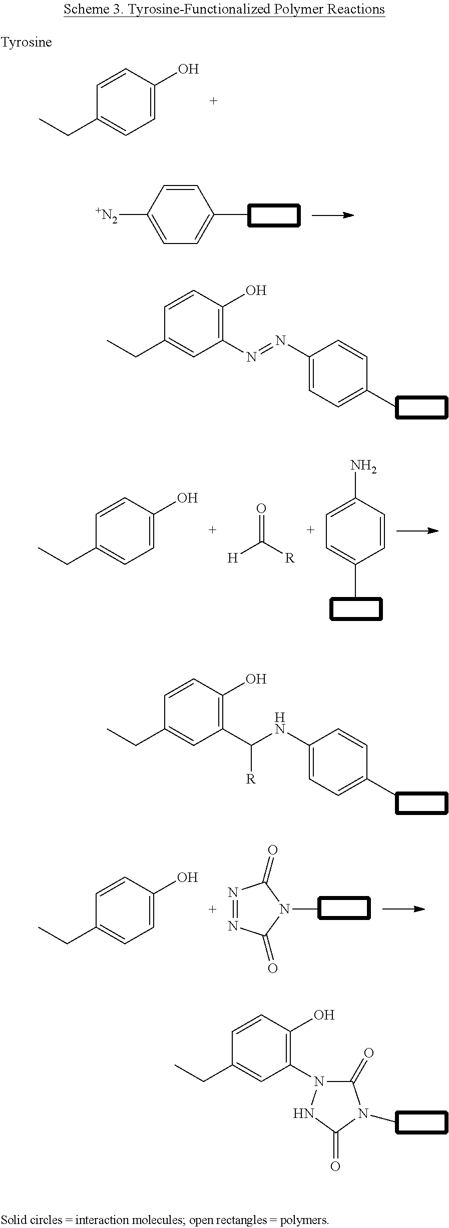

In some embodiments, the linker is a moiety that can react with a tryptophan or arginine residue in the interaction molecule. The scheme below exemplifies some of these reactive moieties and the resulting linkage between the polymer and the interaction molecule (Scheme 4).

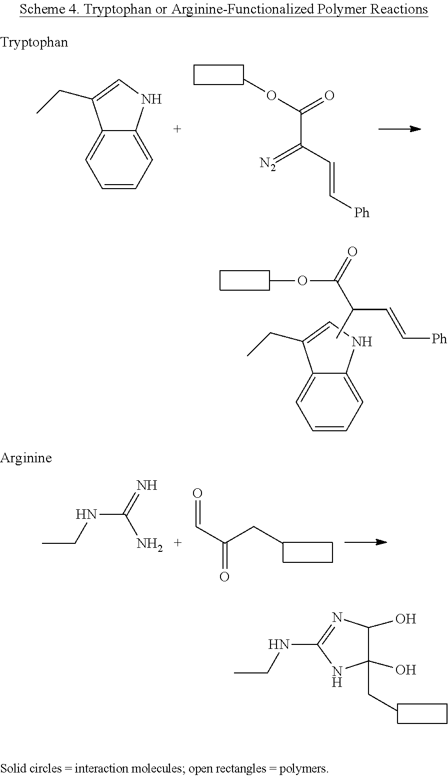

In some embodiments, the linker is a moiety that can react with an N-terminus of an amino acid in the interaction molecule. The scheme below exemplifies some of these reactive moieties and the resulting linkage between the polymer and the interaction molecule (Scheme 5).

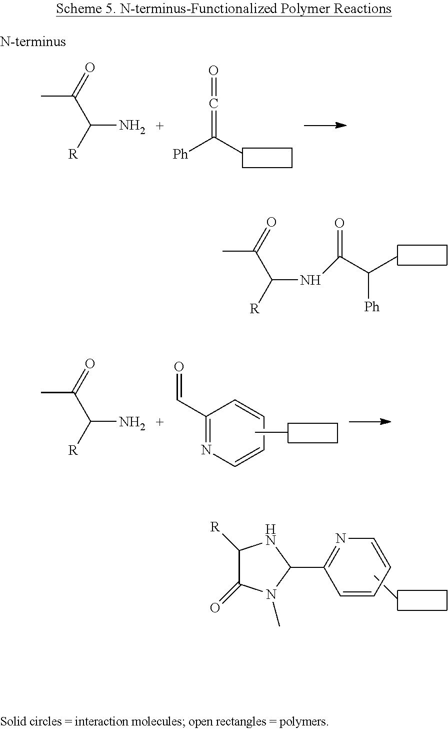

When employing bio-orthogonal chemistry, both the polymer and interaction molecule are functionalized with reactive moieties, so each moiety reacts with each other. In some embodiments, the polymer and interaction molecule are functionalized so they conjugate via an alkyne-azido reaction, a Diels-Alder reaction, a photo-click reaction, a Staudinger reaction, a Sonogashira reaction, a Suzuki-Miyaura reaction, a Trapped-Knoevenagel ligation, a Hydrazino-Pictet-Spengler ligation, or cross-methastasize. Non-limiting exemplary functionalized polymers and antibodies are shown in the scheme below (Scheme 6).

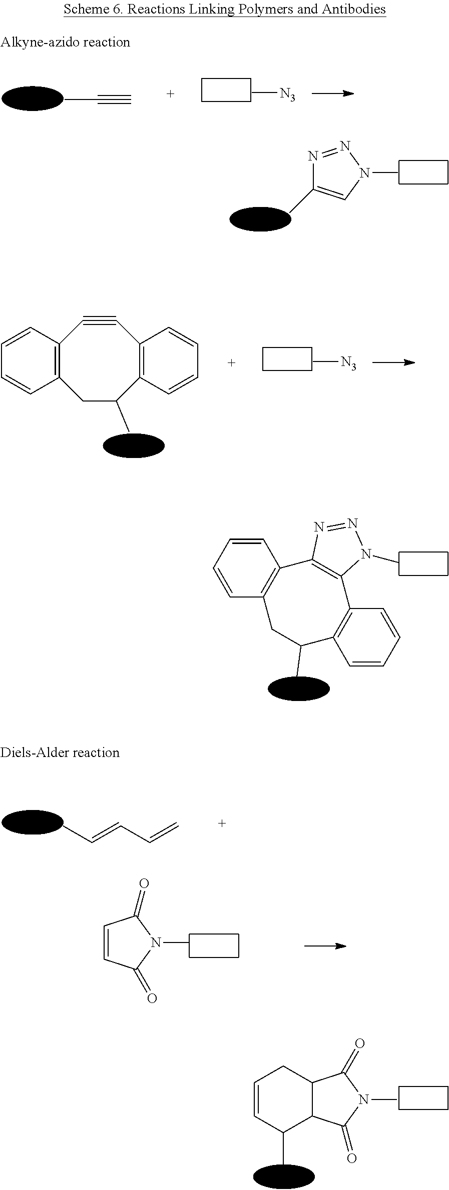

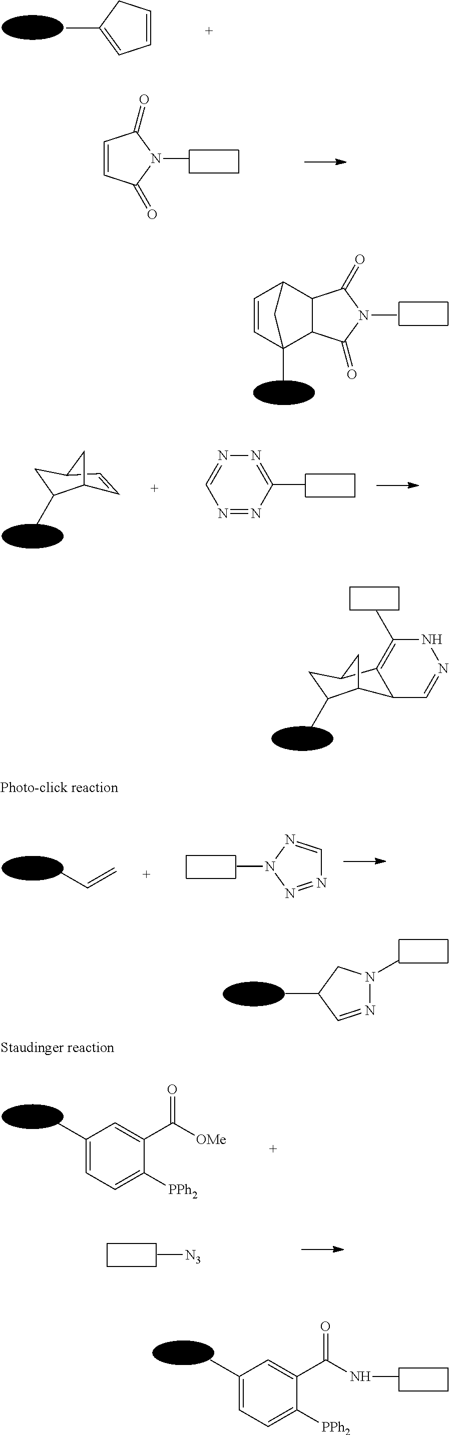

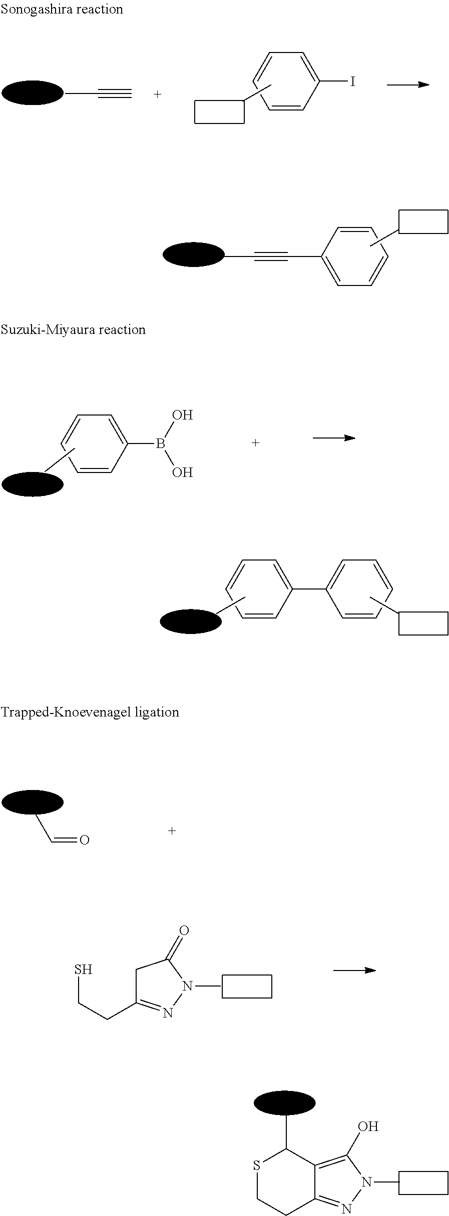

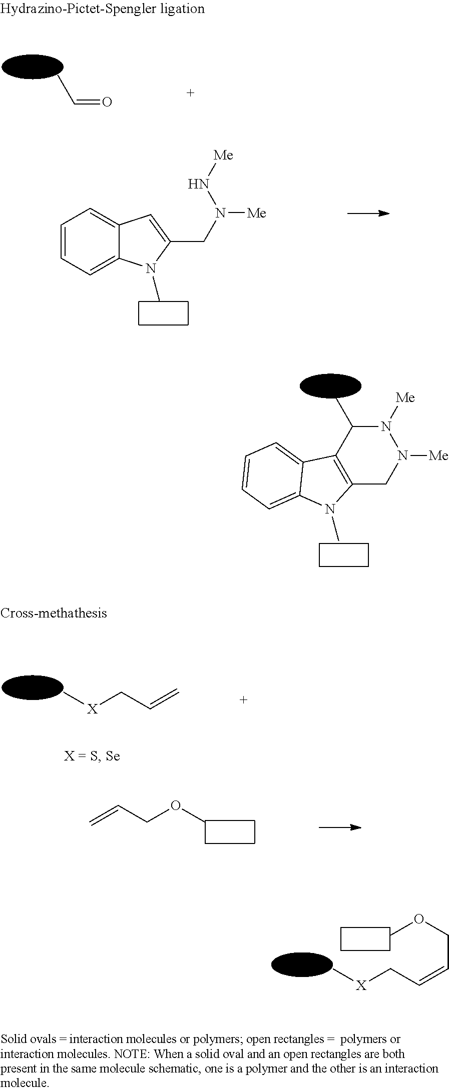

### Conjugates

As indicated above, conjugates as described herein can have an array of polymers conjugated to the interaction molecule or can have an array of polymers functionalized with an array of linkers that bind to the interaction molecule or can have an array of polymers functionalized with an array of linkers that bind to an interaction molecule that is also functionalized with an array of linkers.

In some embodiments, the conjugates comprise a polymer directly linked to the interaction molecule.

In some embodiments, and as shown below, a POx-carboxylic acid-POx-Et copolymer binds directly to cysteine residues of the interaction molecule (represented by the solid oval):

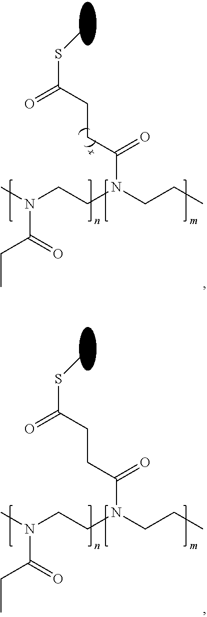

where m and n are each degree of polymerization and x is 1-8.

In some embodiments, and as shown below, a POx-alkene-POx-Et copolymer binds directly to cysteine residues of the interaction molecule (represented by the solid oval):

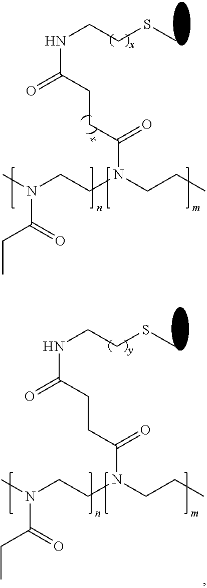

where m and n are each degree of polymerization, x is independently 1-8, and y is 1-4.

In some embodiments, the conjugates comprise a polymer functionalized with a linker, the linker moiety attached to the interaction molecule. In some embodiments, and as shown below, a POx-carboxylic acid-POx-Et copolymer is functionalized with a maleimide linker, the maleimide moiety linking the polymer to the interaction molecule (represented by the solid oval) via cysteine residues:

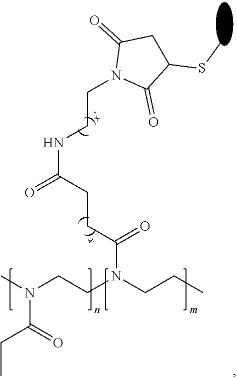

where n and m are each degree of polymerization and x and x′ are each 1-8.

In some embodiments, and as shown below, a POx-carboxylic acid-POx-Et copolymer is functionalized with a norbornene linker, the norbornene moiety linking the polymer to the interaction molecule (represented by the solid oval) via cysteine residues:

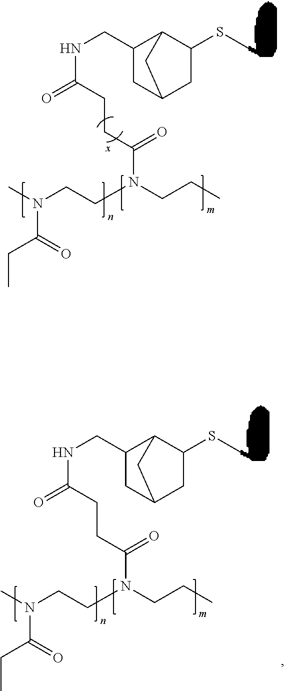

where m and n are each degree of polymerization and x is 1-8.

In some embodiments, the conjugates described herein contain a PVA-based polymer functionalized with a maleimide or norbornene linker that binds to the cysteine residues of the interaction molecule. In some embodiments, the conjugates described herein contain a PAA-based polymer functionalized with a maleimide or norbornene linker that binds to the cysteine residues of the interaction molecule. In some embodiments, the conjugates described herein contain PAA-PEO or PAA-PPO copolymers functionalized with a maleimide or norbornene linker that binds to the cysteine residues of the interaction molecule. In some embodiments, the conjugates described herein contain a HMPAA-based polymer functionalized with a maleimide or norbornene linker that binds to the cysteine residues of the interaction molecule. In some embodiments, the conjugates described herein contain a PPE-based polymer, such as polyphosphoesters or polyphosphonates functionalized with a maleimide or norbornene linker that binds to the cysteine residues of the interaction molecule. In some embodiments, the conjugates described herein contain a PLGA-based polymer functionalized with a maleimide or norbornene linker that binds to the cysteine residues of the interaction molecule.

In some embodiments, the conjugates described herein contain a POx-based polymer functionalized with an alkyne moiety and an interaction molecule functionalized with an azide moiety so the polymer and interaction molecule link via an alkyne-azido cycloaddition or via a strain-promoted alkyne-azido cycloaddition. In some embodiments, the conjugates described herein contain a POx-based polymer functionalized with an azide moiety and an interaction molecule functionalized with an alkyne moiety so the polymer and interaction molecule link via an azido-alkyne cycloaddition or via a strain-promoted alkyne-azido cycloaddition. Other polymers such as, PVA-, PAA-, HMPAA-, PPE- PLGA-based polymers can also be functionalized with an azide or alkyne moiety so they bind to the functionalized interaction molecule.

In some embodiments, the conjugates described herein contain a POx-based polymer functionalized with a diene moiety (e.g., pentadiene or cyclopentadiene) and an interaction molecule functionalized with a dienophile moiety (e.g., maleimide) so the polymer and interaction molecule conjugate via Diels-Alder reaction. Other polymers such as, PVA-, PAA-, HMPAA-, PPE- PLGA-based polymers can also be functionalized with a diene moiety so they bind to the functionalized interaction molecule.

### Interaction Molecules, Polymer/Interaction Molecule Conjugates, and Methods of Use

Any number of types of interaction molecules (e.g., proteins, non-protein organic molecules, inorganic molecules, etc.) may be conjugated to polymers set out herein. Further, conjugate methods, conjugation agents, and reactive groups used may vary with the polymers used, the attachment site(s) on these polymers, the interaction molecule(s), and the specific application.

Conjugation methods will generally be selected for one or more of the following reasons: (1) low toxicity (cellular and organismal), (2) amenability to desired degree of “decoration”, and (3) ease of use.

A number of groups associated with proteins, for example, may be used for polymer conjugation. These groups include (1) primary amines (R—NH₂), such as aliphatic amines and aromatic amines, (2) carbonyls (R—C═O), (3) thiols (or sulfhydryl groups, R—SH), and (4) carboxylic acids (R—COOH).

As used herein, “degree of decoration” refers to the percentage of conjugation sites of a polymer to which a molecule has conjugated. Further, degree of decoration may refer to one conjugation site type (e.g., carboxylic acids) or all conjugation site types (e.g., carboxylic acids and carbonyls). Also, degree of decoration may refer to groups inherent in a polymer or added to the polymer. By way of example, assume that a protein interaction molecule is conjugated to a polymer and maleimide groups are first added to carboxylic acid groups of the polymer and then protein is conjugated to the maleimide groups. In this instance, there would be two degrees of decoration. The first degree of decoration would be for the percentage of carboxylic acid groups present on the polymer to which maleimide groups have been conjugated. The second degree of decoration would be for the percentage of maleimide groups present on the polymer to which the protein has been conjugated. If the first and second degrees of decoration are both 90%, then the degree of decoration of the carboxylic acid groups of the polymer would be 81%. Unless stated otherwise, the degree of decoration, as used herein, refers to decoration of the groups original present in a polymer (e.g., 81% with respect to the carboxylic acid groups in the above example). Of course, higher orders of decoration (e.g., third, fourth, etc.) are also possible and will be determined by the number of conjugation components present between the polymer and the interaction molecule to which is they are conjugated to.

Degrees of decoration (of any type) may vary from about 20% to about 100% (e.g., from about 25% to about 100%, from about 30% to about 100%, from about 40% to about 100%, from about 55% to about 100%, from about 65% to about 100%, from about 80% to about 100%, from about 90% to about 100%, from about 95% to about 100%, from about 25% to about 95%, from about 30% to about 95%, from about 40% to about 95%, from about 55% to about 95%, from about 65% to about 95%, from about 80% to about 95%, from about 90% to about 95%, from about 55% to about 90%, from about 65% to about 90%, from about 80% to about 90%, etc.). The degree of decoration for any one polymer composition will vary with such factors as the polymer, the molecule conjugated to the polymer, and the reaction conditions (e.g., the pH, the respective concentration of the reactive groups and conjugation reagents).

Further, degrees of decoration will generally vary between decorated polymers within a single composition (e.g., the average degree of decoration) and decorated polymers generated by the same method (e.g., lot to lot variation). In each instance, one standard deviation for these variations will generally be 20% or less.

Interaction molecules that may be present in compositions provided herein (e.g., polymer-interaction molecule conjugates) include hormones (e.g., growth hormone, growth hormone releasing hormone, luteinizing hormone releasing hormone, pituitary hormone, thyroid hormone, male hormone, female hormone, epinephrine, amylin, gonadotropin, follicle stimulating hormone, parathyroid hormone, thymosins (such as thymosin alpha 1, thymosin beta 4, thymosin beta 9, thymosin beta 10, thymosin alpha 1, thymosin iib/iiia, etc.), 1-dihydrotestosterone, glucocorticoids, antidiuretic hormones, follicle stimulating hormone, bicalutamide, diethylstilbestrol, etc.); serum proteins (e.g. serum albumin, blood factors, blood coagulation factors (blood coagulation factors I, II, III, IV, V, VI, VII, VIIa, VIII, IX, X, XI, XII, XIII, von Willebrand factor, fibrinogen, etc.); cytokines and fragments (e.g., functional fragments) thereof, (e.g., interleukins (Interleukin-2, Interleukin-3, Interleukin-4, Interleukin-6, Interleukin-7, Interleukin-8, Interleukin-11, Interleukin-12, Interleukin-13, Interleukin-15, Interleukin-17, Interleukin-21, etc.)), interferons (e.g., Interferon-alpha, Interferon-beta, Interferon-gamma, Interferon-kappa, Interferon-omega, Interferon-tau, Interferon-lambda, Interferon-alpha-2 a, Interferon-alpha-2 b, Interferon-beta-1 a, Interferon-n 1, Interferon-n 3, Interferon-alpha 5, Interferon-gamma-1 b, consensus Interferon, etc.)), granulocyte colony stimulating factor, filgrastimulin, macrophage colony stimulating factor, granulocyte-macrophage, monocyte chemotactic proteins, platelet derived growth factors (platelet derived growth factors), thrombopoietin, phospholipase activator proteins, insulin, proinsulin, C-peptide, glucagon, insulin-like growth factors, insulin opsonins, glucagon-like peptides and analogs thereof (e.g., GLP-1, liraglutide, exendin, exenatide, Byduren, lixisenatide, loxapide, etc.), lectins, ricin, tumor necrosis factors (e.g., TNF-α), transforming growth factors (e.g., TGF-α, TFG-β, etc.), bone morphogenetic proteins (e.g., BMP-2, BMP-6, OP-1, etc.), osteoprotegerin, tissue growth factors, connective tissue growth factors, epidermal growth factors, hepatocyte growth factors, keratinocyte growth factors, endothelial growth factors, vascular endothelial growth factor, nerve growth factor, bone growth factor, insulin-like growth factor, heparin-binding growth factor, tumor growth factor, acidic fibroblast growth factor, basic fibroblast growth factor, glial cell line-derived neurotrophic factor, glial growth factor, macrophage differentiation factor, differentiation-inducing factor, leukemia inhibitory factor, amphiregulin, growth regulator, erythropoietin, neoerythropoiesis stimulating protein (NESP), hematopoeitin, angiotensin, calcitonin, elcatonin, lactoferrin, cystic fibrosis transmembrane conductance regulator, and the like); enzymes and corresponding zymogens (e.g., proteolytic enzymes, oxidoreductases, transferases, hydrolases, lyases, phenylalanine ammonia lyase, isomerases, ligases, aspartase, arginase, arginine deaminase, arginine deiminase, adenosine deaminase, deoxyribonuclease (e.g., deoxyribonuclease alpha), superoxide dismutase, endotoxases, catalase, chymotrypsin, lipase, uricase, elastase, streptokinase, urokinase, adenosine diphosphatase, tyrosinase, bilirubin oxidase, glucose oxidase, glucokinase, galactosidases (e.g., alpha-galactosidase, beta-galactosidase, etc.), glucosidases (e.g., alpha-glucosidase, beta-glucosidase, etc.), imiglucerase, arabinosidases, defibrase, plasmin, hyaluronidase, galactosidase, isomerase, beta-galactosidase, etc.); immunoglobulins (e.g., IgG, IgE, IgM, IgA, IgD, single-chain antibodies, variable heavy heavy chain antibodies (VHHs), etc.); monoclonal or polyclonal antibodies and fragments thereof, such as tumor necrosis factor alpha antibodies, GRO-beta antibodies, anti-CMV antibodies, anti-CD3 antibodies, anti-CD28 antibodies, anti-human interleukin-8 antibodies, anti-Tac antibodies, respiratory polysaccharide virus antibodies, abciximab, rituximab, trastuzumab, ibritumomab, tositumomab, alemtuzumab, gemtuzumab, cetuximab, bevacizumab, adalimumab, golimumab, basiliximab, infliximab, panitumumab, orvatuzumab, darlizumab, nimotuzumab, iodine [1311] mertuximab, belicantlizumab, ranibizumab, inotuzumab, obib, obizumab, ustikinumab, cetuximab, tuzumab, nimotuzumab, eduolimumab, edfumomozumab, ptolimumab, 3, rituximab, kumab, kumasuguamab, kumab, kumasuguakumab, kumab, kumasuguamab, kumab, dollomab aritox, isocromab pendentate, alefacept, abatacept, belatacept, aflibercept, Zinapax, abagodomab, abx-il8, actoumab, adecomumab, alirocumab, anifrolizumab, anti-LAG-3, apiolizumab, bapineuzumab, bavituximab, benralizumab, bertamumazumab, bispecific MDX-447, blinatumomab, blosozumab, braziumumab, brodalamuzumab, tunamuumumab ravitumumab ravatazumab, calamituzumab, caplatizumab, dolazumadurazumab, dolazulizumab, dolazululab, dolazulizumab, dolazululazululazululazulizumab, dolazulizumab, dolazululazululazululazululab, dolazululazululazulizumab, dolazulizumab, dolizumab, azulizumab, dolizumab, or tablet, or dolizumab, or tablet, olaratumab, olokizumab, onartuzumab, oregomomab, oteracib, palmitatuzumab, pembrolizumab, pemetroluzumab, pedelizumab, podilizumab, prolizumab, PRO 140, quilizumab, racitumomab, reslizumab, rilotuzumab, romumab, palmoluzumab, cantilizumab, palmitalizumab, salicylizumab, seculizumab, sevibuluzumab, sibutrumab, neturizumab, neturitab, tag, morolimumab, nacolomumafenatox, nebacterium, nerelimomab, odulimomab, ontuximab, oportuzumab monatx, orticimab, oxelumab, ozolalizumab, panobacunab, parsatuzumab, perazezumab, placitumab, priliximab (CMT 412), pertuzumab, radretazumab, rafivimab, regavirumab, robitumumab, rovelutimab/leuarest/Hu 23F2G, sazulimumab, sololizumab, suvivumab, tacitumomab texetan, taducizumab, talolizumab/TNX-901, tapolimus, thiotoxylmnate (e), thiotezomab (e), thiotezomib (99), heavy chain variable region such as a variable region of antibodies, such as heavy chain, variable region of antibodies, or fragment thereof); antagonists (e.g., growth factor antagonists, growth hormone antagonists, receptor antagonists, chemokine receptor antagonists, interleukin receptor-1 antagonist Rilonacept), antibody antagonists, kinase antagonists, and the like); non-protein drug molecules (e.g., flavonoids, terpenoids, carotenoids, saponins, steroids, quinones, anthraquinones, fluoquinones, coumarins, alkaloids, porphyrins, polyphenols, macrolides, monobactams, phenylpropanoid phenols, anthracyclines, aminoglycosides, and the like; and anti-Cancer or antineoplastic agents including, but not limited to, taxanes, paclitaxel and its derivatives, docetaxel, irinotecan, topotecan hydrochloride, topotecan, cisplatin, oxaliplatin, camptothecin and its derivatives, hydroxycamptothecin, vinblastine, vincristine, ipecacine hydrochloride, colchicine, doxorubicin, epirubicin, pirarubicin, valrubicin, doxorubicin or doxorubicin hydrochloride, epirubicin, daunorubicin, mitomycin, aclarubicin, idarubicin, bleomycin, pelomycin, daunorubicin, mithramycin, bleomycin, daunorubicin, rapamycin, disphramycin, streptozotocin, podophyllotoxin, actinomycin D (dactinomycin), maytansinoids, amikacin, mitoxantrone, all-trans retinoic acid, vindesicin, vinorelbine, and derivatives thereof, Gemcitabine, capecitabine, cladribine, pemetrexed disodium, tegafur, letrozole, anastrozole, fulvestrant, goserelin, triptorelin, leuprolide, buserelin, temozolomide, cyclophosphamide, ifosfamide, gefitinib, sunitinib, erlotinib, lapatinib, sorafenib, imatinib, dasatinib, nilotinib, sirolimus, everolimus, mercaptopurine, methotrexate, 5-fluorouracil, dacarbazine, hydroxyurea, vorinostat, ixabepilone, bortezomib, cytarabine, etoposide, azacytidine, teniposide, propranolol, procaine, tetracaine, lidocaine, besalbutadine, carmustine (dichloroethylnitrosourea), chlorambucil, methylbenzyl hydrazine, thiotepa, topotecan, erlotinib, and the like).

Polymer-interaction molecule conjugates may also be used to stimulate and/or activate immune cells (e.g., T cells) by blocking checkpoint inhibitors. Thus, interaction molecules include, for example, anti-CTLA-4 antibodies, anti-PD1 antibodies, anti-TIM-3 antibodies, and anti-LAG-3 antibodies. In some instances, checkpoint inhibitor antibodies, such as these, will be used in conjunction with other antibodies (e.g., anti-CD3 antibodies and anti-CD28 antibodies. Thus, provided herein are compositions comprising and methods employing antibodies that block checkpoint inhibitors. These antibodies may be used in free form or as components of Polymer-interaction molecule conjugates. By way of example, cells (e.g., T cells) may be contacted with a soluble antibody that block checkpoint inhibitor (e.g., an anti PD1 antibody) and polymer-interaction molecule conjugates that comprise anti-CD3 and anti CD28 antibodies.

Polymer-interaction molecule conjugates set out herein may be present in a number of different compositions and used in a number of different methods. Further, polymer-interaction molecule conjugates set out herein may be used in in vivo and/or ex vivo applications.

Interaction molecules used may result in induction of a cellular response (e.g., a receptor agonist) or inhibition of a cellular response (e.g., a receptor antagonist).

Polymer-interaction molecule conjugates that may be present in compositions and used in methods set out herein include polymers that may comprise one or more (e.g., from about 1 to about 40, from about 2 to about 40, from about 3 to about 40, from about 5 to about 40, from about 10 to about 40, from about 1 to about 30, from about 1 to about 20, from about 1 to about 10, from about 1 to about 5, from about 1 to about 3, from about 2 to about 10, from about 2 to about 5, from about 3 to about 20, from about 3 to about 10, from about 3 to about 6, from about 4 to about 10, etc.) interaction molecule.

In many instances, the starting point for polymer-interaction molecule conjugates design will be the desired use and specific conditions of use. As an example, if the application is treatment of allergic reactions in subjects, an anti-histamine (e.g., cetirizine) may be used as the interaction molecule. Further, the polymer-interaction molecule conjugate may be designed to not only deliver one or more interaction molecules to a target cell in a manner and in a local amount to exhibit an effect on target cells but can also be designed to have a half-life that allows for the maintenance of therapeutic effect with dosing at timed intervals (e.g., every 30 days).

When the application is ex vivo use, then the target cell (or cells) may be present in a culture medium. Further, the target cell (or cells) may be in isolated form (e.g., 100% of the total cell population) or non-target cells may be present. When target cells are purified from a sample obtained from a subject, at least some non-target cells will generally be present.

Some specific interaction molecules that may be present in compositions and used in methods set out herein include interferons (e.g., alpha, beta and/or gamma interferon), growth hormone, peptide hormones (e.g., luteinizing-hormone-releasing hormone, LHRH, etc.), interleukins, enzymes, antibodies, blood factors (e.g., GCSF, erythropoietin, Factor VIII, etc.), insulin, carbohydrates, oligonucleotides and small-molecule therapeutics such as anti-histamines, (e.g., cetirizine, desloratadine, etc.) and angiotensin receptor blockers (e.g., olmesartan, losartan, telmisartan, etc.).

Some of the applications of polymers set out herein are for cell activation, in particular immune cell activation. Immune cells that may be activated include monocytes, dendritic cells (DCs), natural killer (NK) cell and T cells.

Table 3 shows a number of the different T cell subtypes and signaling molecules that may be used to activate T cells of each cell type. CD3 and CD28 receptor stimulation are required for activation of most of the T cell types set out in Table 3. Further, interleukin-2 is also required for activation of a number of these T cell types. Thus, in some instances, polymer-interaction molecule conjugates may comprise CD3, CD28 and interleukin 2 receptor agonists. Further, such polymer-interaction molecule conjugates may comprise each individual subset of T cell activation signaling molecules, a subset of signaling molecules or all of the signaling molecules.

Issues for consideration when designing polymer-interaction molecule conjugates include the amount or relative amount of the interaction molecule conjugated to the polymer, which is important for interaction molecule “dosing”, also referred to as “signal strength” when used in reference to cell receptors. Thus, the total dosage of all interaction molecules, the total of individual interaction molecules, and relative doses of individual interaction molecules may be varied. Further, different T cell subsets have been shown to respond differently to differing amounts of receptor agonists for different cell receptors and for the same receptors. By way of example, it has been shown that a high ratio of stimulatory anti-CD3 receptor antibodies over stimulatory anti-CD28 receptor antibodies results in selective regulatory T cell (Treg) expansion over other cell types (US Patent Publication No. 2019/0062706A1).

With respect to T cells and T cell receptors, stimulation of these receptors can have a number of effects on particular T cell subtypes, as examples, (1) no effect upon the T cell subtype, (2) activation of the T cell subtype, (3) induction of proliferation of the T cell subtype, (4) polarization of the T cell subtype, (5) induction of differentiation of the T cell subtype (e.g., memory T cells), and (6) the induction of apoptosis in cells of the T cell subtype. The effect generated will often be a function of factors, such as the specific T cells present, the nature of the stimulatory signal(s), the ratio of the strength of multiple stimulatory signals (e.g., two, three, four, etc. signals) when multiple signals are employed, and the total or individual signal strength to which the T cells are exposed.

In many instances, T cells will be separated from other cell types prior to receptor stimulation. This may be done in a single step or in multiple steps. Exemplary methods are as follows: (1) buffy coat or apheresis isolation of mononuclear cells, (2) isolation of CD4+ cells using, for example, magnetic beads having one or more CD4 receptor binding agent, and (3) fluorescence activated cell sorting. Similar exemplary methods may be used for CD8+ and CD3+ cells.

In some aspects of compositions and methods provided herein, the ratio of two or more T cell signals are adjusted in a manner that results in selective expansion of a first set of one or more T cell subtype populations over a second set of one or more T cell subtype populations. In many instances, the first set of one or more (e.g., one, two, three, four, five, etc.) T cell subtype population will be smaller than the second set of one or more other T cell subtype populations. In some instances, the first set of one or more T cell subtype populations may comprise a single T cell subtype population and the second set of one or more T cell subtype populations may comprise all of the other T cell subtype populations present. In some instances, a first T cell subtype population (e.g., antigen experienced (memory) T cells) will be selectively expanded over a second T cell subtype population (e.g., naive T cells). Further, one or more additional T cell subtype populations may expand in conjunction with cells of the first T cell subtype population.

In many instances, one signal will be generated by stimulation of a first T cell receptor (e.g., the CD3 receptor) and another signal will be generated by stimulation of a second, co-stimulation T cell receptor (e.g., the CD28 receptor, the CD137 receptor, the CD27 receptor, the CD5 receptor, the CD6 receptor, the ICOS receptor, the CD134 receptor, etc.). Signal ratios may be altered in manner that (a) enhances the expansion of a particular T cell subtype population, (b) enhances the elimination of another T cell subtype population (e.g., via apoptosis, inhibition of cell growth, by having no expansion effect, etc.), or both (a) and (b). In some instances, one or more additional T cell receptors may also be stimulated or other signals may be provided to the T cells.

Exemplary ratios of stimulation signal of a first T cell receptor to stimulation signal of a second T cell receptor will vary with the T cell subtype population that is sought to be obtained and may be from about 50:1 to about 1:200 (e.g., about 1:5, about 1:10, about 1:15, about 1:20, about 1:40, from about 50:1 to about 1:40, from about 50:1 to about 1:30, from about 40:1 to about 1:40, from about 30:1 to about 1:40, from about 40:1 to about 1:20, from about 40:1 to about 1:10, from about 50:1 to about 1:1, from about 50:1 to about 5:1, from about 40:1 to about 5:1, from about 50:1 to about 10:1, from about 50:1 to about 15:1, from about 50:1 to about 20:1, from about 40:1 to about 5:1, from about 30:1 to about 3:1, from about 20:1 to about 3:1, from about 15:1 to about 3:1, from about 10:1 to about 5:1, from about 1:5 to about 1:10, from about 1:3 to about 1:20, from about 1:8 to about 1:25, from about 1:3 to about 1:40, from about 1:5 to about 1:50, from about 1:10 to about 1:50, from about 1:10 to about 1:100, from about 1:10 to about 1:150, from about 1:10 to about 1:200, from about 1:5 to about 1:150, from about 1:5 to about 1:200, from about 1:1 to about 1:20, from about 1:1 to about 1:15, from about 1:1 to about 1:10, from about 1:2 to about 1:20, from about 1:2 to about 1:10, from about 1:3 to about 1:10, etc.).

For purposes of illustration, signal provided by anti-CD3 antibodies and anti-CD28 antibodies may be present in a ratio of 1:10. It has been found that for expansion of some T cell subtype populations a lower amount of CD3 signal is desirable over a second signal (e.g., a CD28 signal and/or a CD137 signal). In some instances, when more than two T cell receptor signals are provided the ratio of each signal may be different or two or more of the signal ratios may be the same (e.g., two of three). As an example, CD3, CD28, and CD137 receptor signaling molecules may be present at a ratio of 1:10:10. When each of these signaling molecules are antibodies, this will generally mean that one part of an anti-CD3 antibody is present with ten parts of both anti-CD28 and anti-CD137 antibodies. This, of course, assumes that the amount of receptor stimulation is equal for each of the three receptors by their cognate antibody.

One issue for consideration is the composition of mixtures containing populations of T cells subtypes generated by methods provided herein. In some instances, compositions and methods provided herein will be directed to altering the ratio of T cells of particular subtype populations in mixtures. For example, methods provided herein may result in certain subtypes of T cells being eliminated from a mixed population by, as examples, apoptosis or dilution. Thus, one aspect of the compositions and methods provided herein relates to the amount of enhancement or depletion of a T cell subtype population in a mixture, as well as the mixtures themselves. For example, if there are two T cell subtype populations in a mixture (e.g., Th17 T cells and Th1 T cells) and these subtype populations are present in, for example, a 1:1 ratio, then provided herein are methods in which one T cell subtype population is increased in proportion to the other T cell subtype population. For purposes of illustration the ratio may be altered to from about 1:1.5 to about 1:100,000 (e.g., from about 1:1.5 to about 1:100,000, from about 1:1.5 to about 1:80,000, from about 1:1.5 to about 1:50,000, from about 1:1.5 to about 1:10,000, from about 1:1.5 to about 1:5,000, from about 1:2,500 to about 1:25,000, from about 1:2,500 to about 1:60,000, from about 1:2,500 to about 1:80,000, from about 1:2,500 to about 1:100,000, from about 1:5,000 to about 1:100,000, from about 1:5,000 to about 1:80,000, from about 1:5,000 to about 1:50,000, from about 1:5,000 to about 1:25,000, etc.).

Further, compositions and methods are provided herein for altering the ratio of T cells of a particular subtype populations in a mixture, where the proportion of one T cell subtype population is increased over another T cell subtype population by at least 200,000 fold (e.g., from about 1,000 fold to about 200,000 fold, from about 5,000 fold to about 200,000 fold, from about 10,000 fold to about 200,000 fold, from about 20,000 fold to about 200,000 fold, from about 50,000 fold to about 200,000 fold, from about 75,000 fold to about 200,000 fold, from about 1,000 fold to about 120,000 fold, from about 5,000 fold to about 120,000 fold, from about 10,000 fold to about 120,000 fold, from about 1,000 fold to about 80,000 fold, from about 10,000 fold to about 80,000 fold, etc. An example of what is meant by “fold” is illustrated as follows. If two T cell subtype populations are present in an initial ratio of 1:2, then an alteration in their ratio to 1:8 is a 4 fold increase of one T cell subtype population with respect to the other T cell subtype population.

In some instances, fold expansion will be determined at specific time intervals. Thus compositions and methods are provided herein for an increase in the number or total T cells or a subpopulation of T cells four, six or eight days after expansion where the fold expansion is from about 4 to about 100 (e.g., from about 4 to about 90, from about 4 to about 80, from about 4 to about 70, from about 4 to about 60, from about 4 to about 50, from about 4 to about 40, from about 4 to about 30, from about 6 to about 100, from about 6 to about 80, from about 6 to about 65, from about 6 to about 55, from about 6 to about 45, from about 6 to about 35, from about 8 to about 100, from about 8 to about 40, from about 8 to about 30, from about 8 to about 20, from about 8 to about 15, from about 9 to about 50, from about 9 to about 35, from about 9 to about 25, etc.).

In some instances, the percent of live/viable cells in populations of T cells activated by methods set out herein that express CD69 receptors and/or CD25 receptors will be from about 50% to about 100% (e.g., from about 50% to about 100%, from about 60% to about 100%, from about 70% to about 100%, from about 80% to about 100%, from about 90% to about 100%, from about 93% to about 100%, from about 80% to about 98%, from about 90% to about 98%, from about 90% to about 96%, etc.). Typically, CD69 receptor and/or CD25 receptor expression will be measured at day one, two, three, or four after contact with polymer-interaction molecules (e.g., G5-anti-CD3 VHH (G5-CD3) and G5-anti-CD28 VHH (G5-CD28) polymer-interaction molecules).

Another factor that can result is the selective expansion of individual T cell subtype populations is stimulus signal strength. By “stimulus signal strength” refers to the total signal strength on a per T cell basis. This includes the strength of the various signals (e.g., a signal stimulating a first T cell surface receptor, a signal stimulation of a second T cell surface receptor, a signal stimulation of a third T cell surface receptor, etc.) and the combined signal to which each T cell in the population is exposed to. Thus, compositions and methods provided herein also relate to the amount of stimulatory signal received by each cell in a mixture of various T cell subtype populations. The stimulatory signal can be modulated by alterations to concentrations of stimulatory agents, ratios thereof, or ratios of polymer-interaction molecule conjugates to cell count.

In many instances, the number of interaction molecules conjugated to each polymer molecule will vary with factors such as the size of the interaction molecules, the size of the polymer and the number of conjugation points on the polymer. One factor that may affect the number of interaction molecules that can be conjugated to a polymer molecule is steric hinderance. Thus, in many instances, the larger the interaction molecule, the more spaced out the individual interaction molecules will be and the fewer interaction molecules there will be on the polymer.

The number of interaction molecules conjugated to each polymer molecule may vary and includes from about 1 to about 500 (e.g., from about 1 to about 400, from about 1 to about 300, from about 1 to about 200, from about 1 to about 100, from about 1 to about 50, from about 1 to about 30, from about 1 to about 20, from about 1 to about 10, from about 2 to about 400, from about 2 to about 200, from about 2 to about 100, from about 2 to about 50, from about 2 to about 25, from about 2 to about 10, from about 3 to about 10, from about 3 to about 25, from about 3 to about 40, from about 4 to about 12, from about 4 to about 25, from about 4 to about 50, from about 6 to about 400, from about 6 to about 130, from about 6 to about 75, from about 6 to about 25, from about 6 to about 18, from about 7 to about 25, from about 7 to about 50, from about 7 to about 100, from about 8 to about 30, from about 10 to about 25, from about 10 to about 75, etc.).

In some instances, one or more cytokine may be added to a cell population (e.g., a T cell population). In many instances, IL-1 beta, IL-2, IL-4, IL-5, IL-6, IL-7, IL-10, IL-12, IL-13, IL-15, IL-21, IL-23, IFN-gamma, and TGF-beta. One of more of these cytokines may be a component of a polymer-interaction molecule conjugate. By way of example, when Th17 polarization is desired, one or more of the following cytokines may be used: IL-1β, IL-6, TGF-β, IL-21, IL-23, and neutralizing anti-IL-4 and anti-IFN-gamma antibodies. Cells may be contacted with all of these proteins, as well as other interaction molecules, in unconjugated form or as polymer-interaction molecule conjugates. Further, as examples, cells may be contacted with IL-1β and IL-6 where both of these interleukins are conjugated to the same polymer molecule or different polymer molecules. Additionally, one cytokine (e.g., IL-1β) may be conjugated a polymer molecule and the other cytokine may be contacted with cells unconjugated (e.g., in soluble form).

TABLE 3
T Cell Subtype Stimuli

| | Signal 1 | Signal 2 | |
|---|---|---|---|
| T cell Subtype | (Agonists) | (Agonists) | Signal 3% |
| Naïve T cells | CD3 | CD28 | IL-2 |
| Central | CD3 | CD28 | IL-2, IL-7, |
| Memory T cells | | | IL-15 |
| Effector | CD3 | CD137 and/or CD27 | IL-2, IL-15, |
| Memory T cells | | and/or CD28 and/or other | IL-7, IL-21 |
| Treg | CD3 | CD5, CD28, and/or | IL-2 |
| | | CD278 (ICOS) | |
| Th17 | CD3 | CD5, CD6, and/or | IL-21, TGFβ, |
| | | CD278 (ICOS) | IL-6, IL-1beta |
| Th2 | CD3 | CD28 | IL-4 |
| Th1 | CD3 | CD28 | IL-2, IL-12 |
| Th22 | CD3 | CD28 and/or | IL-23, IL-6 |
| | | other | |
| Th9 | CD3 | CD28 and/or | IL-4, TGFβ |
| | | other | |

*“and/or” in each instance where more than an agent is set out in this column.

Additionally provided herein are compositions and methods for the selective expansion of one or more T cell subtype populations. Such methods result in the enhancement or depletion of specific T cells in a sample. As an example, naïve T cells, memory T cells, Th1 T cells and regulatory T cells (Tregs) stimulation of CD3 and CD28 receptors in conjunction with Interleukin-2. It has been shown that naïve T cells may be expanded while memory T cells may be depleted from a sample by the adjustment of total CD3/CD28 stimulus (see U.S. Pat. No. 8,617,884). It has now been found that different T cell subtype populations present in a mixed population may be selectively expanded by adjusting signal ratios and total signal strength (see PCT Publication WO 2017/072251 A1). As an example, Treg cells expand well when CD3 signal is lower than CD28 signal (see US Patent Publication No. 2019/0062706A1). The identification of selective expansion conditions can be used to increase the proportion of members of one T cell subtype population over member of one or more other T cell subtype populations in a sample, even when the various cells of the various T cell subtype populations expand in response to the same stimuli. For purposes of illustration, assume that Treg T cells represent 1% of a mixed population and naïve T cells, memory T cells are represent, respectively, 1.5%, 3% of the same mixed population, stimulatory signals may be adjusted to induce elimination of memory T cells, while selectively expanding Treg T cells. The net result may be a mix population where Treg T cells represent 40% and naïve T cells, memory T cells, and Th1 T cells are present, respectively, 2%, 0.5% and 2.5% of the mixed population.

An additional agent that may be used for the selective enhancement or depletion of one or more T cell subtypes (e.g., CD4+CD25+FOXP3+ regulatory T cells, CD4+CD25+FOXP3− regulatory T cells, CD4+CD25− T cells, etc.) is rapamycin.

Polymer-interaction molecule conjugates provided herein include those that comprise one or more of the following monoclonal antibodies: Anti-CD3 antibody BC3 (BioLegend, cat. no. MMS-5212), anti-CD28 antibody CD28.6 (Thermo Fisher Scientific, cat. no. 16-0288-81), Anti-ICOS antibody ISA-3 (Thermo Fisher Scientific, cat. no. 14-9948-82), anti-CD5 antibody from clone UCHT2 (Thermo Fisher Scientific, cat. no. 12-0059-42). Polymer-interaction molecule conjugates provided herein include those that comprise one or both of the following single domain antibodies: Receptor Activation Anti-CD3 VHH antibody and Receptor Activation Anti-CD28 VHH antibody.

Interaction molecule dosing may be achieved in a number of ways. When a single interaction molecule (e.g., cetirizine) is used, dosing relates to the amount of that interaction molecule per number of individual cells. Further, the number of interaction molecules per polymer of the polymer-interaction molecule conjugate determines the amount of polymer-interaction molecule conjugate(s) to be used on per cell basis. Other variables include (1) the number of cellular molecules (e.g., receptors) available for interaction molecule interaction, (2) the number of cellular molecules (e.g., receptors) that for interaction molecules must bind to in order to result in the desired effect (e.g., cell activation), and (3) the nature of the desired cellular response (e.g., “all or none”, graded, etc.).

The number of interaction molecules and the number of polymer-interaction molecule conjugates brought into contact with cells on a per cell basis may vary greatly. In some instances, the number of interaction molecules of a single type (e.g., an anti-CD3 antibody) or the number of polymer-interaction molecule conjugates brought into contact with cells on a per cell basis will be from about 400 to about 10,000 (e.g., from about 1,000 to about 10,000, from about 2,000 to about 10,000, from about 4,000 to about 10,000, from about 6,000 to about 10,000, from about 8,000 to about 10,000, from about 1,000 to about 8,000, from about 1,000 to about 6,000, etc.).

When an interaction molecule mediates an effect on cells, then the amount of interaction molecule present will typically be enough to mediate the effect in at least 75% of the cells (e.g., from about 75% to about 100%, from about 80% to about 100%, from about 85% to about 100%, from about 90% to about 100%, from about 95% to about 100%, from about 75% to about 98%, from about 80% to about 98%, from about 85% to about 98%, from about 90% to about 98%, from about 75% to about 95%, from about 80% to about 95%, from about 85% to about 95%, from about 75% to about 90%, from about 80% to about 90%, from about 82% to about 92%, etc.).

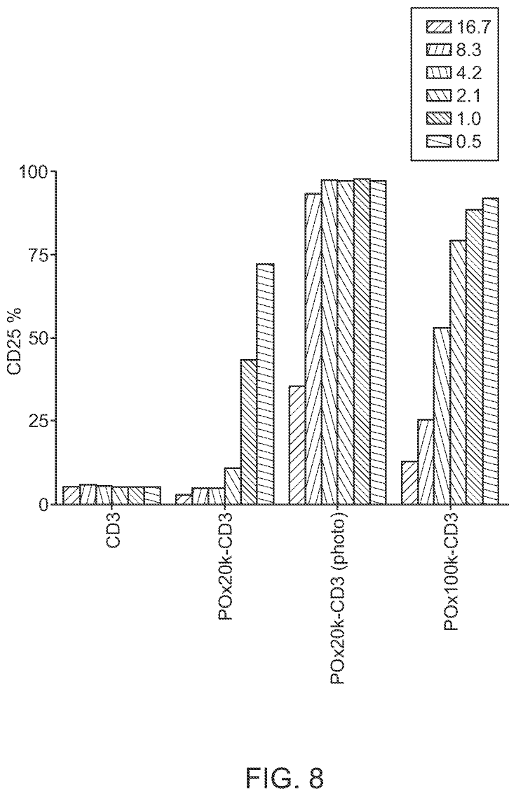

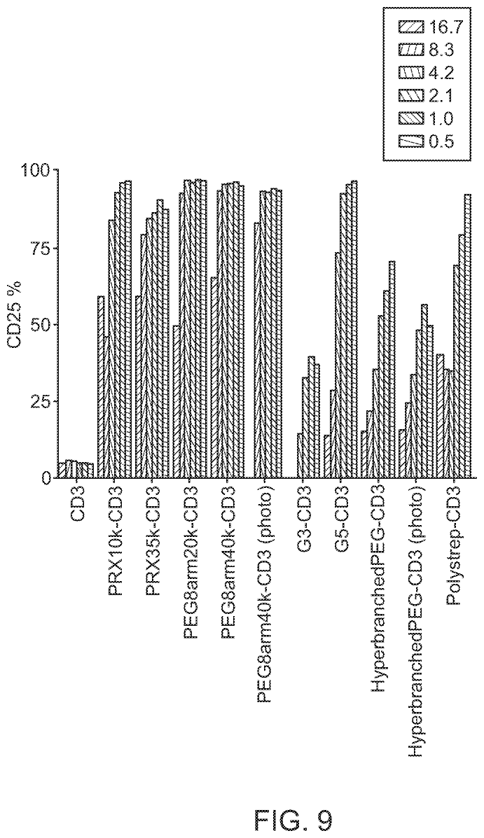

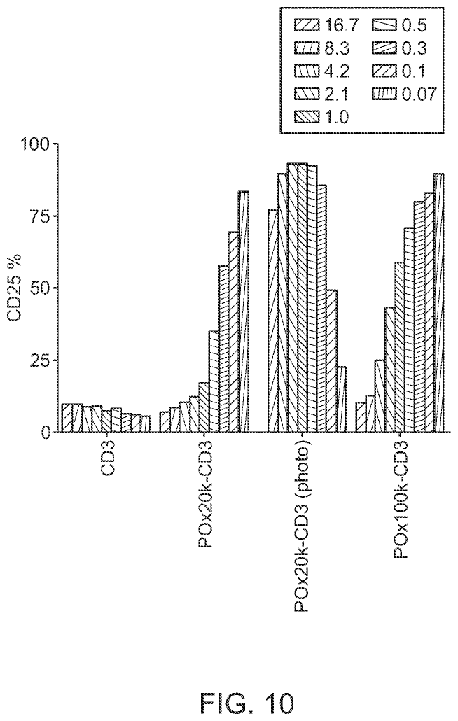

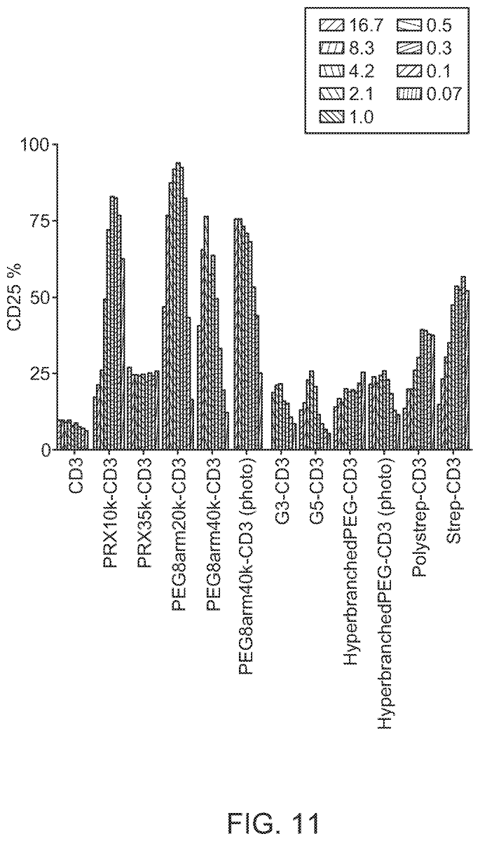

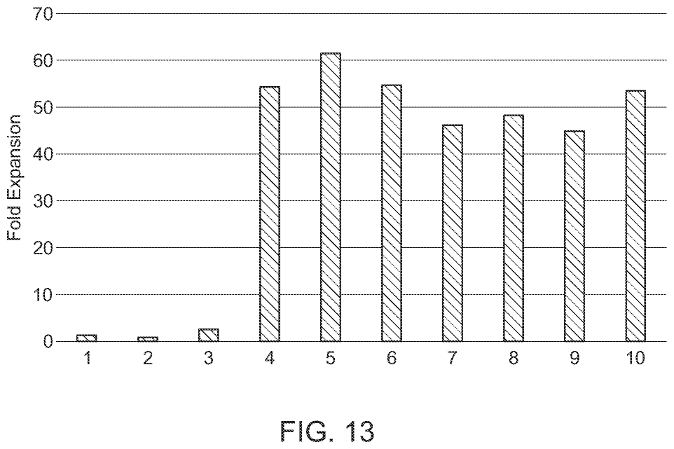

An example of a cellular effect is when T cells are stimulated with a CD3 receptor agonist alone, the cells express CD25 receptors (see FIGS. 8-11). Another example of a cellular effect is when T cells are stimulated with a CD3 receptor agonist and a CD28 receptor agonist, the cells proliferate (see FIG. 13). Each of these is an effect that can be readily measured.

In many instances, cells will be contacted with polymer-interaction molecule conjugates ex vivo. In some instances, this will be done prior to introduction of the cells into a subject. Thus, in some instances, the contacting of cells with polymer-interaction molecule conjugates will be part of a larger workflow or process.

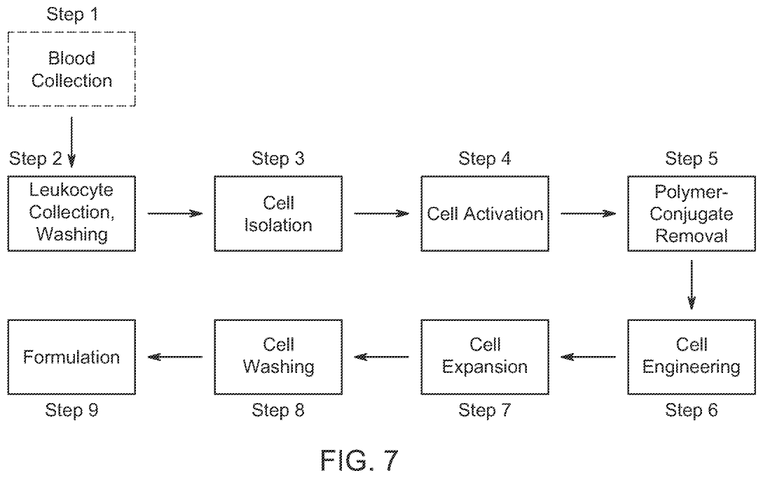

FIG. 7 is a schematic of exemplary workflows provided herein. The steps of the schematically represented workflow set out in FIG. 7 can be performed where cells are treated under different conditions at different time points and, in some instances, using different types of equipment. Thus, provided herein are modular cell processing workflows in which cells may be processed, for example, as a series of stations and/or using two or more different instruments.

Further, in some instances, the order of steps set out in FIG. 7 may be altered and some steps may be repeated consecutively or in another location in the workflow. As examples, in some instances, some workflows may involve performing the following steps in sequences: (A) Step 3, Step 4, Step 5, Step 6, and Step 7; (B) Step 3, Step, 5, Step 6, and Step 7; or (C) Step 3, Step 4, Step 5, Step 6, and Step 8. Further, due to the length of time cells are activated, Step 3 and Step 4 may overlap or be one in the same. This is so because, in many instances, cells begin to expand once activated. Further, cells may remain in contact with activating stimuli for an extended period of time. This will often be the case when activation stimuli (e.g., anti-CD3 and anti-CD28 antibodies) remain in contact with cells until Step 5. In some instances, Step 5 will be omitted.

Further, some of all of the steps set out in FIG. 7 may be performed as a closed system. By closed system is meant that cells remain in closed vessels during a single process or multiple processes. For example, in Step 1 through Step 5, cells may be transferred between bags but the interior of each bag may be a sterile environment and the cells may be transferred from bag to bag using sterile tubing and connectors. In such an instance, Steps 1 through Steps 5 would be considered to be a closed system and would further be considered to be a sterile, closed system. In some instances, Step 2 through Step 9 (or a subset of such steps) will be performed in a closed system.

In many instances, workflows set out herein will be directed to the generation of CAR-T cell populations.

The first step in workflows of FIG. 7 (Step 1) is the collection of blood from an individual (e.g., a patient). This individual may not be in need of therapeutic treatment related to the blood collection. Alternatively, this individual may be, for example, afflicted with a condition for which the treatment of involves administration of the formulation of Step 9. The blood obtained from the individual may be processed, for example, by leukapheresis where the blood is removed from the individual's body, the leukocytes are collected, and the uncollected blood components are returned to the individual. An instrument that may be used for PBMC isolation from whole blood is the CELL SAVER® 5+ Autologous Blood Recovery System (Haemonetics Corporation, Boston, MA).

Once the desired number of leukocytes have been harvested, the resulting cell population is generally washed (Step 2) to remove, for example, anti-Coagulant(s). In an early step, the cell population may be enriched for lymphocytes (Step 2) using, for example, a counterflow centrifugal elutriation system (e.g., a GIBCO™ CTS™ ROTEA™ Counterflow Centrifugation System, Thermo Fisher Scientific), which can separate cells by size and density.

Isolation of desired cell types (Step 3) (e.g., total T cell and T cell subsets, CD34+ stem cells, natural killer cells, as well as other cell types), may be performed using ligands having binding affinity for cell surface receptors. Examples of such cell surface receptors include CD3, CD4, CD5, CD6, CD8, CD25, CD27, CD28, CD137, and CD278 (ICOS). Further, isolation and activation may occur simultaneously. As an example, a mixed population of leukocytes may be exposed to anti-CD3 and anti-CD28 antibodies under conditions in which T cells are separated from other leukocytes and the combination of the anti-CD3 and anti-CD28 antibodies results in T cell activation.

By way of specific example, T cells may be isolated based upon the presence on their surfaces of CD3 markers. Some isolation methods use positive isolation of cells with the desired surface marker. An exemplary method for T cell isolation is as follows. A mixed leukocyte population is incubated (e.g., 20-30 minutes at 4° C.) with magnetic beads with anti-CD3 antibodies located on the bead surfaces (e.g., DYNABEADS® CD3, Thermo Fisher Scientific, cat. no. 11151D) for sufficient time for the beads to associate with T cells in the population. In many instances, such anti-CD3 antibodies will not stimulate CD3 receptors of cells to which they bind. The cells may then be contacted with a magnetic field under conditions that allow for cells bound to the beads to be retained while cells not bound to the beads to be removed (e.g., by washing). This results in the separation of T cells from non-T cells of the leukocyte population.

In many instances, once T cells have been isolated, these cells will be contacted with an anti-CD3 antibody capable of stimulating CD3 receptors and/or an anti-CD28 antibody capable of stimulating CD28 receptors, resulting in T cell activation. Either one or both of these anti-CD3 and anti-CD28 antibodies may be components of one or more polymer-interaction molecule conjugate.

T cells exposed to anti-CD3 antibodies and/or anti-CD3 and anti-CD28 antibodies may be analyzed for activation levels. One type of assay for measuring activation is based upon screened T cells for CD25 (the alpha chain of the IL-2 receptor) expression levels. While the CD25 marker is found on a number of peripheral blood lymphocytes (e.g., regulatory and resting memory T cells), CD25 expression is generally considered to be a prominent T cell activation marker. Thus, methods provided herein include methods for measuring the percentage of activated T cells in a population. This percentage is calculated by comparing the number of non-activated T cells with the number of activated T cells. Of course, the percentage of activated T cells will change with such factors as the duration of exposure to activation signals and as activated T cells expand.

Step 4 in the exemplary workflow of FIG. 7 is cell activation and expansion. During this step, cells will often be exposed with activation signals for an extended period of time (e.g., from about 1 day to about 20 days, from about 2 days to about 20 days, from about 4 days to about 20 days, from about 4 days to about 15 days, from about 4 days to about 14 days, from about 6 days to about 14 days, etc.).

While expansion conditions may vary conditions, activated T cells may be cultured, for example, at 37° C. and 5% CO₂ in cell culture medium (e.g., CTS™ OPTANZER™ media without phenol red plus 2-5% CTS™ Immune Cell SR (Thermo Fisher Scientific, cat. nos. A3705001 and 15710-049). Further, cytokines and fresh medium may be added every 1-3 days to maintain a cell concentration of 0.5-2×10⁶ cells/ml. T regulatory cells (Tregs) may be expanded in medium containing 100 ng/ml rapamycin (e.g., Thermo Fisher Scientific, cat. no. PHZ1235) and 300 IU IL-2/ml (e.g., Thermo Fisher Scientific, cat. no. PHC0027). CMV stimulated T cells may be expanded in 100 IU IL2/ml. Th17 cells may be expanded in medium containing polarizing cytokines (IL-6, IL-13, IL-23, and TGF-13, all, for example, from Thermo Fisher Scientific CA USA) in presence of anti-IL-4 and anti-IFN-γ neutralizing antibodies (both, for example, from Thermo Fisher Scientific, CA US) as described in Paulos et al. (Paulos et al., Science Transi. Med 55:55ra78 (2010)). Further, 100 IU IL-2/ml may be added day 3 post-activation. IL-2 may also be added one day 0, for example, during dilution of cells from the isolation bag to the output.

Expansion of cells (Steps 4 and 7) will generally occur under conditions suitable for cell division. Media that may be using for expansion include CTS™ OPTMIZER™ T-Cell Expansion SFM (Thermo Fisher Scientific, cat. no. A3705001) and LYMPHOONE™ T-Cell Expansion Xeno-Free Medium (Takara Bio, cat. no. WK552S).

In some instances, it may not be necessary or desirable to separate polymer-interaction conjugates from cells. This may be so due to the low toxicity of polymer-interaction molecule conjugates used and because the concentration of polymer-interaction conjugates on a per cell basis will decrease as T cells expand. In such instances, Step 5 set out in FIG. 7 may be omitted.

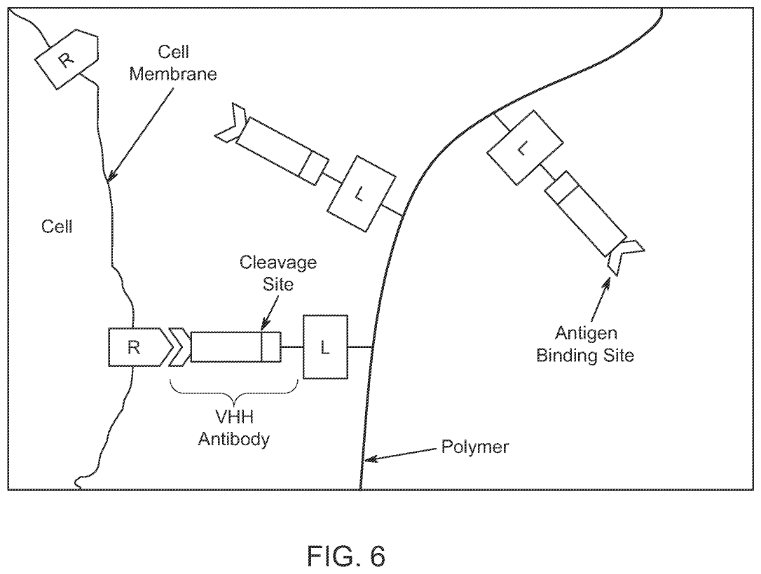

In some instances, it will be desirable at some point in workflow to remove polymer-interaction conjugates from contact with cells. In some instances, when the supports are bound to the cells, it may be necessary or desirable to disrupt the binding of the supports to the cells. For example, the cells and the polymers may be associated with each other through conjugation of antibodies to the polymers. FIG. 6 shows a schematic of a cell bound to a polymer. A VHH antibody is shown in FIG. 6 bound to a cell surface receptor (e.g., a CD3, CD4, CD8, CD11a, CD11b, CD14, CD15, CD16, CD19, CD20, CD22, CD24, CD25, CD28, CD30, CD31, CD34, CD38, CD45, CD56, CD61, CD91, CD114, CD117, CD182, etc.), labeled “R”. Further, the cell type(s) bound by the antibodies may be one or more of any number of cell types (e.g., stem cells, leukocytes in general, granulocytes, monocytes, total T cells, helper T helper cells, regulatory T cells, cytotoxic T cells, B cells, natural killer cells, dendritic cells, thrombocytes, etc.).

Disruption of association of polymers from cells may be accomplished by a number of means. An exemplary cell release features are represented in FIG. 6 where a cleavage site is shown in the antibodies that allow for disruption of antibody association with the cells and/or the support. In some instances, the disruption of antibody association may be based upon cleavage of the antibody into different parts where one part contains the antigen binding domains and another part is associated with the support. An exemplary cleavage mechanism involves antibody cleavage. Antibody cleavage may be mediated, for examples, by naturally occurring protease cleavage sites or protease cleavage sites that have been introduced into the antibodies. Proteases that may be used include a tobacco etch virus (TEV) protease, a TEV protease with an S219V modification (e.g., AcTEV™ Protease, Thermo Fisher Scientific, cat. no. 12575015), a rhinovirus 3C protease, a TVMV protease, a plum pox virus protease, and a turnip mosaic virus protease, enteropeptidase, thrombin and Factor Xa. Thus, methods set out herein include those where cells and supports are dissociated from each other by cleavage mediated by one or more protease. Also included herein are compositions for performing such methods (e.g., antibodies engineered to contain one or more protease cleavage site). Some methods related to the above are set out in U.S. Patent Publication No. 2017/0313772 A1.

One process that may be employed for the dissociation of cells and polymers makes use of anti-biotin antibodies. For example, a two antibody linking systems can be used where a first biotinylated antibody is used wherein the first antibody has binding affinity for a cell surface protein (e.g., a receptor). A second anti-biotin antibody may be conjugated to a polymer. Thus, the cells are associated with the polymer, in part, through the binding of the binding of the polymer bound second antibody (anti-biotin antibody) to the first antibody (biotinylated, anti-cell surface protein antibody). Disruption of association between the polymer and cells may be mediated by disruption of the binding of the second antibody to the biotin of the first antibody. This may be accomplished by contacting the cell/polymer complex with a releasing agent (e.g., biotin or biotin derivative). Compositions and methods related to the above are contained in U.S. Pat. No. 10,196,631.

In many instances, materials bound to cells release after a period of time. With T cells this is believed to be a result of down-regulation of the cell surface marker bound to the antibody. Thus, in many instances, cells may be separated from polymers without the performance of an active dissociation step. In many such instances, separation of cells from polymers will occur after cells (e.g., T cells) have been expanded for from about 4 to about 21 days (e.g., from about 4 to about 21, from about 5 to about 21, from about 6 to about 21, from about 5 to about 14, from about 5 to about 12, from about 5 to about 10, from about 6 to about 14, from about 6 to about 12, from about 6 to about 10, etc. days). Also in many such instances, separation of cells from polymers will occur after cells (e.g., T cells) after greater than 70% (e.g., from about 70% to about 99%, from about 70% to about 98%, from about 70% to about 95%, from about 70% to about 90%, from about 70% to about 85%, from about 75% to about 99%, from about 80% to about 99%, from about 85% to about 99%, from about 85% to about 95%, from about 85% to about 90%, from about 90% to about 99%, from about 90% to about 97%, etc.) of the cells are dissociated from polymers.

Removal of polymers and polymer-interaction molecule conjugates may be performed in any number of places in workflows but may be performed as part of Step 5 and in Step 8 of FIG. 7. Efficiency of separation of polymers and interaction molecules from cells may be measured by the use of fluorescent dyes. For example, one or more fluorescent dye may be conjugated to polymers and polymer-interaction molecule conjugates and fluorescence-activated cell sorting (FACS) may be used to determine residual fluorescence after cells have been washed.

In some instances, one or more fluorescent dye may be conjugated to the polymer component of a polymer-interaction molecule conjugate. In some instance, one or more fluorescent dye may be conjugated to an interaction molecule component of a polymer-interaction molecule conjugate. Further, two such polymer-interaction molecule conjugates may be used in conjunction with each other. When the fluorescent dyes of the polymer and interaction molecule are different, it is possible to separately detect residual polymer and interaction molecules associated with cells after washing.

Fluorescent dyes that may be present in compositions and used in methods set out herein include, for example, fluorescein, rhodamine, tetramethylrhodamine, OREGON GREEN™, the ALEXA-FLUOR® dyes (e.g., ALEXA-FLUOR® 350, ALEXA-FLUOR® 430, ALEXA-FLUOR® 488, ALEXA-FLUOR® 546, ALEXA-FLUOR® 568, ALEXA-FLUOR® 594, ALEXA-FLUOR® 633, ALEXA-FLUOR® 647, ALEXA-FLUOR® 660, ALEXA-FLUOR® 680, ALEXA-FLUOR® 700, ALEXA-FLUOR® 750), CASCADE BLUE™, and R-phycoerythrin, all of which are available from Thermo Fisher Scientific.

Step 6 set out in FIG. 7 is cell engineering. In some instances, this step will not be performed. Further, when this step is performed, it may differ greatly for the cell type being engineered and the purpose of the cell engineering.

T cells, for example, may be engineered to expression chimeric antigen receptors (CARs). CARs are receptors that are designed to bind to cell surface proteins on target cells (e.g., human leukocyte antigen antigens. Further, T cells may be engineered to express CARs on their surface, allowing them to recognize specific antigens (e.g., tumor antigens). These CAR T cells can then be expanded by methods provided herein and infused into the patient. Typically, this will occur after the T cells are washed (Step 8 in FIG. 7) and formulated for patient administration (Step 9 in FIG. 7).

In some instances, cells (e.g., a T cell) may be engineered to express a CAR wherein the CAR T cell exhibits an antitumor property. CARs can be designed to comprise an extracellular domain having an antigen binding domain fused to an intracellular signaling domain of the T cell antigen receptor complex zeta chain (e.g., CD3 zeta). Such a CAR, when expressed in a T cell is able to redirect antigen recognition based on the antigen binding specificity.

Polymer-interaction molecule conjugates include those that may be used to stimulate natural killer (NK) cell expansion. These include polymer-interaction molecule conjugates comprising interleukin-2, interleukin-10, interleukin-15, and/or interleukin-21, as well as methods of using such polymer-interaction molecule conjugates for the expansion of NK cells.

Engineering NK cells may be engineered to express a chimeric antigen receptor (CAR) to generate CAR NK cells. Engineering NK cells may be desirable when one seeks to “target” cells having one or more receptors (e.g., CD19 receptors).

Some of the advantages of NK cells for cancer immunotherapy are as follows. First, in general, NK cells are allogenic and, thus, cause little to no graft vs. host disease (GVHD). Additionally, the cytokine levels generated by infusions of NK cell infusions are generally lower than those found for CAR T cell infusions. Further, NK cells have a relatively short circulation half-life (˜714 days). Also, NK cells can be generated from cord blood and cell lines.

It has been found that different sets of stimuli may be used to induce NK cell expansion. One commercially available product that contains reagents and protocols for ex vivo NK cell expansion is available from BIO-TECHNE® (CLOUDZ™ Human NK Cell Expansion Kit, cat. no. CLD004). In the process used in this product, NK cells are contacted with agonistic antibodies targeting CD2 and NKp46 (CD335) receptors. Further, the cells are also contacted with the cytokines IL-2, IL-12, IL-18, and IL-21.

Another commercially available product that contains reagents and protocols for ex vivo NK cell expansion is available from Miltenyi (NK Cell Activation/Expansion Kit, cat. no. 130-094-483). In the process used in this product, NK cells are contacted with agonistic antibodies targeting CD2 and NKp46 (CD335) receptors. Further, the cells are also contacted with the cytokine IL-2.

Several different protocols for induction of ex vivo NK cell expansion are set out in Spanholtz et al., “High Log-Scale Expansion of Functional Human Natural Killer Cells from Umbilical Cord Blood CD34-Positive Cells for Adoptive Cancer Immunotherapy”, PLoS One, 5:e9221 (2010). In particular, Spanholtz et al., sets out several protocols for the generation of NK cells from hematopoietic stem and progenitor cells obtained from umbilical cord blood. In these protocols, cells were cultured in media containing 10% human serum and contacted at different times with different mixtures of the following cytokines: granulocyte-macrophage colony-stimulating factor (GM-CSF), granulocyte colony-stimulating factor (G-CSF), leukemia inhibitory factor (LIF), macrophage inflammatory protein 1α (MIP1α), stem cell factor (SCF), FMS-like tyrosine kinase 3 ligand (F1t3L), thrombopoietin (TPO), IL-2, IL-6, IL-7, and IL-15. In one protocol set out in Spanholtz et al., CD34⁺ cells are contacted with SCF, IL6, IL-7, TPO, G-CSF, and GM-CSF from days 0 to 9; then with SCF, IL-6, IL7, IL-15, TPO, G-CSF, and GM-CSF from days 9 to 14; and then with SCF, IL-2, IL6, IL-7, IL-15, G-CSF, and GM-CSF from days 14 to 42.

Many methods for the expansion of NK cells use one or both IL-15 or IL-21, or IL-15 or IL-21 agonists. NK cell expansion may also be mediated by the stimulation of NKp46, CD2, CD16, MICA/B, and CD137 receptors. While IL-15 alone is capable of inducing NK cell expansions, in many instances, a combination of stimulatory signals/molecules are used. Thus, composition provided herein include polymer-interaction molecule conjugates comprising IL-15, one or more IL-15 agonist, IL-21, one or more IL-21 agonist, one or more NKp46 receptor agonist, one or more CD2 receptor agonist, one or more CD16 receptor agonist, one or more MICA/B receptor agonist, and/or one or more CD137 receptor agonist. Further, one or more of these NK cell stimulatory agents may be conjugated to the same or different polymers. Also, provided herein are methods for inducing NK cell expansion in which NK cells are contacted with one or more of the above NK cell stimulatory agents. By way of example, provided herein are methods for inducing NK cell expansion where the NK cells are contacted with agonistic antibodies targeting CD2 and NKp46 receptors conjugated the same or different polymers and IL-15 cytokine in unconjugated form.

Provided herein are compositions and methods for generating NK cells and/or inducing proliferation of NK cells. Polymer-interaction molecule conjugates used in such methods may comprise one or more anti-CD2 receptor antibodies (e.g., agonistic anti-CD2 receptor antibodies), anti-NKp46 receptors (e.g., agonistic anti-NKp46 receptor antibodies), SCF, GM-CSF, G-CSF, LIF, MIP1α, SCF, Flt3L, TPO, IL-2, IL-6, IL-7, IL-12, IL-15, IL-18, and IL-21. In some instances, one or more of these interaction molecules (as well as other interaction molecules) may be conjugated to the same polymer. In other instance, polymers will each contain only one or these interaction molecules. Further, when more than one interaction molecule is conjugated to a polymer, then the interaction molecules may be present at the same amount or different amounts. The terms “amount” and “amounts” in this count refer to the number of interaction molecules.

One advantage of using polymer-interaction molecule conjugates that contain only a single interaction molecule are that this allows for cells to be contacted with different amounts of interaction molecule without the need for reformulating the polymer-interaction molecule conjugates used. Another advantage is that it allows for the use of interaction molecules in different combinations, again, without the need for reformulating the polymer-interaction molecule conjugates used. These principles apply to all polymer-interaction molecule conjugates provided herein.

Provided herein are compositions and methods for enhancing the expansion of one or more cell types while inhibiting the expansion of one or more other cell types. As an example, regulatory T cells (Tregs) may be selectively expanded over other T cells by exposing a mixed population of T cells to lower CD3 receptor signal in relation to higher CD28 receptor signal (see PCT Publication WO 2017/072251). As another example, naïve T cells may be expanded in a mixed population under conditions in which memory T cells are deleted from the population, presumably by apoptosis by exposing the CD3 and CD28 receptors to high levels of stimulatory signal (see U.S. Pat. No. 9,528,088). As another example, population of cells may be exposed to polymer-interaction molecule conjugates that stimulate proliferation of one or more cell type while either having no effect or act to inhibit proliferation of one or more other cell type.

CD1 is a family of glycoproteins present on the surfaces of a number of human immune cells including antigen-presenting cells (APCs). These receptors are involved in the presentation of self and non-self lipids (e.g., glycolipids) to natural killer T-cells (NKT cells) as well as other T cells. Presentation of lipids by APCs to T cells often results in T cell proliferation.

In some instances, interaction action molecules may have binding activity for one or more CD1 (e.g., CD1a, CD1b, CD1c, CD1d, and/or CD1e) receptors. Such interaction action molecules may stimulate of inhibit stimulation of such receptors. Interaction action molecules with binding activity for one or more CD1 receptor may be proteins, peptides, lipids, etc.

Also, provide here are compositions and methods for inducing proliferation of T cells (e.g., NKT cells) using polymer-interaction action molecules with binding activity for one or more CD1 receptor (e.g., CD1d receptors). Further, such methods may include the use of APCs or other cells (e.g., a cell engineered to express CD1d receptors) (Kunjo et al., “Invariant NKT cells recognize glycolipids from pathogenic Gram positive bacteria”, Nat. Immunol., 12:966-974 (2012)).

CD117 (also referred to as tyrosine-protein kinase KIT or KIT) receptors are expressed on the surfaces of a number of cells, including hematopoietic stem cells. CD117 receptors are cytokine receptors that stem cell factor (SCF). Binding of CD117 receptors to stem cell factor (SCF) is believed to result in receptor dimerization resulting the activation of intracellular signaling mediated by tyrosine kinase activity.

In some instances, interaction action molecules may have binding activity for CD117 receptors. Such interaction action molecules may stimulate of inhibit stimulation of such receptors. Also, provide here are compositions and methods for inducing proliferation, differentiation, enhanced cell survival, decreased cell survival, etc.

Antigen-presenting cells (APCs) are a group of immune cells of the immune system cells involved in cellular immune response by processing and presenting antigens for lymphocyte (e.g., T cell) recognition. APCs include dendritic cells, macrophages, Langerhans cells and B cells. Dendritic cells are antigen-presenting cells (APC), which may be isolated or generated from human blood mononuclear cells.

Polymer-interaction molecule conjugates include those that may be used to stimulate dendritic cell (DC) expansion and the formation of DCs from other cell types. These include polymer-interaction molecule conjugates comprising granulocyte-macrophage colony-stimulating factor (GM-CSF), tumour necrosis factor-α (TNF-α), interleukin-1β, interleukin-4, interleukin-6, and/or other interleukins (IL), as well as methods of using such polymer-interaction molecule conjugates for the expansion of DCs.

DCs may be prepared by contacting macrophages and/or monocytes with GM-CSF and IL-4. (See Nair et al., Current Protocols in Immunology 99:3.19.1-A.3G.5 (2012).) Further, DCs may be loaded with antigens (e.g., tumour antigens) prior to infusion into subjects.

In some instance, the polymer-interaction molecule conjugates provided herein may be contacted with various types of cells. Table 4 sets out a series of examples of cytokines that may be contacted with types of cells. In many instances, the cytokines set out in Table 4 will used in combination with other interaction molecules (e.g., one or more agonistic receptor binding antibody, one or more additional cytokine, etc.). Thus, provided herein are compositions and methods for activating and/or stimulating the proliferation of one of more cell type, where the methods involve contact the one of more cell type with one or more cytokine set out in Table 4 and, in some instances, one or more additional interaction molecule. Further, one or more of interaction molecules used may be conjugate to a polymer.

TABLE 4

| Cytokine | Cell Type(s) | Cytokine | Cell Type(s) |
|---|---|---|---|
| IL-2 | T cells, NK cells | IL-21 | CD8⁺ T cells, NK T cells, |
| | | | B cells, NK cells |
| IL-7 | T cell subsets | IL-6 | T cells, B cells |
| IL-15 | T cells, NK cells | IL-23 | CD4⁺ T cells, memory |
| | | | T cells |
| IL-4 | Th2 cells, B cells | IL-5 | B cells |
| IL-12 | Th1 cells, NK cells | IL-27 | T cells, B cells, |
| IL-18 | NK cells | | macrophages, dendritic |
| | | | cells, NK cells |

Provided herein are polymer-interaction molecule conjugates that are capable of inducing the activation, differentiation and/or expansion of immune cells (e.g., human immune cells, such as T cells, dendritic cells, macrophages, Langerhans cells, B cells, etc.). Thus, provided herein are also methods for using such polymer-interaction molecule conjugates inducing the activation, differentiation and/or expansion of immune cells.

### Kits

Also provided herein are kits comprising (i) compositions for the isolation of cells (e.g., T cells, dendritic cells, B cells, etc.) from a subject; (ii) compositions for the ex vivo culture of cells (e.g., T cells, dendritic cells, B cells, etc.), and (iii) polymer-interaction molecule compositions. Kits provided herein may optionally include compositions for the re-activation of cells (e.g., T cells such as Treg cells).

Kits can also include written instructions for use of the particular kit, such as instructions for wash steps, culturing conditions, activation and duration of incubation of isolated cells with compositions provided herein for selective expansion of specific cell subtype populations (e.g., T cell subtype populations).

### Examples

### Example 1: Materials and Polymer Preparation

Materials: α-cyclodextrin (α-CD, Sigma, cat. no. 28705), polyethylene glycol diamine 10 kDa (PEG-diamine 10 kDa, Creative PEGWorks, cat. no. PSB-365 (AA-PEG-AA, MW 10 kDa)), polyethylene glycol diamine 35 kDa (PEG-diamine 35 kDa, Creative PEGWorks, cat. no. PSB-3635 (AA-PEG-AA, MW 35 kDa)), 1-adamantaneacetic acid, 1-ethyl-3-(3-dimethylaminopropyl)carbodiimide hydrochloride (EDC), NHS, dimethylformamide (DMF), succinic anhydride, pyridine, diethyl ether, 8-arm polyethylene glycol-acid 20 kDa (8-arm PEG COOH 20 kDa, 7.5 COOH groups, Creative PEGWorks, cat. no. PSB-832), 8-arm polyethylene glycol-acid 40 kDa (8-arm PEG COOH 40 kDa, 7.5 COOH groups, Creative PEGWorks, cat. no. PSB-834), poly(2-ethyl-2-oxazoline)-stat-poly(C3M-COOH) 20 kDa (POx20k-COOH, 24 COOH groups, Avroxa, cat. no. SR12.0180R215.0020/05.01A, poly(2-ethyl-2-oxazoline)-stat-poly(C3M-COOH) 100 kDa (POx100k-COOH, 100 COOH groups, Avroxa, cat. no. SR12.0900R215.0100/05.01A), dendrimer 5 kDa (G3-COOH, 24 COOH groups, Polymer Factory, cat. no. PFD-G3-TMP-COOH), dendrimer 20 kDa (G5-COOH, 96 COOH groups, Polymer Factory, cat. no. PFD-G5-TMP-COOH), hyperbranched PEG 29 kDa (hyperbranched PEG-COOH, 64 COOH groups, Polymer Factory, cat. no. PFLDHB-G5-PEG10k-COOH), 4-(4,6-Dimethoxy-1,3,5-triazin-2-yl)-4-methylmorpholinium chloride (DMTMM), 1-(2-aminoethyl)maleimide hydrochloride, Dulbecco's phosphate-buffered saline (DPBS), 2-(N-morpholino)ethanesulfonic acid (MES), 5-norbornene-2-methylamine, Tris-(2-Carboxyethyl)phosphine (TCEP), tris(hydroxymethyl)aminomethane (Tris), dimethyl sulfoxide (DMSO), and lithium phenyl-2,4,6-trimethylbenzoylphosphinate (LAP). Polystreptavidin may be obtained by contacting Thermo Fisher Scientific (Polymerized Streptavidin, Part No. NCI04155). Polystreptavidin is also available from other sources (e.g., Eagle Biosciences, cat. no. 10 120). Further, methods for producing polystreptavidin are set out in PCT Publication WO 1989/08259, U.S. Pat. Nos. 6,638,728 and 5,268,306.

Unless otherwise stated, all incubations were performed at room temperature.

Receptor Activation Anti-CD3 VHH antibody (RA CD3 VHH) and Receptor Activation Anti-CD28 VHH antibody (RA CD28 VHH) were generated by Thermo Fisher Scientific and are available upon request by contacting at the following email address: captureselectsupport@thermofisher.com., or by otherwise contacting Thermo Fisher Scientific (e.g., US Phone Number (800) 556-2323).

Methods

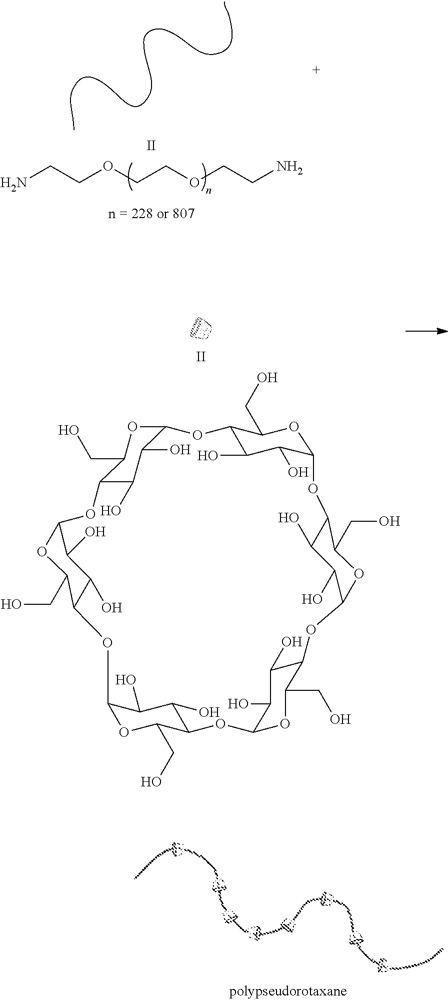

Synthesis of Polypseudorotaxane (PpseudoRX). 0.268 g of α-CD was weighed into a 15 mL glass vial equipped with a stirrer bar. 6 mL of deionized H₂O was added to the vial and α-CD was stirred until complete dissolution. Additional α-CD was added while continuously stirring until a saturated solution of α-CD was achieved. Excess solid α-CD was filtered out with 0.2 μm cellulose ester filters. Following that, 0.1 g of PEG diamines were weighed into a 6 mL glass vial with a stirrer bar. While stirring, the colorless saturated α-CD solution was added to the PEG-diamine and the solutions turned opaque upon addition. Mixtures were left to stir for 18 hours before lyophilized to yield white solids (e.g., PpseudoRX 10 kDa or 35 kDa, depending on the PEG-diamine used), which were used without further purification.

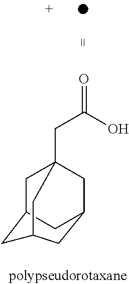 [SMILES: O=C(O)CC12CC3CC(CC(C3)C1)C2]

General Procedure for Capping of PpseudoRX to Produce Polyrotaxane (PRX). In a glass vial equipped with a stir bar, 1-adamantaneacetic acid (0.031 g, 1.60×10⁻⁴ mol), EDC (0.053 g, 2.76×10⁻⁴ mol), and NHS (0.030 g, 2.61×10⁻⁴ mol) was weighed and added. The vial was capped with a rubber septum before addition of 0.5 mL of anhydrous DMF. The mixture was then left to solubilized while purged with argon for 30 minutes. In a separate vial containing PpseudoRX 35 k (0.05 g, 1.43×10⁻⁶ mol) and a stir bar, a rubber septum was also used to cap the vial before purging the solids with argon for 30 minutes. Following that, the deoxygenated mixture was added via syringe to the vial containing PpseudoRX. The reaction mixture was then left to stir for 20 hours. The reaction mixture was then added to approximately 5 mL diethyl ether, where white precipitate was formed and collected via centrifugation. The yielded white solids remained insoluble (PRX) when H₂O was added, which was an indication that the capping was successful, and α-CD remained threaded on the PEG chains.

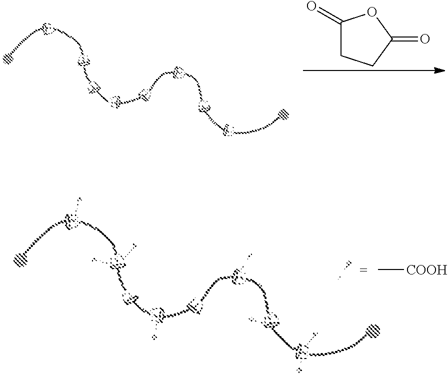

General Procedure to Functionalize PRX with COOH Groups (PRX-COOH). 0.48 g of PRX was weighed into a round bottom flask equipped with a stir bar, followed by addition of 0.516 g of succinic anhydride. The flask was capped with a rubber septum and purged with argon for 20 minutes. 10 mL of pyridine was then added via syringe. The reaction was left to stir for 20 hours. The reaction mixture was added dropwise to approximately 50 mL of diethyl ether. A white precipitate was formed and collected via centrifugation. The white precipitate was further washed twice with diethyl ether. Following that, the white solid was dissolved in deionized H₂O and transferred to a dialysis tubing of 12-14 k Da molecular weight cut off (MWCO). Dialysis was carried out against deionized H₂O for two days with the dialysate changed twice a day. White solid (PRX-COOH) was yielded after the dialyzed solution was lyophilized.

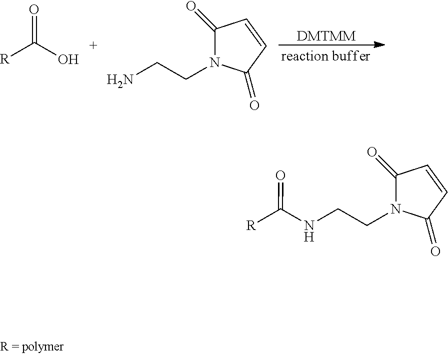

General Procedure for Synthesis of Maleimide-Functionalized Polymers. COOH-functionalized polymer, 1-(2-aminoethyl)maleimide hydrochloride (4-5 equiv. to COOH groups), and DMTMM (2-3 equiv. to COOH groups) were weighed into a vial. A reaction buffer (DPBS pH7-7.3 or MES pH6) was added to the vial and solid reagents were solubilized on a vortex. The reaction mixture was left to react in a thermomixer for 20 hours. The reaction mixture was then transferred to a dialysis tubing of 3.5 k Da MWCO and dialyzed against deionized H₂O at 4° C. A white solid was yielded after lyophilization of dialyzed solution. The degree of maleimide functionalization was determined via nuclear magnetic resonance.

Reduction of Disulfide Bonds on VHH Antibodies. A 50 mM stock solution of TCEP was prepared in 100 mM Tris pH7. The VHH antibody was then mixed with required volumes of the TCEP stock solution (10 equiv.) in 100 mM Tris pH 7, where final concentration of VHH antibody was 5 mg/ml. The reaction was carried out in a thermomixer for 30 minutes at 25° C. The resulting mixture was used without further purification.

Conjugation of VHH Antibodies to Maleimide-Functionalized Polymers. Stock solution of polymers were prepared in 100 mM Tris pH 7 (except for PRX 35 k that was prepared in DMSO and(G5 dendrimers that were prepared either in DMSO or in the above reaction buffer). Stock polymer solutions were then mixed with reduced VHH antibody solutions according to desired VHH antibody-to-polymer ratio. Additional 100 mM Tris buffer pH7 was added to achieve targeted reaction concentrations. The reactions were left to mix in a thermomixer for 20 hours at 25° C. Success of conjugation reactions were verified via size exclusion chromatography (SEC) and gel electrophoresis.

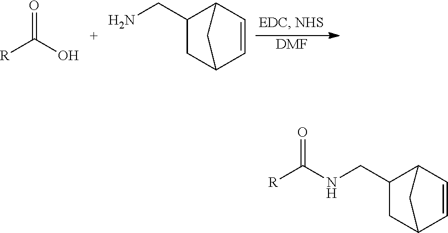

General Procedure for Synthesis of Norbornene-Functionalized Polymers. COOH-functionalized polymer, 5-norbornene-2-methylamine (2.5-3 equiv. to COOH groups), EDC (4.5-5 equiv. to COOH groups), and NHS (4.5-5 equiv. to COOH groups) were weighed into a vial. Anhydrous DMF was added to the vial and solid reagents were solubilized on a vortex. The vial was left to mix on a roller for 20 hours. The reaction mixture was then transferred to a dialysis tubing of 3.5 k Da MWCO and dialyzed against deionized H₂O. A white solid was yielded after lyophilization of dialyzed solution. Degree of maleimide functionalization was determined via nuclear magnetic resonance.

Conjugation of VHH Antibodies to Norbornene-Functionalized Polymers. Stock solutions of polymers and LAP were prepared in DPBS. Stock polymer solutions were mixed with VHH antibodies according to desired VHH antibody-to-polymer ratio and LAP was added to achieve a final concentration of 2.2 mM. Additional DPBS was added to achieve targeted reaction concentrations. The reaction mixtures were then left to irradiate under a 365 nm lamp for 30 min. Success of conjugation reactions were verified via size exclusion chromatography (SEC) and gel electrophoresis.

### Example 2: T Cell Activation

Materials: Peripheral blood mononuclear cells (PBMC, 63.2% CD3, 11.8% CD8, 53.4% CD4, 98.9% viable), S117: inactivated FBS, DYNABEADS™ UNTOUCHED™ Human T Cells Kit (Thermo Fisher Scientific, cat. no. 11344D), Dulbecco's phosphate-buffered saline (DPBS)+0.1% human serum albumin, 2 mM EDTA (Buffer 1), CTS™ OPTMIZER™ (Thermo Fisher Scientific, cat. no. A10221-01) (with 2.5% SR, gentamicin, 100 U IL-2/mL), CTS™ Immune Cell SR (Thermo Fisher Scientific, cat. no. A2596101), Phycoerythrin conjugated anti-CD25 antibody (Thermo Fisher Scientific, cat. no. 12-0259-42), phosphate buffered saline (PBS) (with 0.1% BSA), RA CD3 VHH/biotin. As indicated above, these Camelid-derived, single-domain (VHH) antibody fragments with binding specificity for human CD3 receptors and human CD28 receptors are available by request to Thermo Fisher Scientific at captureselectsupport@thermofisher.com.

Methods and Results

Isolation of T Cells from PBMCs. A vial of cells was thawed in a 37° C. water bath before the cells were transferred to a 50 mL tube. 10 mL of prewarmed 0.1% HSA, 2 mM EDTA was added dropwise to the cells up to 50 mL. Cells were then counted by analysing 20 μL of cell suspension diluted in 10 mL of isotone solution. Cells were centrifuged at 400 g for 8 minutes before the supernatant was discarded. The resulting cell pellet was resuspended to a final concentration of 1×10⁸ cell per mL in Buffer 1. 0.5 mL (5.00×10⁷ cells) resuspended cells was transferred to a 15 mL falcon tube. Following that, T cells were isolated according to the isolation procedure provided by the DYNABEADS™ UNTOUCHED™ Human T Cells Kit. In general, 0.1 mL of heat inactivated fetal bovine serum and fetal calf serum (FBS/FCS) was added to the cells, followed by 0.1 mL of Antibody Mix from the DYNABEADS™ UNTOUCHED™ Human T Cells Kit. The resulting cell suspension was mixed well and incubated for 20 minutes (2 to 8° C.). Cells were then washed with 4 mL of Buffer 1 by tilting the tube several times before centrifugation at 350×g for 8 min (2 to 8° C.). The supernatant was discarded and the cells were resuspended in 0.5 mL of Buffer 1. 0.5 mL of pre-washed DYNABEADS® were added and cell/bead suspension was incubated for 15 minutes (18 to 25° C.) with gentle tilting and rotation. Following that, 4 mL of Buffer 1 was added, and cell/bead suspension was thoroughly mixed with a narrow-tipped pipette (>10 times) while avoiding foaming. The tube was placed on a magnet for 2 minutes and the supernatant containing T cells was transferred to a new tube. This step was repeated by the addition of another 4 mL of Buffer 1 to the DYNABEADS®. The two supernatants were then combined in one 50 mL tube and a cell count was performed by analyzing 20 μL of cell sample diluted in 10 mL Isoton II Diluent (Beckman Coulter, cat. no. 8546719). The cells were then centrifuged for 8 minutes at 350×g before discarding supernatant and resuspension of the cell pellet in CTS™ OPTMIZER™ (with 2.5% SR, gentamicin, 100 U IL-2/mL) (1×10⁶ cells per mL).

Activation of T Cells. A range of solutions with varying concentrations of the different polymer-CD3 reagents (between 0.5 to 16.1 μg/mL) and CD3 DYNABEADS® in PBS were prepared and added to a 96 well plate. 100 μL of CD3 cells (0.1×10⁶ T cells) diluted in CTS™ OPTMIZER™ containing 2.5% CTS™ Immune Cell SR were added to each of the solutions. Cells were then placed in a 37° C. (5% CO₂) incubator for three days.

Cell Staining for Flow Cytometry. Cells were centrifuged, the supernatant discarded, the cell pellets were resuspended in 100 μL Buffer 1 and transferred to wells of a v-bottom 96-well plate (Corning, cat no. 3894). Cells were centrifuged for 7 minutes at 400×g, the supernatants were discarded and cells pellets were resuspended in 50 μL Buffer 1 containing anti-CD25 antibody conjugated to PE at 1:20 dilution. The plate was then incubated for 20 minutes at room temperature. 100 μL Buffer 1 was added to each well and the plate was centrifuged for 7 minutes at 400×g. The supernatants were then discarded, cells were resuspended in 100 μL of PBS 0.1% BSA and analysed on the BD LSRFORTESSA™ X-20 Cell Analyzer using a High Throughput Sampler (HTS).

Expansion and Viability of T Cells. CD3 cells were isolated as described above. 100 ng of anti-CD3 VHH and/or 100 ng anti-CD28 VHH polymer reagent(s) was added to each well in a 48-well plate. 0.1×10⁶ T cells diluted in 450 μL CTS™ OPTMIZER™ (with 2.5% SR, gentamicin, 100 U-IL2/mL) was added to each of the solutions. Cells were placed in a 37° C. (5% CO₂) incubator for eight days. Cells was diluted and transferred to new wells at day 3, 5 and 6 days prior to harvesting for cell counting and viability measurements on a NUCLEOCOUNTER® NC-3000™.

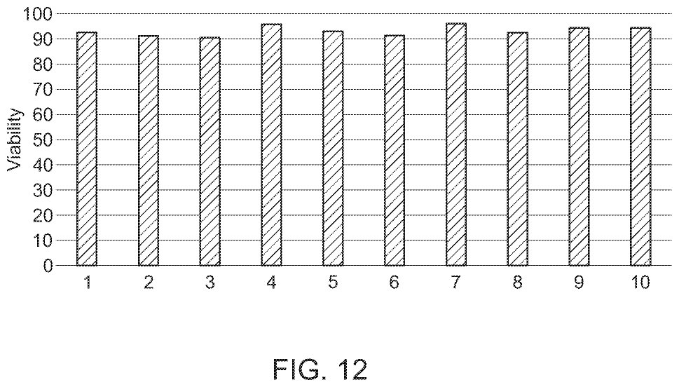

The data presented in FIGS. 12 and 13, and Table 7 were generated using anti-CD3 and anti-CD28 VVH antibodies in soluble form or as a component of a polymer-interaction molecule conjugates.

TABLE 5
CD25 Expression of T Cells Contacted with Anti-CD3 Antibodies

| | | CD25 | CD25 |
|---|---|---|---|
| Sample | μg/mL | Geometric Mean | Percentage |
| RA CD3 VHH | 16.7 | 2606 | 5 |
| | 8.3 | 4767 | 5.77 |
| | 4.2 | 4636 | 5.47 |
| | 2.1 | 3698 | 5.03 |
| | 1.0 | 3141 | 5 |
| | 0.5 | 2689 | 4.71 |
| PRX10k-RA CD3 VHH | 16.7 | 6257 | 59.2 |
| | 8.3 | 5601 | 46.1 |
| | 4.2 | 11494 | 83.7 |
| | 2.1 | 15515 | 93 |
| | 1.0 | 17108 | 96.1 |
| | 0.5 | 19682 | 96.5 |
| POx100k-RA CD3 VHH | 16.7 | 3065 | 12.8 |
| | 8.3 | 4270 | 25.2 |
| | 4.2 | 11114 | 52.5 |
| | 2.1 | 14518 | 79.3 |
| | 1.0 | 15488 | 88.4 |
| | 0.5 | 17302 | 91.9 |
| POx20k-RA CD3 VHH | 16.7 | 2011 | 2.76 |
| | 8.3 | 2754 | 4.81 |
| | 4.2 | 2540 | 4.89 |
| | 2.1 | 3532 | 10.7 |
| | 1.0 | 9382 | 43.2 |
| | 0.5 | 15072 | 72 |
| PRX35k-RA CD3 VHH | 16.7 | 6917 | 59.3 |
| | 8.3 | 14103 | 79.4 |
| | 4.2 | 15021 | 84.6 |
| | 2.1 | 15321 | 86.5 |
| | 1.0 | 16708 | 90.5 |
| | 0.5 | 14747 | 87.7 |
| PEG8arm40k-RA CD3 VHH | 16.7 | 3615 | 65.2 |
| | 8.3 | 11588 | 94 |
| | 4.2 | 14448 | 95.9 |
| | 2.1 | 15660 | 96.2 |
| | 1.0 | 17025 | 96.5 |
| | 0.5 | 13198 | 95.2 |
| Hyperbranched PEG-RA | 16.7 | 3132 | 15.1 |
| CD3 VHH | 8.3 | 3275 | 21.9 |
| | 4.2 | 4846 | 35.4 |
| | 2.1 | 9485 | 52.5 |
| | 1.0 | 11394 | 60.8 |
| | 0.5 | 11567 | 70.6 |
| G3-RA CD3 VHH | 4.2 | 3263 | 14.2 |
| | 2.1 | 3850 | 32.6 |
| | 1 | 4525 | 39.3 |
| | 0.5 | 4609 | 36.8 |
| G5-RA CD3 VHH | 16.7 | 3514 | 13.7 |
| | 8.3 | 6615 | 28.8 |
| | 4.2 | 13807 | 73 |
| | 2.1 | 19771 | 92.8 |
| | 1.0 | 19444 | 95.4 |
| | 0.5 | 16922 | 96.6 |
| PEG8arm20k-RA CD3 VHH | 16.7 | 3625 | 49.5 |
| | 8.3 | 11620 | 92.9 |
| | 4.2 | 18633 | 97 |
| | 2.1 | 18120 | 96.4 |
| | 1.0 | 20859 | 97.3 |
| | 0.5 | 15159 | 96.9 |
| PEG8arm40k-RA CD3 VHH | 8.3 | 9150 | 83.2 |
| (photo) | 4.2 | 15426 | 93.4 |
| | 2.1 | 14138 | 93.3 |
| | 1 | 14252 | 94.4 |
| | 0.5 | 11864 | 93.5 |
| POx20k-RA CD3 VHH | 16.7 | 3273 | 35.5 |
| (photo) | 8.3 | 20911 | 93.1 |
| | 4.2 | 24551 | 97.5 |
| | 2.1 | 22112 | 97.3 |
| | 1.0 | 22312 | 97.9 |
| | 0.5 | 15413 | 97.3 |
| Hyperbranched PEG-RA | 16.7 | 5085 | 15.6 |
| CD3 VHH | 8.3 | 4704 | 24.4 |
| (photo) | 4.2 | 4382 | 33.7 |
| | 2.1 | 4171 | 48 |
| | 1.0 | 3424 | 56.6 |
| | 0.5 | 3108 | 49.6 |
| Polystreptavidin-RA CD3 | 16.7 | 7366 | 40.3 |
| VHH | 8.3 | 7799 | 35.1 |
| | 4.2 | 7736 | 34.5 |
| | 2.1 | 12194 | 69.3 |
| | 1.0 | 12515 | 79.1 |
| | 0.5 | 13002 | 92.3 |
| Non-Stimulated | 0 | 1893 | 1.21 |
| | 0 | 1529 | 0.92 |
| | 0 | 1731 | 1.11 |
| Non-Stimulated Mean | 0 | 1718 | 1.1 |

TABLE 6
CD25 Expression of T Cells Contacted with Anti-CD3 Antibodies

| | | Lymphocytes/ | Lymphocytes/ |
|---|---|---|---|
| | | Single Cells/ | Single Cells/ |
| | | CD25 \| | CD25 \| |
| | | Freq. of | Geometric Mean |
| Sample | μg/mL | Parent | (PE-A) |
| RA CD3 VHH | 16.7 | 9.75 | 3427 |
| | 8.3 | 9.64 | 2629 |
| | 4.2 | 8.8 | 2509 |
| | 2.1 | 9.22 | 2111 |
| | 1.0 | 7.55 | 2025 |
| | 0.5 | 8.17 | 2470 |
| | 0.3 | 6.67 | 1817 |
| | 0.1 | 6.17 | 1522 |
| | 0.07 | 5.63 | 1324 |
| PRX10k-RA CD3 VHH | 16.7 | 17.2 | 2444 |
| | 8.3 | 21.3 | 2725 |
| | 4.2 | 25.9 | 2895 |
| | 2.1 | 49.6 | 5868 |
| | 1.0 | 72 | 12354 |
| | 0.5 | 83 | 10129 |
| | 0.3 | 82.1 | 10638 |
| | 0.1 | 77 | 5964 |
| | 0.07 | 62.8 | 3650 |
| POx100k-RA CD3 VHH | 16.7 | 10.4 | 1589 |
| | 8.3 | 12.9 | 1578 |
| | 4.2 | 24.8 | 3006 |
| | 2.1 | 43.1 | 5309 |
| | 1.0 | 58.7 | 8107 |
| | 0.5 | 70.6 | 10551 |
| | 0.3 | 79.8 | 15539 |
| | 0.1 | 82.8 | 16704 |
| | 0.07 | 89.5 | 22893 |
| POx20k-RA CD3 VHH | 16.7 | 7.12 | 1345 |
| | 8.3 | 8.7 | 1369 |
| | 4.2 | 10.4 | 1729 |
| | 2.1 | 12.4 | 2556 |
| | 1.0 | 17.2 | 3233 |
| | 0.5 | 34.9 | 6063 |
| | 0.3 | 57.5 | 11411 |
| | 0.1 | 69.2 | 11469 |
| | 0.07 | 83.5 | 15493 |
| PRX35k-RA CD3 VHH | 16.7 | 27 | 2309 |
| | 8.3 | 24 | 2310 |
| | 4.2 | 24.6 | 2110 |
| | 2.1 | 25.3 | 2144 |
| | 1.0 | 24.7 | 3000 |
| | 0.5 | 22.2 | 2375 |
| | 0.3 | 25.2 | 2484 |
| | 0.1 | 24.3 | 2622 |
| | 0.07 | 25.7 | 2370 |
| PEG8arm40k-RA CD3 | 16.7 | 40.6 | 3411 |
| VHH | 8.3 | 65.4 | 5689 |
| | 4.2 | 76.4 | 6109 |
| | 2.1 | 57.2 | 4233 |
| | 1.0 | 63.6 | 5718 |
| | 0.5 | 49.4 | 3660 |
| | 0.3 | 33 | 3304 |
| | 0.1 | 19.4 | 3425 |
| | 0.07 | 12 | 2943 |
| Hyperbranched PEG- | 16.7 | 13.9 | 1792 |
| RA CD3 VHH | 8.3 | 16.7 | 1909 |
| | 4.2 | 15.2 | 1680 |
| | 2.1 | 20 | 2098 |
| | 1.0 | 18.9 | 1961 |
| | 0.5 | 19.3 | 1723 |
| | 0.3 | 18.3 | 1504 |
| | 0.1 | 21.5 | 1528 |
| | 0.07 | 25.2 | 1588 |
| G3-RA CD3 VHH | — | — | — |
| | — | — | — |
| | 4.2 | 18.4 | 2864 |
| | 2.1 | 20.7 | 3158 |
| | 1.0 | 21.3 | 2978 |
| | 0.5 | 15.9 | 2555 |
| | 0.3 | 15.1 | 1961 |
| | 0.1 | 10.6 | 1947 |
| | 0.07 | 8.22 | 1417 |
| XR CD3 (monoclonal) | | 72.3 | 13640 |
| Streptavidin | | | |

TABLE 7
Fold expansion and viability on day 8 (200 ng/ml)

| | Sample | Total | Viable Cell | Fold | Viability |
|---|---|---|---|---|---|
| Sample | No. | Cells | Count | Expansion | (%) |
| RA CD28 VHH/RA CD3 VHH | 1 | 450000 | 189,500 | 1 | 92.6 |
| RA CD28 VHH | 2 | 450000 | 126,500 | 1 | 91.2 |
| RA CD3 VHH | 3 | 450000 | 375,500 | 3 | 90.6 |
| PRX10k-RA CD28 VHH/−RA CD3 VHH | 4 | 450000 | 4,070,000 | 54 | 95.85 |
| POx20k-RA CD28 VHH/−RA CD3 VHH | 5 | 450000 | 4,610,000 | 61 | 93.05 |
| POx100k-RA CD28 VHH/−RA CD3 | 6 | 450000 | 4,100,000 | 55 | 91.3 |
| VHH | | | | | |
| PEG8arm20k-RA CD28 VHH/−RA CD3 | 7 | 450000 | 3,460,000 | 46 | 96.1 |
| VHH | | | | | |
| POx20k-RA CD28 VHH/RA CD3 VHH | 8 | 450000 | 3,620,000 | 48 | 92.45 |
| (Co-Conjugated) | | | | | |
| G5-RA CD28 VHH/−RA CD3 VHH | 9 | 450000 | 3,370,000 | 45 | 94.35 |
| RA CD28 VHH, POx20k-RA CD3 VHH | 10 | 450000 | 4,010,000 | 53 | 94.35 |

### Example 3: Activation and Expansion of T Cells Dendrimers (Part 1)

Methods: 5 million isolated human T cells were seeded in 5 mL complete CTS OpTmizer medium (basal medium with 2.5% CTS immune cell serum replacement (1 million cells/mL), 2.6% CTS OPTMIZER™ subunit, 1% L-Glutamine and 0.13% Gentamicin).

Maleimide derivatized G5 dendrimers were conjugated with anti-CD3 VHH (G5-CD3) and anti-CD28 VHH (G5-CD28) separately and in combination. Two lots of dendrimers were tested as separate conjugates, referred to herein as Lot 1 and Lot 2. The concentration of conjugates was 50 ng/mL for each. For mixed prototypes, G5-CD3 AFP Lot 1 plus G5-CD28 AFP Lot 1 and G5-CD3 AFP Lot 2 plus G5-CD28 AFP Lot 2 were used at a total of 100 ng/mL. G5 dendrimers conjugates with both anti-CD3 VHH and anti-CD28 VHH (G5-CD3-CD28) were used at a concentration of 50 ng/mL.

CTS DYNABEADS™ CD3/CD28 (Thermo Fisher Scientific, cat. no. 40203D) were added so that a final bead to cell ratio (B:C) of 1:1 was achieved. Cell activation (CD69 and CD25 expression) was measured via flow cytometry on Day 1 and Day 3. On Day 3, the cells were washed and beads were removed from CTS control (removing of activating reagents) and placed in fresh medium CTS OpTmizer medium. On day 6, cells were stained and analyzed by flow cytometry to assess fold expansion.

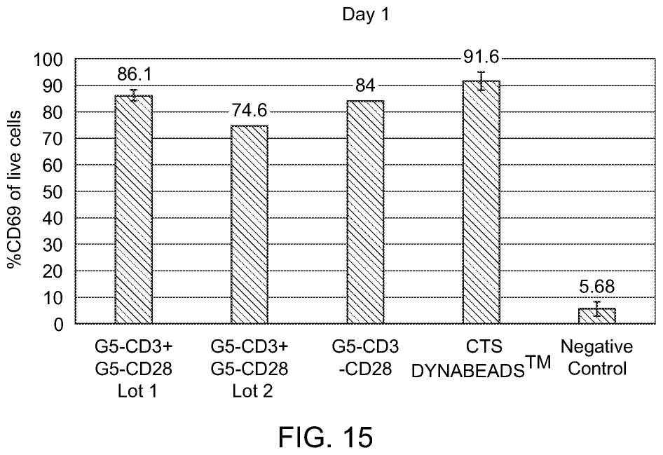

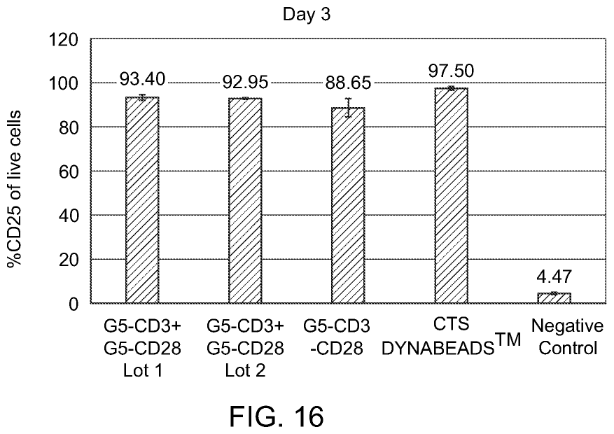

Results: As shown in FIGS. 15-16 and Tables 8-9, the contacting of T cells with G5 dendrimers conjugated to receptor stimulatory anti-CD3 and anti-CD28 VHHs result in a substantial increase in the percentage of cells expressing CD69 and CD25 (IL2 receptor alpha chain) on, respectively, days 1 and 3 of culture. Further, the above effects are seen when anti-CD3 and anti-CD28 VHHs are conjugated to dendrimers together or separately.

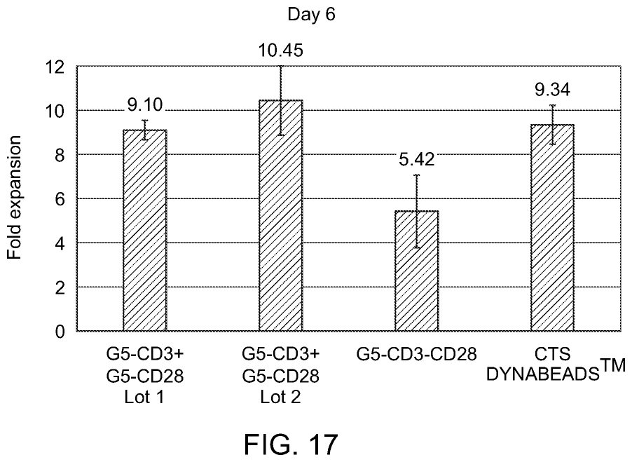

FIG. 17 and Table 10 show data related to the fold expansion of T cells on day 6. As can be seen, anti-CD3 and anti-CD28 VHHs are conjugated to G5 dendrimers are capable of inducing the expansion of T cells.

TABLE 8
(shown in FIG. 15) Day 1

| | MFI CD69 | CD69+ Freq. of |
|---|---|---|
| | (Geometric Mean) | Parent (%) |
| | Average | |
| G5-CD3+ G5-CD28 | 5832 | 86.1 |
| Lot 1 | | |
| G5-CD3+ G5-CD28 | 3172 | 74.6 |
| Lot 2 | | |
| G5-CD3 − G5-CD28 | 6248 | 84 |
| CTS | 12493 | 91.6 |
| Negative control | 174 | 5.68 |
| | Standard Deviation | |
| G5-CD3+ G5-CD28 | 960.25 | 2.12 |
| Lot 1 | | |
| G5-CD3+ G5-CD28 | | |
| Lot 2 | | |
| G5-CD3 − G5-CD28 | | |
| CTS DYNABEADS ™ | 8482.45 | 3.394 |
| Negative control | 60.811 | 2.67 |

TABLE 9
(shown in FIG. 16) Day 3

| | MFI CD25 | CD25+ |
|---|---|---|
| | (Geometric Mean) | Freq. of Parent (%) |
| | Average | |
| G5-CD3+ G5-CD28 | 12701.5 | 93.4 |
| Lot 1 | | |
| G5-CD3+ G5-CD28 | 8793.5 | 92.95 |
| Lot 2 | | |
| G5-CD3 − G5-CD28 | 7273.5 | 88.65 |
| CTS DYNABEADS ™ | 16517.5 | 97.5 |
| Negative Control | 265 | 4.47 |
| | Standard deviation | |
| G5-CD3+ G5-CD28 | 1238.14 | 1.27 |
| Lot 1 | | |
| G5-CD3+ G5-CD28 | 64.35 | 0.35 |
| Lot 2 | | |
| G5-CD3 − G5-CD28 | 1614.32 | 4.17 |
| CTS DYNABEADS ™ | 1098.14 | 0.85 |
| Negative Control | 14.14 | 0.49 |

TABLE 10
(shown in FIG. 17) Day 6, Fold Expansion

| | Average | SD |
|---|---|---|
| G5-CD3+ G5-CD28 | 9.10 | 0.43 |
| Lot 1 | | |
| G5-CD3+ G5-CD28 | 10.45 | 1.57 |
| Lot 2 | | |
| G5-CD3 − G5-CD28 | 5.42 | 1.65 |
| CTS DYNABEADS ™ | 9.34 | 0.88 |

### Example 4: Activation and Expansion of T Cells Dendrimers (Part 2)

Methods: Isolated human T cells were labeled with CFSE dye (Thermo Fisher Scientific, CELLTRACE™ CFSE Cell Proliferation Kit, cat. no. C34554), according to the manufacturer's instructions, and then seeded in complete CTS OPTMIZER™ medium in 96 well U bottom plates. The T cells were then activated with either CTS DYNABEADS™ CD3/CD28 (Thermo Fisher Scientific, cat. no. 40203D) or G5 dendrimer conjugates (G5-CD3 AFP Lot 1 plus G5-CD28 AFP Lot 1) at a cell concentration of 250,000 cells/mL. The T cells were split when needed, but activation reagents were not washed off. T cell activation and expansion was measured by flow cytometric analyses after 4 hours and on Day 1, Day 2, Day 3 and Day 6.

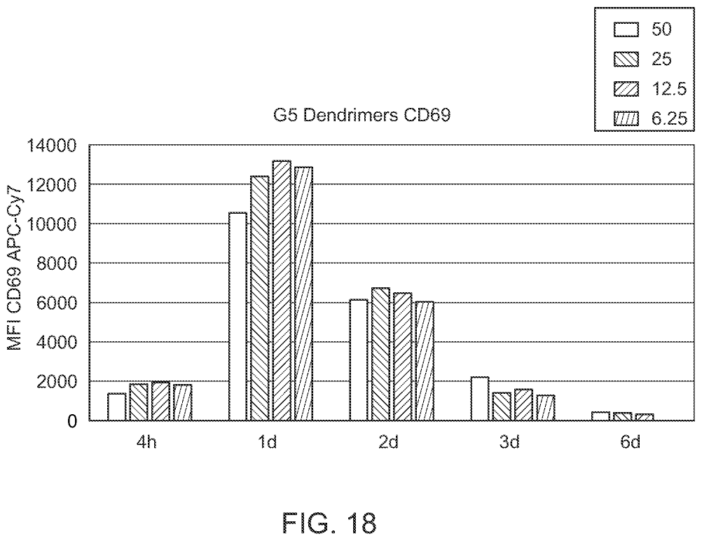

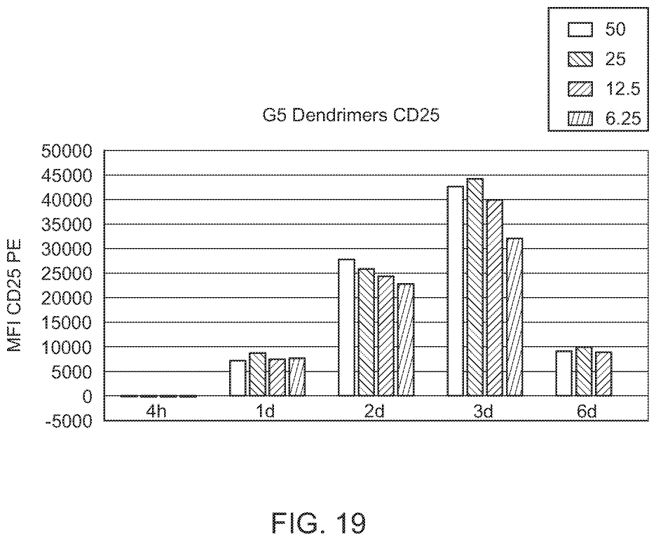

Results: As shown in FIGS. 18 and 19 and Table 11, the contacting of T cells with different concentrations of G5 dendrimers conjugated to receptor stimulatory anti-CD3 and anti-CD28 VHHs results in a substantial increase in the percentage of cells expressing CD69 and CD25 (IL2 receptor) receptors over a six day time period.

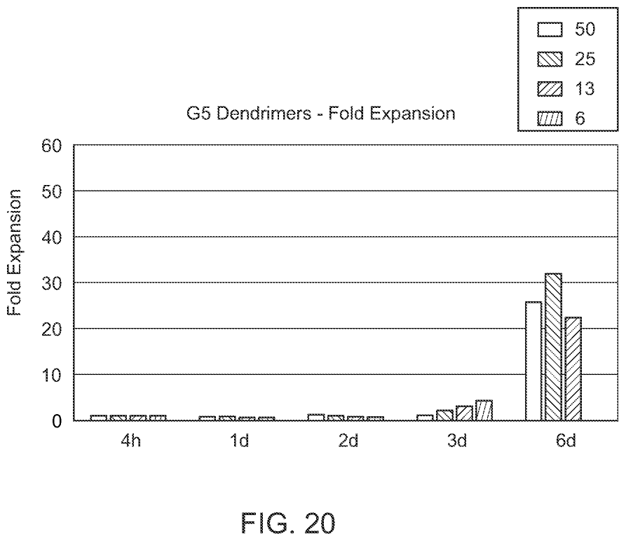

FIG. 20 and Table 13 show data related to the fold expansion of T cells over a 6 day time. As can be seen, from the data, when T cells are contacted with 13 to 50 ng/mL of G5-CD3 AFP and G5-CD28 AFP dendrimers, T cell expansions of 23.9 to 32 fold was seen after 6 days.

TABLE 11
(shown in FIG. 19): G5-Dendrimer, Induced CD69 and CD25
Receptor Expression Mean Fluorescence Intensity (MFI)

| ng/ml | 4 h | 1 d | 2 d | 3 d | 6 d | |
|---|---|---|---|---|---|---|
| 50 | 3978 | 3512 | 3297 | 2759 | 938 | AF647 |
| 25 | 3293 | 3106 | 2458 | 2060 | 560 | |
| 12.5 | 2221 | 2492 | 1796 | 1305 | 309 | |
| 6.25 | 1329 | 1869 | 1252 | 851 | | |
| 50 | 1370 | 10546 | 6144 | 2207 | 421 | CD69 |
| 25 | 1846 | 12404 | 6713 | 1401 | 382 | |
| 12.5 | 1935 | 13182 | 6463 | 1580 | 307 | |
| 6.25 | 1802 | 12858 | 6019 | 1273 | | |
| 50 | 8230 | 2068 | 1310 | 491 | 98 | CFSE |
| 25 | 9367 | 2020 | 1267 | 443 | 110 | |
| 12.5 | 9491 | 2063 | 1254 | 445 | 107 | |
| 6.25 | 9807 | 2056 | 1224 | 436 | | |
| 50 | −64.4 | 7156 | 27799 | 42651 | 9111 | CD25 |
| 25 | −154 | 8748 | 25849 | 44201 | 9899 | |
| 12.5 | −160 | 7474 | 24393 | 39944 | 8902 | |
| 6.25 | −196 | 7688 | 22781 | 32077 | | |

TABLE 12
CTS DYNABEADS ™ CD3/CD28 Bead Induced CD69 and CD25
Receptor Expression Mean Fluorescence Intensity (MFI)

| B:C | 4 h | 1 d | 2 d | 3 d | 6 d | |
|---|---|---|---|---|---|---|
| 1:1 | −3.62 | 324 | 217 | 146 | 47.2 | AF647 |
| 1:2 | −7.48 | 393 | 177 | 110 | 27.9 | |
| 1:4 | −11.2 | 252 | 158 | 44.5 | 21.1 | |
| 1:8 | −12.9 | 189 | 94.1 | 43.1 | 19.5 | |
| 1:1 | 1228 | 17021 | 12060 | 4602 | 1041 | CD69 |
| 1:2 | 1066 | 16713 | 12023 | 2794 | 483 | |
| 1:4 | 603 | 13130 | 10113 | 1458 | 302 | |
| 1:8 | 216 | 8919 | 5732 | 1094 | 218 | |
| 1:1 | 8581 | 2075 | 1371 | 563 | 170 | CFSE |
| 1:2 | 9040 | 2117 | 1405 | 515 | 146 | |
| 1:4 | 8243 | 2038 | 1481 | 511 | 121 | |
| 1:8 | 7765 | 2033 | 1444 | 526 | 98.2 | |
| 1:1 | −63.6 | 7403 | 24875 | 35499 | 25041 | CD25 |
| 1:2 | −76.4 | 6794 | 22510 | 40720 | 16811 | |
| 1:4 | −91.3 | 5569 | 21084 | 42930 | 7899 | |
| 1:8 | −54.5 | 4180 | 16309 | 27902 | 2697 | |

TABLE 13
(shown in FIG. 20): G5-Dendrimer
Induced T Cell Expansion (Fold)

| ng/mL | 4 h | 1 d | 2 d | 3 d | 6 d |
|---|---|---|---|---|---|
| 50 | 1 | 0.84 | 1.25 | 1.08 | 25.79 |
| 25 | 1 | 0.86 | 1.05 | 2.23 | 32.00 |
| 13 | 1 | 0.69 | 0.77 | 3.10 | 22.39 |
| 6 | 1 | 0.65 | 0.75 | 4.29 | 0 |

TABLE 14
CTS DYNABEADS ™ CD3/CD28 Beads
Induced T Cell Expansion (Fold)

| B:C | 4 h | 1 d | 2 d | 3 d | 6 d |
|---|---|---|---|---|---|
| 1:1 | 1 | 2.31 | 3.06 | 5.62 | 55.32 |
| 1:2 | 1 | 2.12 | 3.22 | 9.63 | 55.45 |
| 1:4 | 1 | 1.41 | 2.12 | 7.89 | 53.57 |
| 1:8 | 1 | 1.15 | 0.87 | 3.967 | 40.07 |

Details of one or more embodiments are set forth in the accompanying drawings. Other features, objects, and advantages will be apparent from the description, drawings, and claims. Although a number of embodiments provided herein have been described, it will be understood that various modifications may be made without departing from the spirit and scope of compositions and methods provided herein. It should also be understood that the appended drawings are not necessarily to scale, presenting a somewhat simplified representation of various features and basic principles of compositions and methods provided herein.

All patent documents set out herein are individually incorporated by reference here in their entireties, including U.S. Pat. Nos. 5,268,306, 6,638,728, 8,617,884, 9,040,666, 9,528,088, 10,196,631, US Patent Publication No. 2019/0062706 A1, U.S. Patent Publication No. 2017/0313772 A1, US Patent Publication 2022/0288216 A1, PCT Publication WO 1989/08259, PCT Publication WO 2013/036585, PCT Publication WO 2014/048920, and PCT Publication WO 2017/072251.

1. A polymer-interaction molecule conjugate comprising a dendrimer, wherein the interaction molecule comprises a protein that is capable of binding to the surface of a mammalian cell selected from the group consisting of:
a. a T cell,
b. a natural killer cells,
c. a dendritic cell, and
d. an antigen presenting cell.

2. The polymer-interaction molecule conjugate of claim 1, wherein the interaction molecule is one or more molecule selected from the group consisting of:
a. a variable heavy-heavy (VHH) chain antibody,
b. an anti CD3 antibody,
c. an anti CD4 antibody,
d. an anti CD5 antibody,
e. an anti CD56 antibody,
f. an anti CD8 antibody,
g. an anti CD25 antibody,
h. an anti CD27 antibody,
i. an anti CD28 antibody,
j. an anti CD137 antibody, and
k. an anti CD278 antibody.

3. The polymer-interaction molecule conjugate of claim 2, wherein the interaction molecule is an anti-CD3 VHH antibody.

4. The polymer-interaction molecule conjugate of claim 3, wherein the polymer-interaction molecule conjugate further comprises a cytokine and wherein the cytokine is one or more cytokine selected from the group consisting of: IL-1 beta, IL-2, IL-4, IL-5, IL-6, IL-7, IL-10, IL-12, IL-13, IL-15, IL-18, IL-21, IL-23, IL-27, IFN-gamma, and TGF-beta.

5. The polymer-interaction molecule conjugate of claim 1, wherein the interaction molecule is attached to the polymer through a covalent bond with the linker moiety.

6. (canceled)

7. The polymer-interaction molecule conjugate of claim 5, wherein the linker moiety is maleimide or norbornene.

8. The polymer-interaction molecule conjugate claim 5, wherein one half of the linker moiety is attached to the polymer and the other half of the linker moiety is attached to the interaction molecule.

9.-11. (canceled)

12. A method for activating T cells in a population of T cells, the method comprising contacting the population of T cells with one or more polymer-interaction molecule conjugate comprising an anti CD3 antibody under conditions that allow for the activation of CD3 receptors on T cells in the population,
wherein the polymer is a dendrimer, a polyrotaxane, a polyoxazoline, a polystreptavidin, or a derivative of one of these polymers.

13. The method of claim 12, wherein the polymer is a dendrimer.

14. The method of claim 12, further comprising contacting the population of T cells with one or more polymer-interaction molecule conjugate comprising an anti CD28 antibody under conditions that allow for the activation of CD28 receptors on T cells in the population.

15. The method of claim 14, wherein the anti CD3 antibody and the anti CD28 antibody are conjugated to the same or different polymer molecules.

16.-19. (canceled)

20. The method of claim 12, wherein T cells in the population of T cells are also contacted with one or more cytokine.

21. The method of claim 20, wherein at least one of the one or more cytokine is conjugated to a polymer to form a polymer-interaction molecule conjugate.

22. The method of claim 20, wherein at least one of the one or more cytokine is not conjugated to a polymer.

23.-26. (canceled)

27. The method of claim 12, wherein the polymer has a molecular weight between 0.5 kilodaltons and 150 kilodaltons.

28.-32. (canceled)

33. The method of any one of claim 14, wherein the T cells in the population of T cells are infused into a patient.

34.-35. (canceled)

36. The method of claim 12, wherein the T cells in the population of T cells are separated from the one or more polymer-interaction molecule conjugate.

37.-46. (canceled)

47. A polymer-interaction molecule conjugate comprising an anti CD3 antibody, an anti CD28 antibody, or both an anti CD3 antibody and an anti CD28 antibody, where at least one of the anti CD3 antibody or anti CD28 antibody is a variable heavy-heavy domain (VHH) antibody.

48.-63. (canceled)

64. A method for inducing the activation or proliferation of a mammalian cell, the method comprising contacting the mammalian cell with a first interaction molecule capable of inducing activation or proliferation of the mammalian cell alone or in combination with a second interaction molecule, wherein the first interaction molecule is a variable heavy-heavy chain (VHH) antibody capable of stimulating a cell surface receptor.

65. The method of claim 64, wherein at least one of the first interaction molecule or the second interaction molecule are conjugated to a polymer to form a polymer-interaction molecule.

66.-69. (canceled)
